(19) United States
(12) Patent Application Publication
(10) Pub. No.: US 2024/0254243 A1
(43) Pub. Date: Aug. 1, 2024

(54) SYSTEMS AND METHODS FOR IMPROVED TARGETED THERAPY

(71) Applicant: Prometheus Laboratories Inc., San Diego, CA (US)

(72) Inventor: Thierry DERVIEUX, San Diego, CA (US)

(21) Appl. No.: 18/563,818

(22) PCT Filed: Jun. 16, 2022
(86) PCT No.: PCT/US2022/033757

§ 371 Date: Nov. 22, 2023

Related U.S. Application Data
(60) Provisional application No. 63/211,954, filed on Jun. 17, 2021.
(60) Provisional application No. 63/313,226, filed on Feb. 23, 2022.

(51) Int. Cl. C07K 16/28; C07K 16/24; G01N 33/564; G16H 10/40; G16H 20/10

(52) U.S. Cl. CPC C07K 16/2866; C07K 16/241; G01N 33/564; G16H 10/40; G16H 20/10; C07K 2317/21; C07K 2317/24; G01N 2800/52

(57) ABSTRACT

Provided herein are systems and methods for optimizing a biological therapy regimen for a subject. The subject may be a patient diagnosed with an immune mediated inflammatory disease. In some embodiments, the systems and methods may involve inputting patient data into a model to forecast a drug concentration level in a patient and establish a dosing regimen for maintaining a pre-specified threshold drug concentration level in the patient. The pre-specified threshold may be a target concentration level for effective treatment of the immune mediated inflammatory disease in the patient.

### CROSS-REFERENCE

This application claims the benefit of U.S. Provisional Application No. 63/211,954, filed Jun. 17, 2021, and U.S. Provisional Application No. 63/313,226, filed Feb. 23, 2022, each of which is incorporated herein by reference in its entirety.

### SUMMARY

In some aspects, the present disclosure provides a method for treating an immune mediated inflammatory disease in a subject, the method comprising: (a) analyzing a biological sample obtained from a subject with an immune-mediated inflammatory disease, wherein the analyzing comprises: (i) obtaining or having obtained the biological sample from the subject; and (ii) quantifying or having quantified analytes in the biological sample, wherein the analytes comprise: (1) a level of a biologic drug, (2) a level of autoantibodies against the biologic drug, and (3) a level of albumin, wherein the subject has received a treatment for the immune-mediated inflammatory disease that comprises a current dose of the biologic drug administered to the subject at a current inter-dose interval; (b) determining a likelihood of achieving a pre-specified threshold concentration of the biologic drug in the subject based, at least in part, on (1) the level of the biologic drug, the level of the autoantibodies, and the level of albumin quantified in (a)(ii), and (2) the current dose of the biologic drug and the current inter-dose interval; and (c) if the likelihood of achieving the pre-specified threshold concentration of the biologic drug is above 50%, then: (1) administering the current dose of the biologic drug to the subject at the current inter-dose interval; or (2) administering a dose of the biologic drug that is (i) lower than the current dose to the subject at the current inter-dose interval, (ii) the same as the current dose to the subject at an inter-dose interval that is longer than the current inter-dose interval, or (iii) lower than the current dose to the subject at an inter-dose interval that is longer than the current inter-dose interval; (d) if the likelihood of achieving the pre-specified threshold concentration of the biologic drug is below 50%, then administering a dose of the biologic drug that is (i) higher than the current dose to the subject in the current inter-dose interval, (ii) the current dose at an inter-dose interval that is shorter than the current inter-dose interval, or (iii) higher than the current dose to the subject in the inter-dose interval that is shorter than the current inter-dose interval; or (e) if the dose of the biologic drug in (d) is above a maximum dose and the inter-dose interval in (d) is less than or equal to a minimum inter-dose interval, then discontinuing the treatment comprising the biologic drug.

In some cases, the biologic drug comprises an antibody or antigen-binding fragment thereof. In some cases, the antibody comprises a monoclonal antibody. In some cases, the biologic drug comprises adalimumab (ADA). In some cases, the current dose of the biologic drug is 40 milligrams (mg), and the current inter-dose interval is every two weeks. In some cases, the dose of the biologic drug in (c) is 20 to 80 mg and the inter-dose interval in (c) is every week to every six weeks. In some cases, the maximum dose in (e) is 80 mg, and the minimum inter-dose interval is weekly. In some cases, the biologic drug comprises infliximab (IFX). In some cases, the current dose of the biologic drug is about 5 milligrams per kilogram (mg/kg), and the current inter-dose interval is every eight weeks. In some cases, the dose of the biologic drug in (c) is about 3 to 15 mg/kg and the inter-dose interval in (c) is every four to twelve weeks. In some cases, the maximum dose in (e) is 15 mg/kg, and the minimum inter-dose interval is every four weeks. In some cases, the biologic drug comprises tocilizumab (TCZ). In some cases, the current dose of the biologic drug is 162 milligrams (mg), and the current inter-dose interval is every two weeks. In some cases, the dose of the biologic drug in (c) is 162 mg and the inter-dose interval in (c) is twice a week to every six weeks. In some cases, the maximum dose in (e) is 162 mg, and the minimum inter-dose interval is twice a week. In some cases, the predetermined threshold concentration of the biologic drug comprises between about 1 mg/L and 10 mg/L. In some cases, the predetermined threshold concentration of the biologic drug comprises about 5 mg/L to about 10 mg/L when the biologic drug is ADA. In some cases, the predetermined threshold concentration of the biologic drug comprises about 5 mg/L to about 10 mg/L when the biologic drug is IFX. In some cases, the predetermined threshold concentration of the biologic drug comprises about 1 mg/L to about 7.5 mg/L when the biologic drug is TCZ.

In some cases, the determining the likelihood of achieving the pre-specified threshold concentration of the biologic drug in the subject is further based, at least in part, on a weight of the subject. In some cases, the determining the likelihood of achieving the pre-specified threshold concentration of the biologic drug in the subject comprises estimating a clearance rate of the biologic drug in the subject based, at least in part, on the weight of the subject and the level of albumin quantified in (a)(ii). In some cases, the determining the likelihood of achieving the pre-specified threshold concentration of the biologic drug in the subject further comprises determining whether the subject has a poor prognostic factor of pharmacokinetic origin (PPFPK), wherein the PPFPK is determined based, at least in part, on the level of the biologic drug quantified in (a)(ii) and the clearance rate. In some cases, if the treatment comprising the biologic drug is discontinued in (e), then administering to the subject a small molecule inhibitor of a Janus Kinase (JAK) or a sphingosine 1-phosphate (S1P) receptor modulator. In some cases, (a) the small molecule inhibitor of JAK comprises baricitinib, tofacitinib, or upadacitinib, or any combination thereof; and (b) wherein the SIP receptor modulator comprises fingolimod, siponimod, ozanimod, or ponesimod, or any combination thereof.

In some cases, if the treatment comprising the biologic drug is discontinued in (e), then administering to the subject another biologic drug that differs from the biologic drug. In some cases, the determining the likelihood of achieving the pre-specified threshold concentration of the biologic drug in the subject comprises applying a algorithm to the analytes quantified in (a)(ii). In some cases, the algorithm comprises a Naive Bayes classifier algorithm. In some cases, the algorithm comprises a Metropolis Hastings algorithm. In some cases, the determining the likelihood of achieving the pre-specified threshold concentration of the biologic drug in the subject in (b) comprises utilizing a model comprising: (i) establishing a first set of parameter estimates from a reference population, wherein the reference population has received the biologic drug for treatment of the immune-mediated inflammatory disease; (ii) deriving a second set of parameter estimates for the model based at least in part on the first set of parameter estimates established in (i); and (iii) inputting data comprising (i) the analytes quantified in the biological sample obtained from the subject into the model and (ii) the current dose of the biologic drug and the current inter-dose interval; and (iv) interrogating the model based at least in part based on the data, wherein the subject is not a part of the reference population. In some cases, the data further comprises a level of a level of C-Reactive Protein (CRP). In some cases, the data further comprises a level of interleukin 6 (IL-6). In some cases, the data further comprises a weight of the subject. In some cases, the data further comprises a body mass index (BMI) of the subject. In some cases, the determining the likelihood of achieving the pre-specified threshold concentration of the biologic drug in the subject is further based, at least in part, on a weight of the subject. In some cases, the biological sample comprises a serum sample. In some cases, the likelihood that high is equal to or about 90%. In some cases, the analytes quantified in (a)(ii) further comprise a level of C-Reactive Protein (CRP). In some cases, the analytes quantified in (a)(ii) further comprise interleukin 6 (IL-6). In some cases, the analytes quantified in (a)(ii) are quantified with an assay comprising a mobility shift assay or a solid-phase immunoassay. In some cases, the solid-phase immunoassay comprises an enzyme-linked immunoassay (ELISA). In some cases, further comprising receiving information about the subject, wherein the information comprises a severity of the immune-mediated inflammatory disease or a symptom thereof. In some cases, the severity of the immune-mediated inflammatory disease comprises a disease remission, a disease recurrence, a disease type, or any combination thereof. In some cases, the severity of the symptom of the immune-mediated inflammatory disease comprises a frequency of the symptom, a type of the symptom, or a combination thereof. In some cases, the severity of the immune-mediated inflammatory disease or symptom thereof is based on a clinical disease activity index (CDAI) score/In some cases, the receiving the information about the subject comprises receiving one or more electronic medical records (EMRs), wherein the one or more EMRs comprise the information. In some cases, the information is self-reported by the subject. In some cases, the information is self-reported by the subject inputting the information into a mobile application on a personal electronic device of the subject.

In some cases, the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some cases, the IBD comprises Crohn's disease (CD). In some cases, the IBD comprises ulcerative colitis (UC).

In some cases, the subject has received the treatment comprising the current dose of the biologic drug administered to the subject at the current inter-dose interval for at least 14 contiguous weeks. In some cases, the subject has received the treatment regimen comprising the current dose of the biologic drug administered to the subject at the current inter-dose interval at least once. In some cases, the biological sample is obtained in (a)(i) up to 20 days following a last administration of the biologic drug at the current dose.

In some further aspect, the present disclosure provides a method for achieving a threshold biologic drug concentration value in a subject, the method comprising: (a) initializing a model of a biologic drug concentration profile for a biologic drug, wherein the model comprises data received from a reference population that were or currently are being treated with the biologic drug for treatment of an immune-mediated inflammatory disease; (b) generating subject specific parameters relating to pharmacokinetic performance of the biologic drug in the subject; (c) simulating the biologic drug concentration profile for the subject based on the subject specific parameters and the data from the reference population; (d) updating the model based on newly received data from the subject in (b) and newly received data from the reference population in (a); and (e) estimating a dose of the biologic drug at an inter-dose interval to achieve the threshold biologic drug concentration value in the subject with the model updated in (d), wherein the threshold biologic drug concentration is sufficient to treat the immune-mediated inflammatory disease in the subject.

In some cases, generating the subject specific parameters comprises compiling information about the subject. In some cases, the information about the subject comprises a severity of the immune-mediated inflammatory disease or a symptom thereof. In some cases, the data is received from the subject via a mobile application on a personal electronic device of the subject. In some cases, the severity of the immune-mediated inflammatory disease comprises a disease remission, a disease recurrence, a disease type, or any combination thereof. In some cases, the severity of the symptom of the immune-mediated inflammatory disease comprises a frequency of the symptom, a type of the symptom, or a combination thereof. In some cases, the severity of the immune-mediated inflammatory disease or symptom thereof is based on a clinical disease activity index (CDAI) score. In some cases, the information about the subject comprises a weight or body mass index (BMI) of the subject. In some cases, the data is contained in one or more electronic medical records (EMRs).

In some cases, the subject specific parameters comprise two or more of: (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or j) log transformed covariates on the subject specific parameters in one or more of (a) to (g) as determined using non-linear mixed effect modeling; or (k) any combination thereof. In some cases, the data received from the reference population comprises: (a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5); (b) a weight of individuals in the reference population; (c) a body mass index (BMI) of the individuals in the reference population; or (d) any combination of (a) to (c). In some cases, the newly received data from the subject comprises: (a) a level of one or more analytes in a biological sample obtained from the subject, wherein the one or more analytes comprises (1) a biologic drug, (2) autoantibodies against the biologic drug, (3) albumin, (4) IL-6, (5) CRP, or (6) any combination of (1) to (5); (b) a weight of the subject; (c) BMI of the subject; or (d) any combination of (a) to (c).

In some cases, estimating the dose of the biologic drug at the inter-dose interval to achieve the threshold biologic drug concentration value in the subject comprises estimating a clearance rate of the biologic drug in the subject based, at least in part, on the weight of the subject and the level of albumin in the biological sample obtained from the subject in the newly received data from the subject. In some cases, estimating the dose of the biologic drug at the inter-dose interval to achieve the threshold biologic drug concentration value in the subject further comprises determining whether the subject has a poor prognostic factor of pharmacokinetic origin (PPFPK), wherein the PPFPK is determined based, at least in part, on the level of the biologic drug in (a)(1) and the clearance rate. In some cases, the level of one or more analytes in a biological sample obtained from the subject is measured using a mobility shift assay or a solid-phase immunoassay. In some cases, the solid-phase immunoassay comprises an enzyme-linked immunoassay (ELISA). In some cases, the biological sample comprises a serum sample. In some cases, the biological sample is obtained from the subject up to 20 days following a last administration of the biologic drug to the subject.

In some cases, the model comprises a trained model. In some cases, the model comprises a Bayesian assimilation. In some cases, the model comprises anon-linear mixed effects model (NLME). In some cases, the model comprises a Markov Chain Monte Carlo (MCMC) simulation. In some cases, estimating the dose of the biologic drug at the inter-dose interval is performed with greater than a 50% confidence. In some cases, estimating the dose of the biologic drug at the inter-dose interval is performed with between about 50% and 90% confidence. In some cases, estimating the dose of the biologic drug at the inter-dose interval is performed with greater than or equal to about a 90% confidence.

In some cases, the biologic drug comprises an antibody or antigen-binding fragment thereof. In some cases, the antibody comprises a monoclonal antibody. In some cases, the biologic drug comprises adalimumab (ADA). In some cases, the dose of the ADA is 40 mg and the inter-dose interval is every two weeks. In some cases, the dose of the ADA is less than or equal to about 80 mg and the inter-dose interval is greater than or equal to about every week. In some cases, the biologic drug comprises infliximab (IFX).

In some cases, the dose of the IFX is 5 mg/kg and the inter-dose interval is every eight weeks. In some cases, the dose of the IFX is less than or equal to about 15 mg/kg and the inter-dose interval is greater than or equal to about four weeks. In some cases, the biologic drug comprises tocilizumab (TCZ). In some cases, the dose of the TCZ is 162 mg and the inter-dose interval is every two weeks. In some cases, the dose of the TCZ is less than or equal to about 162 mg and the inter-dose interval is greater than or equal to about twice a week. In some cases, the threshold biologic drug concentration value comprises between about 1 mg/L and 10 mg/L. In some cases, the threshold biologic drug concentration value comprises about 5 to 10 mg/L when the biologic drug is ADA. In some cases, the threshold biologic drug concentration value comprises about 5 to 10 mg/L when the biologic drug is IFX. In some cases, the threshold biologic drug concentration value comprises about 1 to 7.5 mg/L when the biologic drug is TCZ.

In some cases, further comprising providing a recommendation to discontinue treatment of the immune-mediated inflammatory disease with the biologic drug if the dose of the biologic drug is above a maximum dose amount. In some cases, the maximum dose amount is 80 mg when the biologic drug comprises ADA. In some cases, the maximum dose amount is 15 mg/kg when the biologic drug comprises IFX. In some cases, the maximum dose amount is 162 mg when the biologic drug comprises TCZ. In some cases, the recommendation further comprises a treatment regimen comprising a small molecule inhibitor of a Janus Kinase (JAK) or a sphingosine 1-phosphate (S1P) receptor modulator. In some cases, (a) the small molecule inhibitor of JAK comprises baricitinib, tofacitinib, or upadacitinib, or any combination thereof; and (b) wherein the SIP receptor modulator comprises fingolimod, siponimod, ozanimod, or ponesimod, or any combination thereof.

In some cases, the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some cases, the IBD comprises Crohn's disease (CD). In some cases, the IBD comprises ulcerative colitis (UC). In some cases, the subject has received a treatment comprising a current dose of the biologic drug administered to the subject at a current inter-dose interval for at least 14 contiguous weeks. In some cases, the subject has received a treatment regimen comprising a current dose of the biologic drug administered to the subject at a current inter-dose interval at least once.

In another aspect, the present disclosure provides a computer-implemented method of training an algorithm that determines an biologic drug profile of a biologic drug for a subject having an immune-mediated inflammatory disease, the method comprising: (a) receiving data from a database, wherein the data is related to a pharmacokinetic performance of the biologic drug in individuals from a reference population having the immune-mediated inflammatory disease that have been treated with the biologic drug; (b) establishing a first set of parameter estimates from the data; (c) deriving a second set of parameter estimates for a model based at least in part on the first set of parameter estimates; (d) receiving subject specific data related to the pharmacokinetic performance of the biologic drug in the subject; (e) updating the model based at least in part on the subject specific data received in (d); and (f) determining a biologic drug profile for the subject with the model, wherein the biologic drug profile comprises a dose of the biologic drug at an inter-dose interval estimated to achieve a threshold biologic drug concentration value in the subject that is sufficient to treat the immune-mediated inflammatory disease in the subject.

In some cases, the subject specific data is received from the subject by inputting the data into a mobile application on the subject's personal electronic device. In some cases, the subject specific data comprises: (a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5), measured in a biological sample obtained from the subject; (b) a weight of the subject; (c) a body mass index (BMI) of the subject; or (d) any combination of (a) to (c). In some cases, the subject specific data comprises information comprising a severity of the immune-mediated inflammatory disease or a symptom thereof. In some cases, the severity of the immune-mediated inflammatory disease comprises a disease remission, a disease recurrence, a disease type, or any combination thereof. In some cases, the severity of the symptom of the immune-mediated inflammatory disease comprises a frequency of the symptom, a type of the symptom, or a combination thereof. In some cases, the severity of the immune-mediated inflammatory disease or symptom thereof is based on a clinical disease activity index (CDAI) score. In some cases, the information about the subject comprises a weight or body mass index (BMI) of the subject. In some cases, the subject specific data is contained in one or more electronic medical records (EMRs).

In some cases, the data comprises: (a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5); (b) a weight of individuals in the reference population; (c) a body mass index (BMI) of the individuals in the reference population; or (d) any combination of (a) to (c). In some cases, the dose of the biologic drug at the inter-dose interval is determined by estimating a clearance rate of the biologic drug in the subject based, at least in part, on the weight of the subject and the level of albumin in a biological sample of the subject. In some cases, the dose of the biologic drug at the inter-dose interval is determined by: (i) estimating a clearance rate of the biologic drug in the subject based, at least in part, on the weight of the subject and the level of albumin in a biological sample of the subject; and (ii) determining whether the subject has a poor prognostic factor of pharmacokinetic origin (PPFPK), wherein the PPFPK is determined based, at least in part, on a level of the biologic drug in the biological sample of the subject and the clearance rate. In some cases, the data comprises information comprising a severity of the immune-mediated inflammatory disease or a symptom thereof. In some cases, the severity of the immune-mediated inflammatory disease comprises a disease remission, a disease recurrence, a disease type, or any combination thereof. In some cases, the severity of the symptom of the immune-mediated inflammatory disease comprises a frequency of the symptom, a type of the symptom, or a combination thereof. In some cases, the severity of the immune-mediated inflammatory disease or symptom thereof is based on a clinical disease activity index (CDAI) score. In some cases, the information about the subject comprises a weight or body mass index (BMI) of the subject. In some cases, the clinical laboratory data is contained in one or more electronic medical records (EMRs).

In some cases, the first set of parameter estimates comprises: (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. In some cases, the second set of parameter estimates comprises: (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. In some cases, the algorithm comprises a Naïve Bayes classifier algorithm. In some cases, the algorithm comprises a non-linear mixed effects model (NLME). In some cases, the algorithm comprises a Metropolis Hastings algorithm. In some cases, the dose and inter-dose interval are estimated to achieve the threshold biologic drug concentration value for the subject with a probability that is greater than a 50%. In some cases, the dose and inter-dose interval are estimated to achieve the threshold biologic drug concentration value for the subject with a probability that is between about 50% and 90%. In some cases, the dose and inter-dose interval are estimated to achieve the threshold biologic drug concentration value for the subject with a probability that is greater than or equal to about a 90%.

In some cases, the biologic drug comprises an antibody or antigen-binding fragment thereof. In some cases, the antibody comprises a monoclonal antibody. In some cases, the biologic drug comprises adalimumab (ADA). In some cases, the dose of the ADA is 40 mg and the inter-dose interval is every two weeks. In some cases, the dose of the ADA is less than or equal to about 80 mg and the inter-dose interval is greater than or equal to about every week. In some cases, the biologic drug comprises infliximab (IFX). In some cases, the dose of the IFX is 5 mg/kg and the inter-dose interval is every eight weeks. In some cases, the dose of the IFX is less than or equal to about 15 mg/kg and the inter-dose interval is greater than or equal to about every four weeks. In some cases, the biologic drug comprises tocilizumab (TCZ). In some cases, the dose of the TCZ is 162 mg and the inter-dose interval is every two weeks. In some cases, the dose of the TCZ is less than or equal to about 162 mg and the inter-dose interval is greater than or equal to about twice every week. In some cases, the threshold biologic drug concentration value comprises between about 1 mg/L and 10 mg/L. In some cases, the threshold biologic drug concentration value comprises about 5 to 10 mg/L when the biologic drug is ADA. In some cases, the threshold biologic drug concentration value comprises about 5 to 10 mg/L when the biologic drug is IFX. In some cases, the threshold biologic drug concentration value comprises about 1 to 7.5 mg/L when the biologic drug is TCZ.

In some cases, further comprising providing a recommendation to discontinue treatment of the immune-mediated inflammatory disease with the biologic drug if the dose of the biologic drug is above a maximum dose amount. In some cases, the maximum dose amount is 80 mg when the biologic drug comprises ADA. In some cases, the maximum dose amount is 15 mg/kg when the biologic drug comprises IFX. In some cases, the maximum dose amount is 162 mg when the biologic drug comprises TCZ. In some cases, the recommendation further comprises a treatment regimen comprising a small molecule inhibitor or a Janus Kinase (JAK) or a sphingosine 1-phosphate (S1P) receptor modulator. In some cases, (a) the small molecule inhibitor of JAK comprises baricitinib, tofacitinib, or upadacitinib, or any combination thereof; and (b) wherein the SIP receptor modulator comprises fingolimod, siponimod, ozanimod, or ponesimod, or any combination thereof.

In some cases, the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some cases, the IBD comprises Crohn's disease (CD). In some cases, the IBD comprises ulcerative colitis (UC). In some cases, the subject has received a treatment comprising a current dose of the biologic drug administered to the subject at a current inter-dose interval for at least 14 contiguous weeks. In some cases, the subject has received a treatment regimen comprising a current dose of the biologic drug administered to the subject at a current inter-dose interval at least once.

In some further aspect, the present disclosure provides a computer-implemented system for achieving a threshold biologic drug concentration value in a subject, the computer-implemented system comprising: a computing device comprising at least one processor; an operating system configured to perform executable instructions; a memory; and a computer program including instructions executable by the computing device to create an application comprising: a software module configured to initialize a model of a biologic drug concentration profile for a biologic drug, wherein the model comprises data related to a pharmacokinetic performance of the biologic drug in individuals from a reference population having the immune-mediated inflammatory disease that have been treated with the biologic drug; a software module configured to establish a first set of parameter estimates from the data; a software module configured to derive a second set of parameter estimates for a model based at least in part on the first set of parameter estimates; a software module configured to receive subject specific data related to the pharmacokinetic performance of the biologic drug in the subject; and a software module configured to update the model based at least in part on the subject specific data; and a software module configured to determine a biologic drug profile for the subject, wherein the biologic drug profile comprises a dose of the biologic drug at an inter-dose interval estimated to achieve a threshold biologic drug concentration value in the subject that is sufficient to treat the immune-mediated inflammatory disease in the subject.

In some cases, the subject specific parameters comprise information about the subject. In some cases, the data is received from the subject via a mobile application on a personal electronic device of the subject. In some cases, the information about the subject comprises a severity of the immune-mediated inflammatory disease or a symptom thereof. In some cases, the severity of the immune-mediated inflammatory disease comprises a disease remission, a disease recurrence, a disease type, or any combination thereof. In some cases, the severity of the symptom of the immune-mediated inflammatory disease comprises a frequency of the symptom, a type of the symptom, or a combination thereof. In some cases, the severity of the immune-mediated inflammatory disease or symptom thereof is based on a clinical disease activity index (CDAI) score. In some cases, the information about the subject comprises a weight or body mass index (BMI) of the subject.

In some cases, the data is contained in one or more electronic medical records (EMRs). In some cases, the subject specific parameters comprise two or more of: (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. In some cases, the data received from the reference population comprises: (a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5); (b) a weight of individuals in the reference population; (c) a body mass index (BMI) of the individuals in the reference population; or (d) any combination of (a) to (c). In some cases, the subject specific data comprises: (a) a level of one or more analytes in a biological sample obtained from the subject, wherein the one or more analytes comprises (1) a biologic drug, (2) autoantibodies against the biologic drug, (3) albumin, (4) IL-6, (5) CRP, or (6) any combination of (1) to (5); (b) a weight of the subject;

(c) BMI of the subject; or (d) any combination of (a) to (c). In some cases, the dose of the biologic drug at the inter-dose interval to achieve the threshold biologic drug concentration value in the subject is estimated by estimating a clearance rate of the biologic drug in the subject based, at least in part, on the weight of the subject and the level of albumin in the biological sample obtained from the subject in the newly received data from the subject. In some cases, estimating the dose of the biologic drug at the inter-dose interval to achieve the threshold biologic drug concentration value in the subject further comprises determining whether the subject has a poor prognostic factor of pharmacokinetic origin (PPFPK), wherein the PPFPK is determined based, at least in part, on the level of the biologic drug in (a)(1) and the clearance rate. In some cases, the level of one or more analytes in a biological sample obtained from the subject is measured using a mobility shift assay or a solid-phase immunoassay. In some cases, the solid-phase immunoassay comprises an enzyme-linked immunoassay (ELISA). In some cases, the biological sample comprises a serum sample. In some cases, the biological sample is obtained from the subject up to 20 days following a last administration of the biologic drug to the subject.

In some cases, model comprises a trained model. In some cases, the model comprises a Bayesian assimilation. In some cases, the model comprises a non-linear mixed effects model (NLME). In some cases, the model comprises a Markov Chain Monte Carlo (MCMC) simulation. In some cases, estimating the dose of the biologic drug at the inter-dose interval is performed with greater than a 50% confidence. In some cases, estimating the dose of the biologic drug at the inter-dose interval is performed with between about 50% and 90% confidence. In some cases, estimating the dose of the biologic drug at the inter-dose interval is performed with greater than or equal to about a 90% confidence.

In some cases, the biologic drug comprises an antibody or antigen-binding fragment thereof. In some cases, the antibody comprises a monoclonal antibody. In some cases, the biologic drug comprises adalimumab (ADA). In some cases, the dose of the ADA is 40 mg and the inter-dose interval is every two weeks. In some cases, the dose of the ADA is less than or equal to about 80 mg and the inter-dose interval is greater than or equal to about every week. In some cases, the biologic drug comprises infliximab (IFX). In some cases, the dose of the IFX is 5 mg/kg and the inter-dose interval is every eight weeks. In some cases, the dose of the IFX is less than or equal to about 15 mg/kg and the inter-dose interval is greater than or equal to about every four weeks. In some cases, the biologic drug comprises tocilizumab (TCZ). In some cases, the dose of the TCZ is 162 mg and the inter-dose interval is every two weeks. In some cases, the dose of the TCZ is less than or equal to about 162 mg and the inter-dose interval is greater than or equal to about twice every week. In some cases, the threshold biologic drug concentration value comprises between about 1 mg/L and 10 mg/L. In some cases, the threshold biologic drug concentration value comprises about 5 to 10 mg/L when the biologic drug is ADA. In some cases, the threshold biologic drug concentration value comprises about 5 to 10 mg/L when the biologic drug is IFX. In some cases, the threshold biologic drug concentration value comprises about 1 to 7.5 mg/L when the biologic drug is TCZ.

In some cases, further comprising a software module configured to provide a treatment recommendation based on the dose and inter-dose interval of the biologic drug estimated to achieve the threshold biologic drug concentration value in the subject. In some cases, the treatment recommendation comprises: (a) continuing a current treatment regimen comprising a current dose of the biologic drug at a current inter-dose interval; or (b) administering a dose of the biologic drug that is lower than the current dose to the subject at an inter-dose interval that is shorter than the current inter-dose interval; provided, in either (a) or (b), that the current dose of the biologic at the inter-dose interval is estimated to achieve the pre-specified threshold concentration of the biologic drug in the subject with a probability of greater than 50%.

In some cases, the treatment recommendation comprises administering to the subject a dose of the biologic drug that is higher than a current dose of the biologic that the subject is currently receiving in an inter-dose interval that is shorter than the current inter-dose interval, provided that the current dose of the biologic at the inter-dose interval is estimated to achieve the pre-specified threshold concentration of the biologic drug in the subject with a probability of lower than or equal to about 50%. In some cases, the treatment recommendation comprises discontinuing treatment of the immune-mediated inflammatory disease with the biologic drug if the dose of the biologic drug is above a maximum dose amount. In some cases, the maximum dose amount is 80 mg when the biologic drug comprises ADA. In some cases, the maximum dose amount is 15 mg/kg when the biologic drug comprises IFX. In some cases, the maximum dose amount is 162 mg when the biologic drug comprises TCZ. In some cases, the recommendation further comprises a treatment regimen comprising a small molecule inhibitor of a Janus Kinase (JAK) or a sphingosine 1-phosphate (SIP) receptor modulator. In some cases, (a) the small molecule inhibitor of JAK comprises baricitinib, tofacitinib, or upadacitinib, or any combination thereof; and (b) wherein the SIP receptor modulator comprises fingolimod, siponimod, ozanimod, or ponesimod, or any combination thereof.

In some cases, the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some cases, the IBD comprises Crohn's disease (CD). In some cases, the IBD comprises ulcerative colitis (UC). In some cases, the subject has received a treatment comprising a current dose of the biologic drug administered to the subject at a current inter-dose interval for at least 14 contiguous weeks. In some cases, the subject has received a treatment regimen comprising a current dose of the biologic drug administered to the subject at a current inter-dose interval at least once.

In some cases, the present disclosure provides a platform comprising: the computer-implemented system provided herein and an output device operatively connected to the computing device, wherein the output device is configured to produce a test report comprising the dose of the biologic drug and the inter-dose interval estimated to achieve the threshold biologic drug concentration value in the subject.

In some aspect, the present disclosure provides a non-transitory computer-readable storage media encoded with a computer program including instructions executable by one or more processors for achieving a threshold biologic drug concentration value in a subject, wherein the instructions comprise: (a) initializing a model of a biologic drug concentration profile for a biologic drug, wherein the model comprises data received from a reference population that were or currently are being treated with the biologic drug for treatment of an immune-mediated inflammatory disease; (b) generating subject specific parameters relating to pharmacokinetic performance of the biologic drug in the subject; (c) simulating the biologic drug concentration profile for the subject based on the subject specific parameters and the data from the reference population; (d) updating the model based on newly received data from the subject in (b) and newly received data from the reference population in (a); and (e) estimating a dose of the biologic drug at an inter-dose interval to achieve the threshold biologic drug concentration value in the subject with the model updated in (d), wherein the threshold biologic drug concentration is sufficient to treat the immune-mediated inflammatory disease in the subject.

In some cases, generating the subject specific parameters comprises compiling information about the subject. In some cases, the information about the subject comprises a severity of the immune-mediated inflammatory disease or a symptom thereof. In some cases, the data is received from the subject via a mobile application on a personal electronic device of the subject. In some cases, the severity of the immune-mediated inflammatory disease comprises a disease remission, a disease recurrence, a disease type, or any combination thereof. In some cases, the severity of the symptom of the immune-mediated inflammatory disease comprises a frequency of the symptom, a type of the symptom, or a combination thereof. In some cases, the severity of the immune-mediated inflammatory disease or symptom thereof is based on a clinical disease activity index (CDAI) score. In some cases, the information about the subject comprises a weight or body mass index (BMI) of the subject. In some cases, the data is contained in one or more electronic medical records (EMRs). In some cases, the subject specific parameters comprise two or more of: (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. In some cases, the data received from the reference population comprises: (a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5); (b) a weight of individuals in the reference population; (c) a body mass index (BMI) of the individuals in the reference population; or (d) any combination of (a) to (c). In some cases, the newly received data from the subject comprises: (a) a level of one or more analytes in a biological sample obtained from the subject, wherein the one or more analytes comprises (1) a biologic drug, (2) autoantibodies against the biologic drug, (3) albumin, (4) IL-6, (5) CRP, or (6) any combination of (1) to (5); (b) a weight of the subject; (c) BMI of the subject; or (d) any combination of (a) to (c). In some cases, estimating the dose of the biologic drug at the inter-dose interval to achieve the threshold biologic drug concentration value in the subject comprises estimating a clearance rate of the biologic drug in the subject based, at least in part, on the weight of the subject and the level of albumin in the biological sample obtained from the subject in the newly received data from the subject. In some cases, estimating the dose of the biologic drug at the inter-dose interval to achieve the threshold biologic drug concentration value in the subject further comprises determining whether the subject has a poor prognostic factor of pharmacokinetic origin (PPFPK), wherein the PPFPK is determined based, at least in part, on the level of the biologic drug in (a)(1) and the clearance rate. In some cases, the level of one or more analytes in a biological sample obtained from the subject is measured using a mobility shift assay or a solid-phase immunoassay. In some cases, the solid-phase immunoassay comprises an enzyme-linked immunoassay (ELISA). In some cases, the biological sample comprises a serum sample. In some cases, the biological sample is obtained from the subject up to 20 days following a last administration of the biologic drug to the subject.

In some cases, the model comprises a trained model. In some cases, the model comprises a Bayesian assimilation. In some cases, the model comprises anon-linear mixed effects model (NLME). In some cases, the model comprises a Markov Chain Monte Carlo (MCMC) simulation. In some cases, the dose of the biologic drug at the inter-dose interval is estimated with a probability greater than a 50%. In some cases, the dose of the biologic drug at the inter-dose interval is estimated with a probability between about 50% and 90%. In some cases, the dose of the biologic drug at the inter-dose interval is estimated with a probability of greater than or equal to about a 90%.

In some cases, the biologic drug comprises an antibody or antigen-binding fragment thereof. In some cases, the antibody comprises a monoclonal antibody. In some cases, the biologic drug comprises adalimumab (ADA). In some cases, the dose of the ADA is 40 mg and the inter-dose interval is every two weeks. In some cases, the dose of the ADA is less than or equal to about 80 mg and the inter-dose interval is greater than or equal to about every week. In some cases, the biologic drug comprises infliximab (IFX). In some cases, the dose of the IFX is 5 mg/kg and the inter-dose interval is every eight weeks. In some cases, the dose of the IFX is less than or equal to about 15 mg/kg and the inter-dose interval is greater than or equal to about four weeks. In some cases, the biologic drug comprises tocilizumab (TCZ). In some cases, the dose of the TCZ is 162 mg and the inter-dose interval is every two weeks. In some cases, the dose of the TCZ is less than or equal to about 162 mg and the inter-dose interval is greater than or equal to about twice every week. In some cases, the threshold biologic drug concentration value comprises between about 1 mg/L and 10 mg/L. In some cases, the threshold biologic drug concentration value comprises about 5 to 10 mg/L when the biologic drug is ADA. In some cases, the threshold biologic drug concentration value comprises about 5 to 10 mg/L when the biologic drug is IFX. In some cases, the threshold biologic drug concentration value comprises about 1 to 7.5 mg/L when the biologic drug is TCZ.

In some cases, the instructions further comprises providing a recommendation to discontinue treatment of the immune-mediated inflammatory disease with the biologic drug if the dose of the biologic drug is above a maximum dose amount. In some cases, the maximum dose amount is 80 mg when the biologic drug comprises ADA. In some cases, the maximum dose amount is 15 mg/kg when the biologic drug comprises IFX. In some cases, the maximum dose amount is 162 mg when the biologic drug comprises TCZ. In some cases, the recommendation further comprises a treatment regimen comprising a small molecule inhibitor of a Janus Kinase (JAK) or a sphingosine 1-phosphate (SiP) receptor modulator. In some cases, (a) the small molecule inhibitor of JAK comprises baricitinib, tofacitinib, or upadacitinib, or any combination thereof; and (b) wherein the SIP receptor modulator comprises fingolimod, siponimod, ozanimod, or ponesimod, or any combination thereof.

In some cases, the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some cases, the IBD comprises Crohn's disease (CD). In some cases, the IBD comprises ulcerative colitis (UC). In some cases, the subject has received a treatment comprising a current dose of the biologic drug administered to the subject at a current inter-dose interval for at least 14 contiguous weeks. In some cases, the subject has received a treatment regimen comprising a current dose of the biologic drug administered to the subject at a current inter-dose interval at least once.

In some further aspects, the present disclosure provides a method of treating an immune-mediated inflammatory disease of a subject, the method comprising: (a) performing or having performed an immunoassay on a biological sample obtained from the subject to determine a level of albumin and a level of a biologic drug that are predictive of clinical remission of the immune-mediated inflammatory disease of the subject, wherein the subject is currently receiving the biologic drug for treatment of the immune-mediated inflammatory disease; (b) estimating a clearance rate of the biologic drug for the subject based, at least in part, on the level of albumin determined in (a) and a weight of the subject; and (c) if the level of the biologic drug is above a cutoff level in milligrams/L (mg/L) and the clearance rate is estimated to be below a threshold level of liters (L)/day, then administering a lower dose of the biologic drug to the subject or discontinuing the treatment of the immune-mediated inflammatory disease with the biologic drug; or (d) if the level of the biologic drug is below the cutoff level and the clearance rate is estimated to be above the threshold level, then administering the biologic drug to the subject in the same or higher dose than a dose of the biologic drug the subject is currently receiving, or administering a different drug to the subject, wherein the threshold level and the cutoff level are derived from an optical Youden index.

In some cases, the level of the biologic drug is between about 3 mg/L to about 10 mg/L. In some cases, the clearance rate is estimated to be between about 0.20 L/day to about 0.4 L/day. In some cases, the threshold level is about 0.25 L/day. In some cases, the cutoff level is about 10 mg/L. In some cases, the biologic drug comprises an antibody or an antigen-binding fragment. In some cases, the antibody comprises a monoclonal antibody. In some cases, the biologic drug comprises IFX. In some cases, the biologic drug comprises TCZ. In some cases, the biologic drug comprises ADA. In some cases, the different drug comprises a small molecule inhibitor of Janus Kinase (JAK) or a sphingosine 1-phosphate (S1P) receptor modulator. In some cases, (a) the small molecule inhibitor of JAK comprises baricitinib, tofacitinib, or upadacitinib, or any combination thereof; and (b) wherein the SIP receptor modulator comprises fingolimod, siponimod, ozanimod, or ponesimod, or any combination thereof.

In some cases, the immunoassay comprises an enzyme-linked immunoassay (ELISA) or a mobility shift assay. In some cases, the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm. In some cases, the IBD comprises Crohn's disease (CD). In some cases, the IBD comprises ulcerative colitis (UC).

In some cases, the estimating the clearance rate of the biologic drug of the subject comprises: (a) inputting the level of albumin in the biological sample obtained from the subject and the weight of the subject into a model, wherein the model has been trained using pharmacokinetic data from a reference population; and (b) outputting an estimated clearance rate of the biologic drug for the subject.

In some cases, the model comprises a Bayesian assimilation. In some cases, the model comprises a non-linear mixed effects model (NLME). In some cases, the model comprises a Markov Chain Monte Carlo (MCMC) simulation.

In some cases, the reference population is comprised of reference subjects with the immune-mediated inflammatory disease who have received treatment with the biologic drug for the immune-mediated inflammatory disease.

In some aspect, the present disclosure provides a method for treating a subject with a pharmaceutical, wherein the subject has been treated with the pharmaceutical using an initial dose regimen, the method comprising obtaining subject information comprising 1) the subject's weight, 2) at least one parameter measured from a biological sample of the subject; and 3) the subject's clinical remission status; using the subject information as input for an algorithm to estimate a probability of the level of pharmaceutical in the subject's blood reaching a predetermined level after implementing a different dose regimen of the pharmaceutical; and treating the subject with an adjusted dose regimen according to the probability.

In some cases, treating the subject with an adjusted dose regimen according to the probability comprises treating the subject with the same pharmaceutical if implementing the adjusted dose regimen has at least 50%, 60%, 70%, 80%, or 90% probability estimated by the algorithm to maintain the level of the pharmaceutical in the subject's blood above the predetermined level. In some cases, the different dose regimen is the same as the adjusted dose regimen. In some cases, treating the subject with an adjusted dose regimen according to the probability comprises terminating treatment of the pharmaceutical and initiating treatment with a different pharmaceutical if implementing the different dose regimen of the pharmaceutical has less than 10%, 20%, 30%, 40%, or 50% probability estimated by the algorithm to maintain the level of the pharmaceutical in the subject's blood above the predetermined level.

In some instances, the algorithm comprises a trained machine learning algorithm. In some instances, the algorithm comprises a statistical modeling algorithm. In some instances, the statistical modeling algorithm is a Markov chain Monte Carlo algorithm. In some instances, the statistical modeling algorithm is a Metropolis-Hastings algorithm. As used herein, Markov chain Monte Carlo (MCMC) methods comprise a class of algorithms for sampling from a probability distribution. By constructing a Markov chain that has the desired distribution as its equilibrium distribution, one can obtain a sample of the desired distribution by recording states from the chain. The more steps are included, the more closely the distribution of the sample matches the actual desired distribution. Various algorithms exist for constructing chains, including the Metropolis-Hastings algorithm. As used herein, the Metropolis-Hastings algorithm is a Markov chain Monte Carlo (MCMC) method for obtaining a sequence of random samples from a probability distribution from which direct sampling is difficult. This sequence can be used to approximate the distribution (e.g. to generate a histogram) or to compute an integral (e.g. an expected value). Metropolis-Hastings and other MCMC algorithms are generally used for sampling from multi-dimensional distributions, especially when the number of dimensions is high. For single-dimensional distributions, there are usually other methods (e.g. adaptive rejection sampling) that can directly return independent samples from the distribution, and these are free from the problem of autocorrelated samples that is inherent in MCMC methods.

In some cases, the at least one parameter measured from the subject's blood comprises at least one of level of the pharmaceutical, level of antibodies to the pharmaceutical, and albumin in the subject's blood. In some cases, the method further comprises assaying or having assayed a biological sample obtained from the subject to measure the at least one parameter measured from the subject's biological sample (e.g., blood, saliva, urine, spinal fluid, tissue sample, etc.). In some cases, the at least one parameter measured from the subject's blood comprises an inflammatory marker, for example, C-reactive protein (CRP) or IL-6. In some cases, the subject is suffering from an immune mediated inflammatory disease, for example, inflammatory bowel diseases, multiple sclerosis, rheumatoid arthritis, ankylosing spondylitis, lupus, plaque psoriasis, atopic dermatitis, gout, migraine, or a combination thereof. In some cases, the clinical remission status is obtained from Electronic health record (“EEMR”) data.

In some cases, the pharmaceutical is a monoclonal antibody described herein, for example, Tocilizumab, Infliximab, or Adalimumab. In some cases, the predetermined level is 1 or 5 mg/L and wherein the pharmaceutical is Tocilizumab. In some cases, the pharmaceutical is polyclonal antibody. In some cases, the pharmaceutical is a recombinant protein, for example, Interferon Beta 1a; Interferon Beta 1b, or Semaglutide. In some cases, the pharmaceutical is a small molecule drug (e.g., azathioprine, methotrexate, etc.)

In some cases, the method further comprises communicating the test result to a pharmacy benefit manager. In some cases, the adjusted dose regimen has a shortened inter-dose interval compared to the initial dose regimen, for example, at least 10%, 20%, 30%, 40%, 50%, 60%, 70%, 80%, 90% shortened inter-dose interval. In some cases, the adjusted dose regimen has an elongated inter-dose interval compared to the initial dose regimen, for example, at least 10%, 20%, 30%, 40%, 50%, 60%, 70%, 80%, 90%, 100%, 200%, 300%, 400%, 500%, 600%, 700%, 800%, 900%, or 1000% elongated inter-dose interval. In some cases, the adjusted dose regimen has an increased dose compared to the initial dose regimen, for example, at least 10%, 20%, 30%, 40%, 50%, 60%, 70%, 80%, 90%, 100%, 200%, 300%, 400%, 500%, 600%, 700%, 800%, 900%, or 1000% increased dose compared to the initial dose regimen. In some cases, the adjusted dose regimen has a decreased dose compared to the initial dose regimen, for example, at least 10%, 20%, 30%, 40%, 50%, 60%, 70%, 80%, or 90% decreased dose compared to the initial dose regimen. In some instances, the adjusted dose regimen is at least 10%, 20%, 30%, 40%, 50%, 60%, 70%, 80%, or 90% less costly compared to the initial dose regime.

In an aspect, the present disclosure provides a method for treating a patient with Adalimumab, wherein the patient is suffering from an immune mediated inflammatory disease. The method may comprise: (a) determining whether the patient has an Adalimumab level above a pre-specified threshold by: (i) obtaining or having obtained a biological sample from the patient; and (ii) performing or having performed drug quantification on the biological sample to determine if the patient has a high likelihood or a low likelihood to achieve the pre-specified threshold; (b) if the patient has a high likelihood of achieving the pre-specified threshold, then internally administering Adalimumab to the patient in a dose of 40 mg at an inter-dose interval of every three, four, five or 6 weeks, or decreasing the dose to 20 mg at an inter-dose interval of every two weeks; (c) if the patient has a low likelihood of achieving the pre-specified threshold, then internally administering Adalimumab to the patient in a dose or inter-dose interval associated with a high likelihood to achieve the pre-specified threshold; and (d) if the dose at step c) is above 80 mg at a weekly or lesser inter-dose interval, then stopping therapy, and initiating another monoclonal antibody such as tocilizumab.

In another aspect, the present disclosure provides a method for achieving a threshold Adalimumab concentration value in a patient. The method may comprise: (a) initializing a model of an Adalimumab concentration profile, the model including data received from a reference population; (b) generating patient specific parameters; (c) simulating the Adalimumab concentration profile for the patient based on the patient specific parameters and the data received from the reference population; and (d) updating the model based on newly received data from the patient and newly received data from the reference population. In some embodiments, generating patient specific parameters comprises compiling data received from user input via a phone application.

In another aspect, the present disclosure provides a method for treating a patient with an immune mediated inflammatory disease. The method may comprise: (a) obtaining a biological sample from the patient; (b) detecting an amount of Adalimumab present in the biological sample; (c) determining whether or not the amount of Adalimumab present in the sample is above a pre-specified threshold; and (d) administering an effective amount of Adalimumab to the patient based on the determination in c).

In another aspect, the present disclosure provides a computer-implemented method of training a machine learning algorithm that determines an Adalimumab profile for a patient suffering from an immune mediated inflammatory disease. The method may comprise: (a) receiving clinical laboratory data from a database comprising data collected from patients treated with Adalimumab; (b) creating a first training set comprising patient covariate data received in the clinical laboratory data; (c) training the machine learning algorithm using the first training set; (d) checking the database for newly received clinical laboratory data; € creating a second training set comprising the newly received clinical laboratory data; (f) training the machine learning algorithm using the second training set; and (g) applying the machine learning algorithm to determine an Adalimumab profile for the patient suffering from an immune mediated inflammatory disease. In some embodiments, the clinical laboratory data comprises data received from user input via a phone application.

In another aspect, the present disclosure provides a method for treating a patient with Infliximab, wherein the patient is suffering from an immune mediated inflammatory disease. The method may comprise: (a) determining whether the patient has an Infliximab level above a pre-specified threshold by: (i) obtaining or having obtained a biological sample from the patient; and (ii) performing or having performed drug quantification on the biological sample to determine if the patient has a high likelihood or a low likelihood to achieve the pre-specified threshold; (b) if the patient has a high likelihood of achieving the pre-specified threshold, then internally administering Infliximab to the patient in a dose of 5 mg/Kg at an inter-dose interval of every 8 weeks up to 10 weeks, or decreasing the dose or prolonging the inter-dose interval to maintain blood levels above the pre-specified threshold; (c) if the patient has a low likelihood of achieving the pre-specified threshold, then internally administering Infliximab to the patient in a dose or inter-dose interval associated with a high likelihood to achieve the pre-specified threshold; and (d) if the dose at step c) is above 10 mg/Kg to achieve pre-specified threshold, then stopping therapy.

In another aspect, the present disclosure provides a method for achieving a threshold Infliximab concentration value in a patient. The method may comprise: (a) initializing a model of an Infliximab concentration profile, the model including data received from a reference population; (b) generating patient specific parameters; (c) simulating the Infliximab concentration profile for the patient based on the patient specific parameters and the data received from the reference population; and (d) updating the model based on newly received data from the patient and newly received data from the reference population. In some embodiments, generating patient specific parameters comprises compiling data received from user input via a phone application.

In another aspect, the present disclosure provides a method of treating a patient with an immune mediated inflammatory disease. The method may comprise: (a) obtaining a biological sample from the patient; (b) detecting an amount of Infliximab present in the biological sample; (c) determining whether or not the amount of Infliximab present in the sample is above a pre-specified threshold; and (d) administering an effective amount of Infliximab to the patient based on the determination in c).

In another aspect, the present disclosure provides a computer-implemented method of training a machine learning algorithm that determines an Infliximab profile for a patient suffering from an immune mediated inflammatory disease. The method may comprise: (a) receiving clinical laboratory data from a database comprising data collected from patients treated with Infliximab; (b) creating a first training set comprising patient covariate data received in the clinical laboratory data; (c) training the machine learning algorithm using the first training set; (d) checking the database for newly received clinical laboratory date creating a second training set comprising the newly received clinical laboratory data; (f) training the machine learning algorithm using the second training set; and (g) applying the machine learning algorithm to determine an Infliximab profile for the patient suffering from an immune mediated inflammatory disease. In some embodiments, the clinical laboratory data comprises data received from user input via a phone application.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a computing device; in this case, a device with one or more processors, memory, storage, and a network interface.

FIG. 2 shows a non-limiting example of a web/mobile application provision system; in this case, a system providing browser-based and/or native mobile user interfaces.

FIG. 3 shows a non-limiting example of a cloud-based web/mobile application provision system; in this case, a system comprising an elastically load balanced, auto-scaling web server and application server resources as well synchronously replicated databases.

FIG. 4 shows a non-limiting example of an application on an electronic device for receiving patient information for a subject with Crohn's disease (left) and a subject with ulcerative colitis (right).

FIG. 5 is a graph showing a distribution of adalimumab (ADA) levels within a typical patient population, according to some embodiments herein.

FIG. 6 shows likelihoods of achieving a pre-specified threshold ADA concentration, according to some embodiments herein.

FIG. 7 shows likelihoods of achieving a pre-specified threshold ADA concentration in an under exposed patient, according to some embodiments herein.

FIG. 8 shows likelihoods of achieving a pre-specified threshold ADA concentration in an over exposed patient, according to some embodiments herein.

FIG. 9A-9B show a diagram of precision dosing tool for Tocilizumab (TCZ) according to some embodiments herein. FIG. 9A provides the dosing tool for TCZ for a subject that is less than 100 kilograms (Kg), according to some embodiments herein. FIG. 9B provides the dosing tool for TCZ for a subject that is 100 Kg or more, according to some embodiments herein.

FIG. 10 shows a diagram of precision dosing tool for TCZ according to some embodiments herein. Disease Activity Score-28 (DAS-28) refers to the disease activity in 28 joints, which can be populated by the Electronic health record (EMR) and transmitted to the clinical laboratory.

FIG. 11 provides a workflow of the clinical decision tool for determining the value based pricing for TCZ, accordingly to some embodiments herein.

FIGS. 12A-12B shows a diagram of the clinical decision tool according to some embodiments herein; FIG. 12A provides the left half, and FIG. 12B provides the right half.

FIG. 13A-13B shows a diagram of the clinical decision tool according to some embodiments herein. FIG. 13A provides the left half, and FIG. 13B provides the right half.

FIG. 14 shows clinical remission in three independent inflammatory bowel disease (IBD) subject populations, and illustrates that subjects within these populations with an estimated clearance rate of above 0.25 L/day are less likely to experience clinical remission as compared with like subjects with an estimated clearance rate of below 0.25 L/day.

FIG. 15 shows clinical remission in the three independent IBD subject populations from FIG. 14, and illustrates that subjects within these populations with levels of infliximab (IFX) below 10 mg/L are less likely to experience clinical remission as compared with like subjects with levels of IFX that are above 10 mg/L.

FIG. 16 shows clinical remission versus full clinical remission in combined IBD subject populations from FIG. 14 and FIG. 15 according to some embodiments herein, and illustrates that subjects within this combined population with levels of IFX below 10 mg/L and estimated clearance rates of higher than 0.25 L/d (“Poor Prognostic Factors of Pharmacokinetic Origin,” or PPFPK, of “2”), combined, are more predictive of incomplete clinical remission in these subjects as compared to the levels of IFX (PPFPK of “0”) or estimated clearance (PPFPK of “1”), alone.

FIG. 17 shows subjects with PPFPK of 0, 1, or 2 with incomplete or full remission in the three independent IBD subject populations, and illustrates that the combined PPFPK of 2 is tightly associated with incomplete remission in each study.

FIG. 18A-18C show non-limiting example test reports, which include patient information (FIG. 18A), as well as results (FIG. 18B and FIG. 18C).

FIG. 19 shows a decision threshold calculated using an optimal Youden for IFX, according to some embodiments herein.

FIG. 20 shows the percent of patients with gastrointestinal disease that have adequate disease control in the presence of PPFPK, according to some embodiments herein. The PPFPK comprises an intrinsic property of clearance above 0.294 L/day and IFX levels at the inter-dose interval below 5 mg/L.

FIG. 20 illustrates that patients with both PPFPKs (“ppf_MNT_gt_1”) are less likely to achieve disease control than patients with one just one PPFPK (“ppf_MNT_gt_0”).

FIG. 21 shows the percent of patients with one PPFPK that have disease control across multiple groups, according to some embodiments herein.

FIG. 22 shows the association between simple endoscopic score for Crohn's disease (SESCD) and the presence or absence of symptoms and inflammation according to some embodiments herein, and illustrates that superior endoscopic outcome is achieved in the absences of inflammation and symptoms.

FIG. 23 shows the probability of patients to be above a pre-specified threshold of IFX in the presences or absences of symptoms and inflammation according to some embodiments herein, and illustrates that the absences of inflammation and symptoms is associated with a higher probability of being above a pre-specified threshold.

FIG. 24 shows the association between SESCD, the presence or absence of symptoms and inflammation, and clearance of IFX according to some embodiments herein, and illustrates a superior clinical outcome associated with lower clearance of IFX.

FIG. 25 shows the probability of patients having an inflammation, symptom, or both, who have an active SESCD score (“sesdc_active”) or who have a PPFPK (“ppf_MNT_gt_0”) according to some embodiments herein, and illustrates a higher probability that inflammation and symptoms are present in patient populations.

FIG. 26 shows the relation between PPFPK and disease control during maintenance of inflammatory bowel disease (IBD) using IFX according to some embodiments herein, and illustrates disease control is associated with the absence of PPF, as well as symptoms and inflammation.

### DETAILED DESCRIPTION

Treatment of diseases, such as an immune mediated inflammatory disease, typically has a standard dosing regimen. However, these dosing regimens may be formulated using clinical data from a large patient population, and consequently there may be wide variability in patient outcomes. For example, standard dosing regimens of biologic drugs for the treatment of inflammatory bowel disease (IBD) may be ineffective in up to about half of patients. In these patients, the pharmacokinetics behavior of these biologic drugs may be malfunctioning since they may be clearing the biologic drug too fast or may be producing too many autoantibodies. Additionally, continuing with an ineffective treatment of a drug increases both the cost to the patient, as well as overall healthcare burden. Therefore, there is a need to develop a method to determine and optimize a treatment regimen for such patients.

An optimal treatment for patients may be determined by analyzing a biological sample obtained from a subject comprising biomarkers. The biomarkers may be used as proxies for factors predictive of ineffective treatment for various biologic drugs. Based on the analyzed biomarkers, a likelihood or probability associated with a patient achieving a pre-specified threshold of the biologic drug may be determined. Such likelihood or probability may then be used to maintain or change the dose or frequency of administration of the biologic drug. This individualized, optimized treatment for patients may reduce the cost of treatment to the patient, as well as reduce the overall healthcare burden globally.

Provided herein is are clinical decision tools built on a probabilistic framework that is configured to indicate high confidence in the achievement of drug exposure above pre-specified threshold commensurate with adequate disease control and the prevention of treatment failure that associates with ineffective pharmacokinetics. The clinical decision tool disclosed herein is individualized and may be integrated with other clinical laboratory dosing tools that aim at stratifying disease progression, while also monitoring the effective silencing of inflammatory pathway. The clinical decision tools disclosed herein are provided as systems and methods to optimize treatments for patients. In some embodiments, the treatments may be determined using a model. The model may comprise a statistical model, a numerical model, a machine learning model, or any combination thereof. In some embodiments, the model may comprise Bayesian assimilation. In some embodiments, the model may comprise a non-linear mixed effects (NLME) model. In some embodiments, the model may comprise Markov Chain Monte Carlo (MCMC). In some embodiments, the model may take in patient information such as weight, body mass index (BMI), levels of analytes from a biological sample, symptoms, severity of symptoms (e.g., clinical disease activity index (CDAI) score), survey data from a patient, medical history, electronic medical records (EMR), etc. The model may then construct and evaluate conditional probability distributions, which can be used to estimate a likelihood a patient achieves a pre-specified threshold of the biologic drug. Based on the likelihood, the model may evaluate elongation or shortening the inter-dose interval of the biologic drug. In some embodiments, the model may further evaluate increasing or decreasing the dose of the biologic drug. In some embodiments, the model may further evaluate administering a non-biologic drug, such as a small molecule.

The systems and methods to optimize treatment for a patient may be applied to patients with a range of diseases. In some embodiments, the disease comprises an immune-mediated inflammatory disease, such as, but not limited to, IBD, rheumatoid arthritis (RA), cytokine release syndrome, multiple sclerosis (MS), ankylosing spondylitis (AS), lupus, plaque psoriasis, atopic dermatitis, gout, or migraine. In some cases, the IBD comprises Crohn's disease (CD). In some cases, the IBD comprises ulcerative colitis (UC). In some cases, the biologic drug comprises, but is not limited to, adalimumab (ADA), infliximab (IFX), or tocilizumab (TCZ). For example, patient information, such as weight, BMI, albumin levels, autoantibody levels, etc., may be used by a model of the present disclosure to inform physician decisions on how to change the course of treatment for patients with an immune-mediated inflammatory disease, such as those described herein.

### Computer Systems

Provided herein are computer systems configured to implement methods for determining a treatment for a subject comprising a disease. In some embodiments, the disease comprises an immune-mediated inflammatory disease. In some embodiments, the disease comprises cancer. In some embodiments, a computer-implemented system provided herein achieves a threshold biologic drug concentration in a subject. In some embodiments, the system comprises a model or algorithm for determining a biologic drug profile of a biologic drug for a subject having the disease. In some embodiments, the biologic drug profile comprises a dose of the biologic drug at an inter-dose interval estimated to achieve a threshold biologic drug concentration value in the subject that is sufficient to treat the disease in the subject.

In some embodiments, the system comprises a computing device comprising at least one processor; an operating system configured to perform executable instructions; and a memory. In some embodiments, the system further comprises a computer program including instructions executable by the computing device to create an application. In some embodiments, the application comprises one or more software modules. In some embodiments, the application comprises a software module configure to initialize a model of a biologic drug concentration profile for a biologic drug. In some embodiments, the model comprises data related to a pharmacokinetic performance of the biologic drug in individuals from a reference population, as described herein, having the immune-mediated inflammatory disease that have been treated with the biologic drug. In some embodiments, the application comprises a software module configured to establish a first set of parameter estimates from the data. In some embodiments, the application comprises a software module configured to derive a second set of parameter estimates for a model based at least in part on the first set of parameter estimates. In some embodiments, the application comprises a software module configured to receive subject specific data related to the pharmacokinetic performance of the biologic drug in the subject. In some embodiments, the application comprises a software module configured to update the model based at least in part on the subject specific data. In some embodiments, the application comprises a software module configure to determine a biologic drug profile for the subject, wherein the biologic drug profile comprises a dose of the biologic drug at an inter-dose interval estimated to achieve a threshold biologic drug concentration value in the subject that is sufficient to treat the immune-mediated inflammatory disease in the subject.

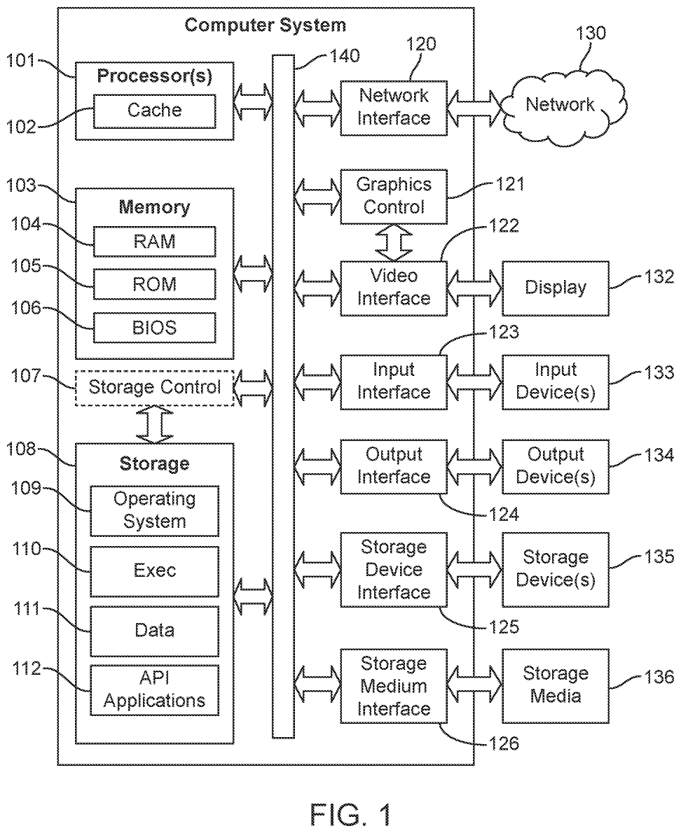

Referring to FIG. 1, a block diagram is shown depicting an exemplary machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a personal electronic device, a health tracking device, a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more steps of one or more processes described or illustrated herein. Carrying out such processes or steps may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. In some embodiments, the data 111 comprises electronic medical record (EMR) data. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. Further examples of input devices are provided herein. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

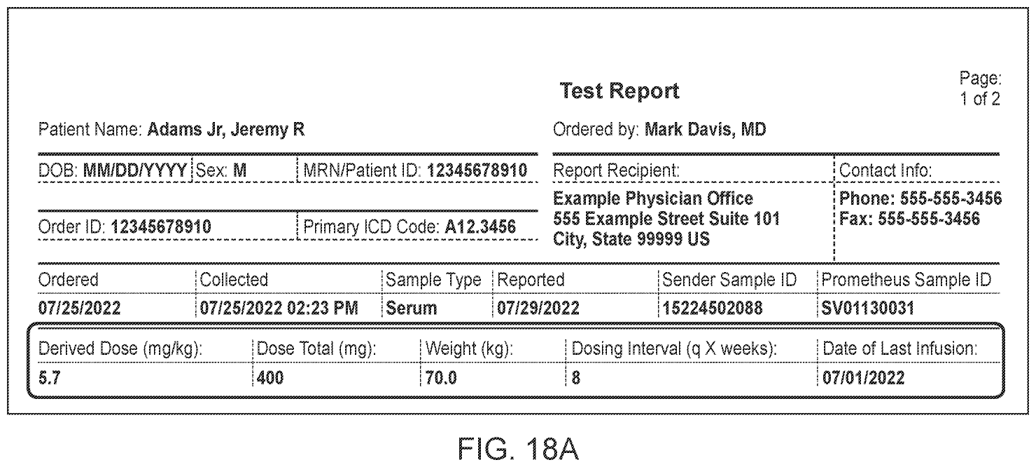

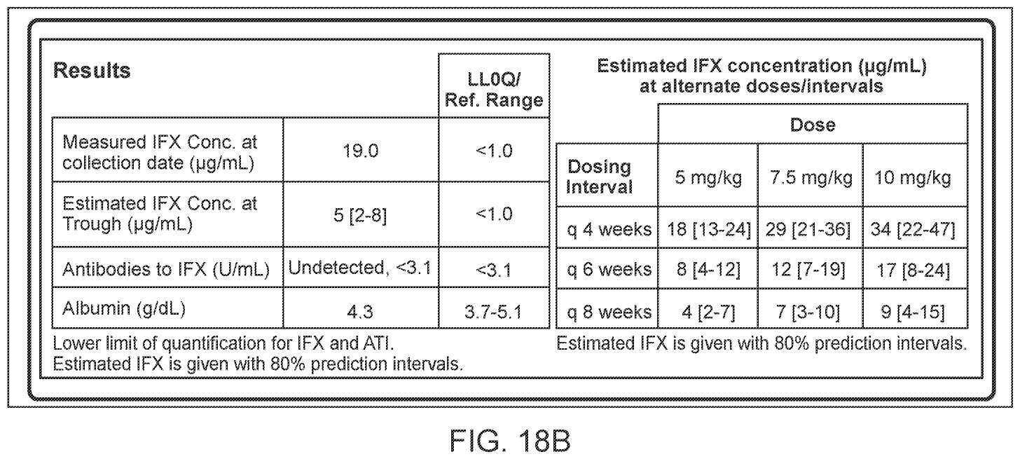

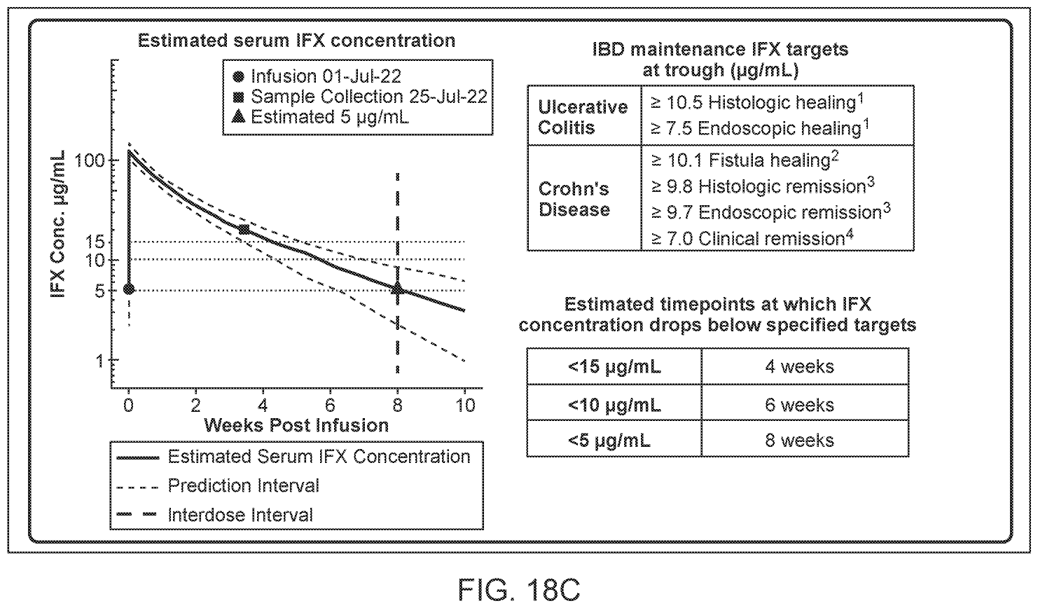

In some embodiments, the display 132 may display electronic medical record (EMR) data, user data, a treatment recommendation, facilitate communications with a user, or any combination thereof. In some embodiments, the display may facilitate communication with a user by providing a display by which a user can input information. The EMR data, user data, or input information may comprise any data related to a user's health, diet, exercise, or any combination thereof. Non-limiting examples of data are further provided herein. In some embodiments the display 132 may be communicated to user. In some embodiments, the user may comprise a patient or a subject. The patient or a subject may use the display to track their health, diet, exercise, wellbeing, or any combination thereof. In some embodiments, the user may comprise a healthcare professional, a clinician, a physician, a nurse, a pharmacist, a healthcare administrator, a technician, a veterinarian, a healthcare assistant, a therapist, a radiographer, a dentist, a surgeon, an optometrist, or any variation thereof. The healthcare professional may use the display to review, track, and/or monitor a patient's health, diet, exercise, wellbeing, or any combination thereof. The healthcare professional, for example, a physician, may further use information on the display 132 to evaluate treatments for a patient or a subject. The treatments for a patient may be evaluated based on displayed treatment recommendations and/or patient data. The healthcare professional, for example, a pharmacist, may further use information on the display 132 to fill a prescription for a patient or a subject. In some embodiments, the display 132 may display a test report, such as a test report shown in FIG. 18A-18C.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a model, method and/or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, subnotebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft Windows Mobile® OS, Linux®, and Palm® WebOS®. Those of skill in the art will also recognize that suitable media streaming device operating systems include, by way of non-limiting examples, Apple TV®, RokuR, BoxeeR, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Models

The models of the present disclosure may be implemented on a hardware, software, or combination thereof described herein. In some embodiments, a model may comprise one or more algorithms for treating a disease in a subject. The one or more algorithms may generally comprise a statistical method, a numerical method, or a machine learning method. The model may utilize one or more algorithms to analyze a biological sample obtained from a subject with a disease, such as those described herein. Analyzing a biological sample may comprise quantifying one or more analytes in a biological sample, which may include, but is not limited to, a level of biologic drug, a level of autoantibodies against the biologic drug, or both. In some embodiments, the biologic drug may comprise an antibody or an antigen-binding fragment. In some embodiments, the biologic drug comprises a monoclonal antibody, such as for example, adalimumab (ADA), infliximab (IFX), or tocilizumab (TCZ). In some embodiments, the one or more analytes further comprise albumin, C-reactive protein (CRP), interleukin 6 (IL-6), or any combination thereof. In some embodiments, the one or more analytes may comprise a biological molecule whose concentration is correlated to the concentration of the biologic drug or autoantibodies against the biologic drug.

In some embodiments, the model utilizes one or more algorithms for determining a likelihood of achieving a pre-specified threshold concentration of the biologic drug in a subject. In some embodiments, the model comprises a Markov Chain Monte Carlo (MCMC) simulation. In some embodiments, the model comprises a non-linear mixed effects (NLME) model. In some embodiments, the model comprises a machine learning model. In some embodiments, the machine learning model comprises a deep learning model. In some embodiments, the model comprises Bayesian assimilation. In some embodiments, the one or more algorithms comprises a Naive Bayes classifier algorithm. In some embodiments, the one or more algorithms comprises a Metropolis Hastings algorithm. In some embodiments, the one or more algorithms comprises an supervised, semi-supervised, or unsupervised learning algorithm. In some embodiments, the one or more algorithms comprises a clustering or a classification algorithm. In some embodiments, the one or more algorithms comprises a neural network.

In some embodiments, an algorithm may determine a likelihood of achieving a pre-specified threshold concentration of the biologic drug based, at least in part on a level of one or more analytes (e.g., biologic drug, autoantibodies, etc.) obtained from the subject. In some embodiments, an algorithm may determine a likelihood of achieving a pre-specified threshold concentration of the biologic drug based, at least in part on a current dose of the biologic drug and an current inter-dose interval of the biologic drug. The algorithm may determine the likelihood by estimating a clearance rate of the biologic drug in a subject. In some embodiments, the clearance rate is determined based, at least in part, on the weight, BMI, level of one or more analytes, or any combination thereof. The algorithm may determine the likelihood by determining whether the subject has a poor prognostic factor of pharmacokinetic origin (PPFPK). In some embodiments, the PPFPK is determined based, at least in part, on the level of a biologic drug, clearance rate, or both.

In some embodiments, the model may establish a first set of parameter estimates from a reference population. The reference population may comprise a population that has received the biologic drug for treatment of the same disease as the subject. The subject may not be part of the reference population. The first set of parameters may comprise, by way of non-limiting example, (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. In some embodiments, the model may derive a second set of parameter estimates for the model based at least in part on the first set of parameter estimates established. The second set of parameters may comprise, by way of non-limiting example, (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or ( ) log transformed covariates on the subject specific parameters in one or more of (a) to (g) as determined using non-linear mixed effect modeling; or (k) any combination thereof. In some embodiments, the model may receive input data comprising (i) the analytes quantified in the biological sample obtained from the subject and (ii) the current dose of the biologic drug and the current inter-dose interval. The model may then be interrogated based at least in part based on the data.

In some embodiments, the model as described herein may determine a biologic drug profile of a biologic drug for a subject having a disease. The biologic drug profile may comprise a dose of the biologic drug at an inter-dose interval estimated to achieve a threshold biologic drug concentration value in the subject that is sufficient to treat the disease in the subject. An algorithm for such model may be trained by receiving data from a database. The data may be related to a pharmacokinetic performance of the biologic drug in individuals from a reference population having the disease that have been treated with the biologic drug. The data may comprise a level of one or more analytes such as those described herein, weight, BMI, or any combination thereof. The database may comprise database for storing EMRs. In some embodiments, the algorithm may then establish a first set of parameter estimates from the data and further derive a second set of parameter estimates for a model based at least in part on the first set of parameter estimates, as previously described herein.

The model may then receive subject specific data related to the pharmacokinetic performance of the biologic drug in a subject. The subject specific data may be obtained by inputting the data into a mobile application on the subject's personal electronic device, such as those described herein. The subject specific data may comprise a level of one or more analytes such as those described herein, weight, BMI, or any combination thereof. The subject specific data may comprise information comprising a severity of the disease or a symptom thereof, such as, a disease remission, a disease recurrence, a disease type, a frequency of the symptom, a type of the symptom, or any combination thereof. Various non-limiting examples of symptoms of diseases are provided herein. In some embodiments, a severity of the disease or a symptom thereof may be based at least in part of an index or score. The index or score may be adjusted based at least in part on the disease, the model, the subject specific data, the reference population, or any combination thereof. In some embodiments, the index comprises a clinical disease activity index (CDAI). The subject specific data may further comprise weight, BMI, or both. In some embodiments, the subject specific data is contained in one or more electronic medical records (EMRs). The model may be updated based at least in part on the subject specific data that was received. The model may then determine a biologic drug profile for the subject.

The model trained using methods of the present disclosure may be initialized using data received from a reference population that were or currently are being treated with the biologic drug for treatment of an immune-mediated inflammatory disease. Subject specific parameters may be generated relating to pharmacokinetic performance of the biologic drug in the subject, and a biologic drug concentration profile for the subject may be simulated based on the subject specific parameters and the data from the reference population. In some embodiments, the subject specific parameters comprise two or more of (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or j) log transformed covariates on the subject specific parameters in one or more of (a) to (g) as determined using non-linear mixed effect modeling; or (k) any combination thereof. In some embodiments, the model may be updated based on newly received data from the subject, newly received data from the reference population, or both. The model may then estimate a dose of the biologic drug at an inter-dose interval to achieve the threshold biologic drug concentration value in the subject with the mode. In some cases, the threshold biologic drug concentration is sufficient to treat the immune-mediated inflammatory disease in the subject.

In some embodiments, the model estimates the dose of the biologic drug at the inter-dose interval with greater than a 50% confidence. In some embodiments, the model estimates the dose of the biologic drug at the inter-dose interval with between about 50% and 90% confidence. In some embodiments, the model estimates the dose of the biologic drug at the inter-dose interval with between about 50% to 95%, 55% to 90%, 60% to 85%, 65% to 80%, or 70% to 75% confidence. In some embodiments, the model estimates the dose of the biologic drug at the inter-dose interval with about 50%, 55%, 60%, 65%, 70%, 75%, 80%, 85%, 90%, or 95% confidence. In some embodiments, the model estimates the dose of the biologic drug at the inter-dose interval with at least about 50%, 55%, 60%, 65%, 70%, 75%, 80%, 850%, 90%, or 95% confidence. In some embodiments, the model estimates the dose of the biologic drug at the inter-dose interval with greater than or equal to about a 90% confidence.

In some embodiments, the model provides a recommendation to discontinue treatment of the disease with the biologic drug if the dose of the biologic drug is above a maximum dose amount. In some embodiments, the recommendation further comprises a treatment regimen with a small molecule, such as, but not limited to, those described herein. In some embodiments, the small molecule is a small molecule inhibitor, such as those described herein.

### Input Devices

In some embodiments, the computing system comprises a input device. The input device may be used to input user information that can be accessed on the computing system. The input device may further be used to receive subject specific information, which can be accessed on the computing system. In some embodiments, the subject of the subject specific information has a disease (e.g., cancer, an immune mediated disease, etc.). User information may comprise symptoms, symptom severity, symptom duration, weight, temperature, heart rate, etc. In some embodiments, user information is obtained from a survey displayed on an input device. In some embodiments, the input device comprises a sensor. In some embodiments, the sensor may be used to track a user's health. A sensor may include, but is not limited to, an accelerometer, a heart rate sensor, a blood pressure sensor, a blood glucose sensor, a sweat sensor a skin conductivity sensor or an imaging sensor, or a spectrometer. In some embodiments, the input device comprises a communication element (e.g., Bluetooth®) configured to transmit the recorded sensor data to the computing system.

In some embodiments, the input device comprises a personal electronic device. In some embodiments, the personal electronic device is a handheld device. A handheld device may comprise, by way of non-limiting example, a mobile device, audio device, tablet, laptop, or any of various other mobile computing device. In some embodiments, the personal electronic device is an embedded device (e.g., a glucose monitor, a pacer, etc.). In some embodiments, the personal electronic device is worn (e.g., accessories, clothing, etc.). In some embodiments, the personal electronic device is a health tracking device. A health tracking device may comprise, by way of non-limiting example, a Fitbit®, Amazfit®, Oura Ring®, Garmin®, Apple Watch®, Galaxy Watch®, Whoop®, Jawbone®, Polar®, Under Armour®@etc.

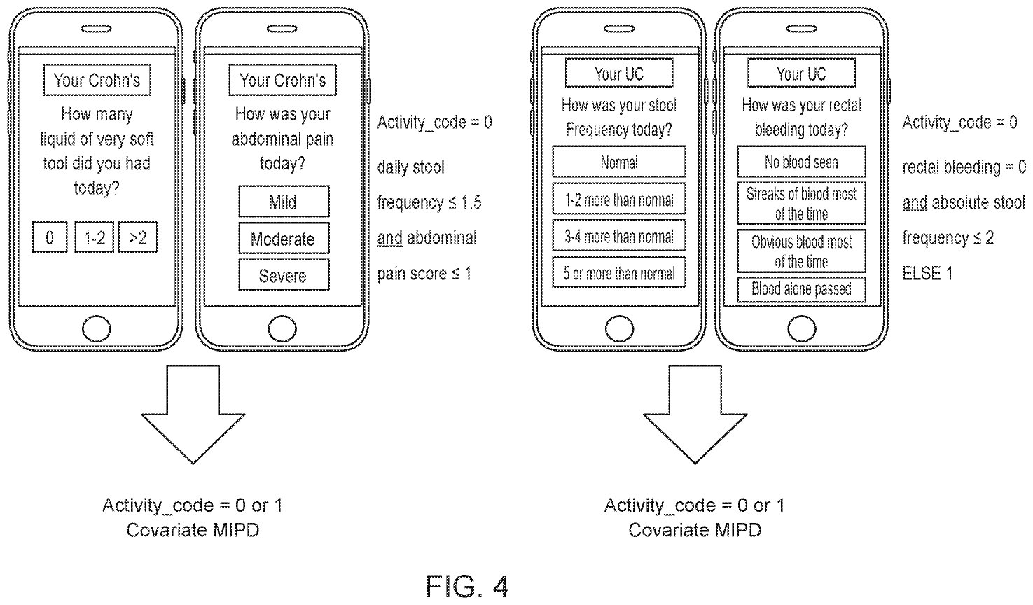

In some embodiments, the input device comprises an interface comprising a software and hardware configured to facilitate communications with the input device and the user. The hardware may comprise a display screen configured to display graphics, texts, or any other visual data. In some embodiments, the display screen is a touch screen. In some embodiments, the display screen comprises a virtual keyboard for inputting information. In alternative embodiments, a physical keyboard is used to input information. The hardware may further comprise a microphone and/or speakers to facilitate audio communications with the user. The software may comprise an application, such as those described herein. The software may be configured for a user to input information, such as clinical data or health data (e.g., symptom severity, weight, etc.). An non-limiting example of one or more user interfaces of a mobile device is provided in FIG. 4.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some embodiments, the non-transitory computer-readable storage media is encoded with a computer program including instructions executable by one or more processors for achieving a threshold biologic drug concentration value in a subject. In some embodiments, the instructions comprise: a) initializing a model of a biologic drug concentration profile for a biologic drug, b) generating subject specific parameters relating to pharmacokinetic performance of the biologic drug in the subject; c) simulating the biologic drug concentration profile for the subject based on the subject specific parameters and the data from the reference population; d) updating the model based on newly received data from the subject in (b) and newly received data from the reference population in (a); and e) estimating a dose of the biologic drug at an inter-dose interval to achieve the threshold biologic drug concentration value in the subject with the model updated in (d). In some embodiments, the model comprises data received from a reference population that were or currently are being treated with the biologic drug for treatment of an immune-mediated inflammatory disease. In some embodiments, the threshold biologic drug concentration is sufficient to treat the immune-mediated inflammatory disease in the subject.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. As such, the computer programs disclosed herein may include a sequence of instructions to perform one or more method disclosed herein. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple®QuickTime®, Microsoft® Silverlight®, Java™, and Unity®.

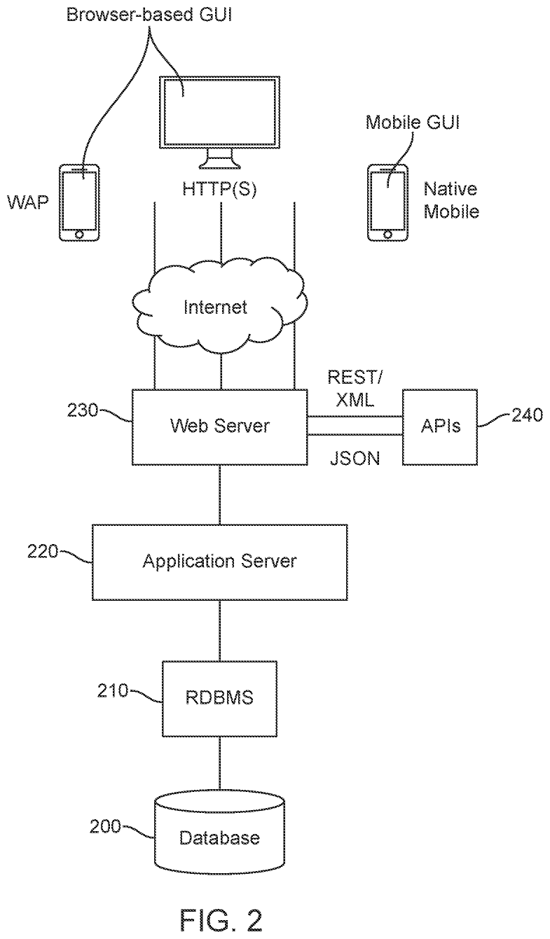

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

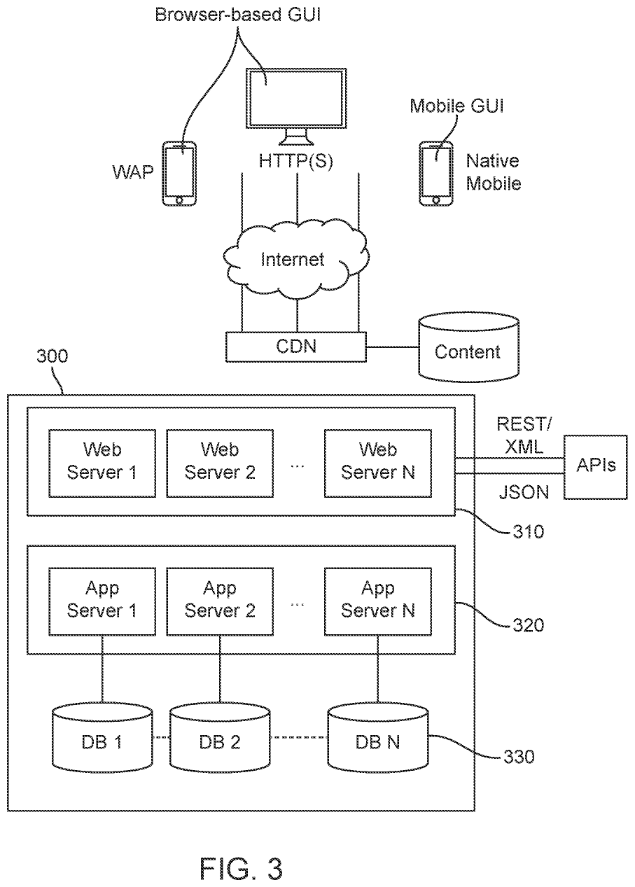

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In some embodiments, the mobile application comprises an application for tracking or logging patient health. In some embodiments, the mobile application may comprise an interface, such as, for example, the interface shown in FIG. 4. In some embodiments, the mobile application tracks a patient's symptoms of a disease, severity of a disease, diet, exercise, medications, sensor data (e.g., heart rate, glucose levels, etc.), or any other relevant patient information. In some embodiments, the mobile application comprises a survey for assessing patient wellness, such as but not limited to, those described herein. In some embodiments, the mobile application may be provided as a web application, a standalone application, or both.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. In some embodiments, the standalone application comprises the same features for tracking or logging patient health as a mobile application, as previously described herein. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

In some embodiments, an application described herein comprises one or more software modules. In some embodiments, a software module is configure to initialize a model of a biologic drug concentration profile for a biologic drug. In some embodiments, the model comprises data related to a pharmacokinetic performance of the biologic drug in individuals from a reference population, as described herein. In some embodiments, the model is a statistical model, a numerical model, a machine learning model, or any combination thereof. In some embodiments, the reference population has a disease that have been treated with the biologic drug. In some embodiments, the disease is an immune-mediated inflammatory disease. In some embodiments, the disease is cancer. In some embodiments, a software module is configured to establish a first set of parameter estimates from the data. In some embodiments, a software module is configured to derive a second set of parameter estimates for a model based at least in part on the first set of parameter estimates. In some embodiments, a software module is configured to receive subject specific data related to the pharmacokinetic performance of the biologic drug in the subject. In some embodiments, the subject specific data is received using an input device, such as those described herein. In some embodiments, a software module is configured to update the model based at least in part on the subject specific data. In some embodiments, a software module is configure to determine a biologic drug profile for the subject. In some embodiments, the biologic drug profile comprises a dose of the biologic drug at an inter-dose interval estimated to achieve a threshold biologic drug concentration value in the subject that is sufficient to treat the disease in the subject.

### Databases

In some embodiments, the platforms, systems, media, and methods disclosed herein include one or more databases, or use of the same. In some embodiments, a database comprises user information comprising user health or wellness information. In some embodiments, the user information comprises subject specific data from one or more subject comprising a disease. In some embodiments, the subject specific data comprises symptoms, symptom severity, weight, BMI, CDAI score, survey data, one or more sensor measurements as described herein, one or more analyte concentrations as those described herein, or any combination thereof. In some embodiments, the database comprises electronic medical records (EMRs). In some embodiments, the data may be accessed by a user, such as the subject. In some embodiments, the data may be accessed by a healthcare professional, such as, but not limited to, a physician, nurse, pharmacist, healthcare administrator, therapist, etc.

In view of the disclosure provided herein, those of skill in the art will recognize that many databases are suitable for storage and retrieval of subject information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

### Workflows

Further provided herein are workflows enabled by the systems disclosed herein. Exemplary workflows of using a clinical decision tool are described herein. The clinical decision tool comprises a model for determining a biologic drug profile of a biologic drug for a subject having the disease. In some embodiments, the clinical decision tool comprises a model for determining a likelihood that a threshold biologic drug concentration is achieved in a subject. In some embodiments, a clinical decision tool comprises a model for providing recommendations for a threshold biologic drug concentration in a subject.

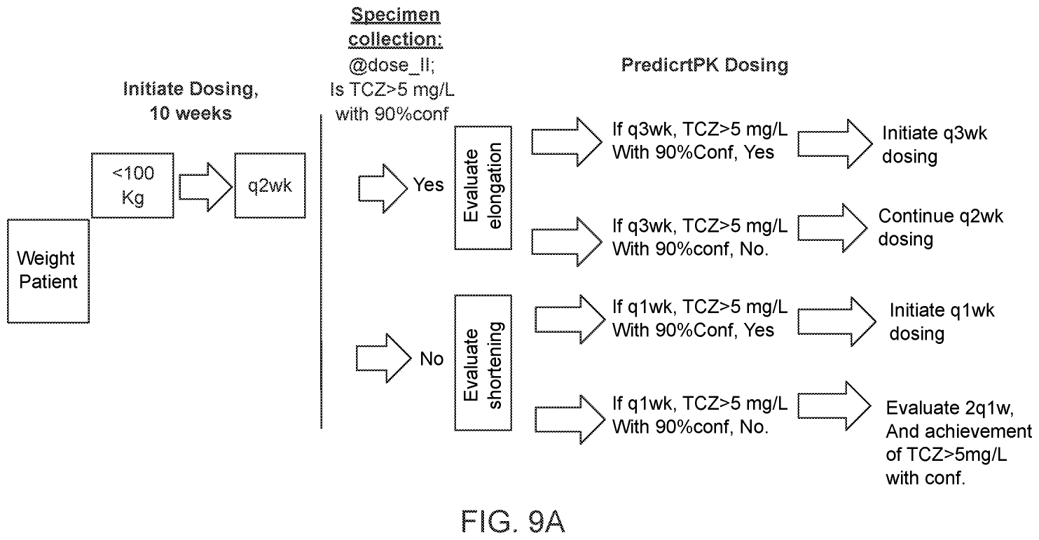

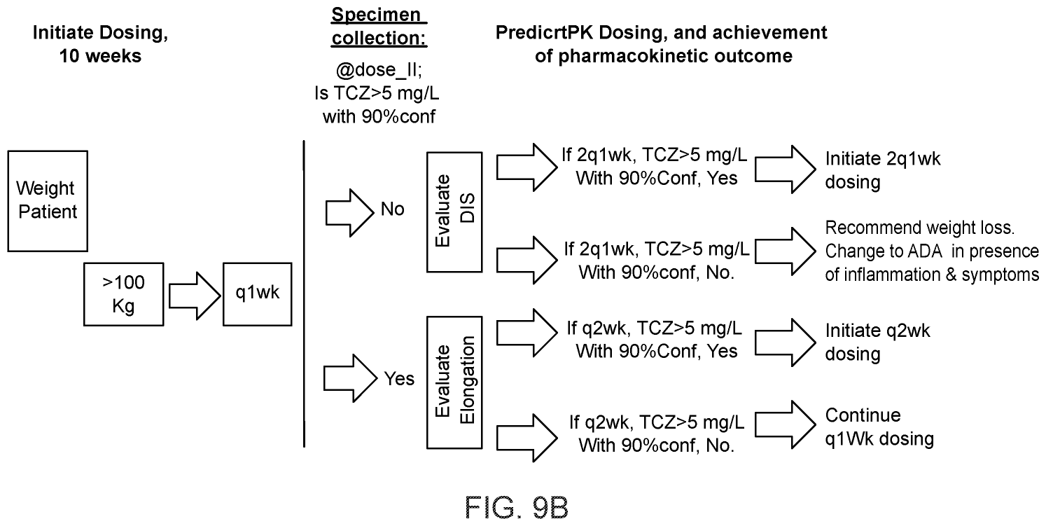

Referring to FIG. 9A, provided is an exemplary workflow for determining treatment for a subject with rheumatoid arthritis (RA) according to some embodiments herein. As shown, a patient may receive an initial dosing of a biologic drug, such as TCZ, based on initial patient information, such as the weight of the patient. For example, since a patient's weight is less than 100 kg, the subject receives an initial dosing every other week (“q2wk”) for 10 weeks. A specimen, such as the blood, may then be collected from a patient and whether the subject has a given thresholded drug concentration is evaluated with a certain likelihood. In this case, it is evaluated whether the subject has TCZ>5 mg/L with 90% confidence. If so, the subject's inter-dose interval for treatment can be elongated to every three weeks (“q3wk”). In some embodiments, a model may predict whether at an inter-dose interval of every three weeks, the subject will continue to achieve a thresholded biological drug concentration (e.g., TCZ>5 mg/L) with a certain likelihood (e.g., 90%). The model may make a prediction based on subject specific data, such as those described herein. If the output is yes, a dosing of every three weeks is initiated. If the output is no, a dosing of every other week is continued. If the subject has not achieved TCZ>5 mg/L with 90% confidence, the subject's inter-dose interval for treatment can be shortened to every week (“q1wk”). In some embodiments, a model may predict whether at an inter-dose interval of every week, the subject will continue to achieve a thresholded biological drug concentration (e.g., TCZ>5 mg/L) with a certain likelihood (e.g., 90%). If the output is yes, a dosing of every week is initiated. If the output is no, the model may further evaluate whether the inter-dose interval should be further shortened. For example, the model may evaluate whether an inter-dose interval of twice every week (“2q1w”) would achieve TCZ>5 mg/L with 90% confidence in a subject.

FIG. 9B further provides an exemplary workflow for determining treatment for a subject with rheumatoid arthritis according to some embodiments herein. As shown, a patient may receive a biologic drug every week for ten weeks as an initial dosing based on their weight of greater than 100 kg. After the ten weeks, a specimen, such as blood, is collected and whether the subject has a given thresholded drug concentration is evaluated with a certain likelihood. In this case, again, it is evaluated whether the subject has TCZ>5 mg/L with 90% confidence. If not, it is evaluated whether the subject's inter-dose interval for treatment can be shortened to twice every week. A model may predict whether at an inter-dose interval of twice every week, a subject can achieve TCZ>5 mg/L with 90% confidence. If so, a subject initiates a dosing of twice every week. If the model predicts that a subject still cannot achieve TCZ>5 mg/L with 90% confidence with an inter-dose interval of twice every week, the weight loss or another biologic drug may be recommended to the patient. In some embodiments, another biologic drug may comprise another drug for treating an immune mediate inflammatory disease, such as, for example ADA. If the subject has achieved TCZ>5 mg/L with 90% confidence, it is evaluated whether the subject's inter-dose interval can be elongated. For example, a model may evaluate whether the subject's inter-dose interval can be elongated to every other week. If yes, a dosing of every other week is initiated. If no, the subject's inter-dose interval remains once a week.

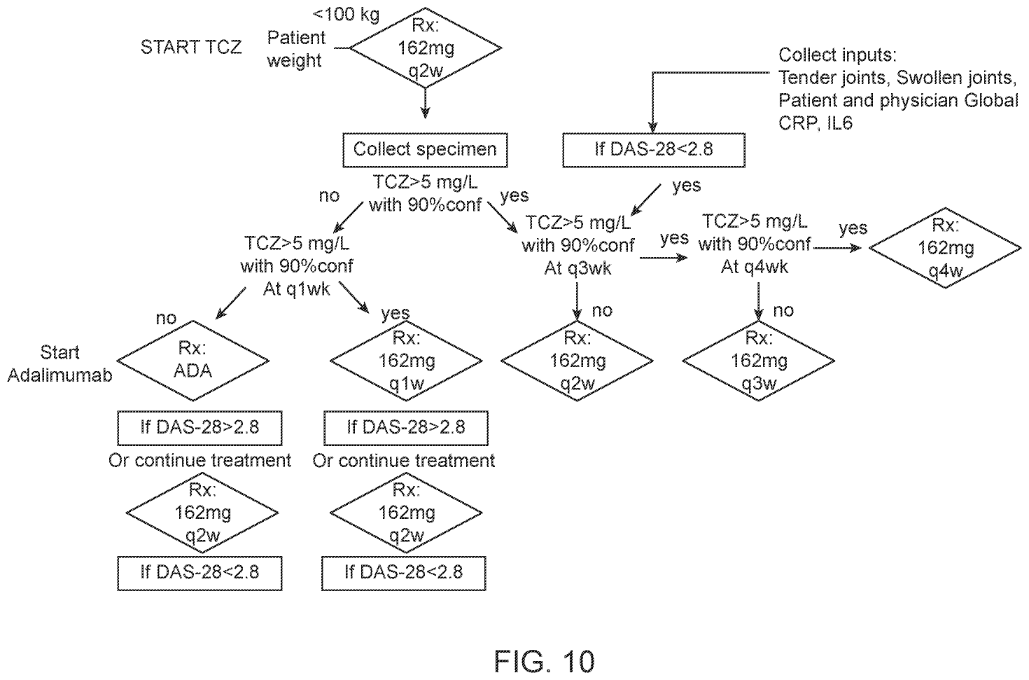

FIG. 10 provides a workflow for determining treatment for a subject with rheumatoid arthritis (RA) or cytokine release syndrome according to some embodiments herein. In this workflow, a subject's initial dosing may be determined based on their weight. If a subject's weight is less than 100 kg, a subject may receive 162 mg of TCZ every other week. After a period of time, for example, eight to twelve weeks, a specimen from a subject may be collected to evaluate whether a threshold biologic drug concentration of TCZ>5 mg/L has been achieved with 90% confidence in a subject. If not, a model is used to evaluate whether increasing the inter-dose interval to every week would achieve the threshold biologic drug concentration. In some embodiments, the course of treatment may be evaluated by the model, as well as one or more different disease assessment metrics, such as, for example disease activity score (DAS) for RA. In some embodiments, the DAS is DAS-28 for RA. DAS-28 may be determined based on collecting one or more inputs for 28 joints associated with RA. In some embodiments, the DAS is DAS-44, which may be determined based on collecting one or more inputs for 44 joints. In some embodiments, the one or more inputs may comprise tender joints or swollen joints, or a combination thereof. In some embodiments, the joints include, but are not limited to, sternoclavicular joints, acromioclavicular joints, shoulders, elbows, wrists, large knuckles, middle knucks, knees, ankles or large knuckles of the toes, or any combination thereof. In some embodiments, a DAS-28 score of more than or equal to about 5.1 indicates high disease activity. In some embodiments, a DAS-28 score of about 3.2 to about 5.1 indicates moderate disease activity. In some embodiments, a DAS-28 score of about 2.6 to about 3.2 indicates low disease activity. In some embodiments, a DAS-28 score of lower than 2.6 indicates disease remission. In some embodiments, the DAS-28 of about 2.8 is used as an input to the clinical decision tool disclosed herein. If the model determines that increasing the inter-dose interval to every week in a subject would not achieve TCZ>5 mg/L with 90% confidence, then the subject's treatment is may be switched to a different biologic drug for treating an immune mediate inflammatory disease, such as ADA. In some embodiments, the treatment may be switched to ADA if the subject has a DAS-28 of, for example, greater than 2.8. If the subject has a DAS-28 less than 2.8, the subject may continue treatment with TCZ every two weeks. If the model determines that increasing the inter-dose interval to every week in a subject would achieve TCZ>5 mg/L with 90% confidence, then the inter-dose interval may be switched to every week. In some embodiments, the treatment may be switched to every week from every two weeks if the subject has a DAS-28 of greater than 2.8. If the subject has a DAS-28 less than 2.8, the subject may continue treatment with TCZ every two weeks.

Still referring to FIG. 10, if a subject's specimen has TCZ>5 mg/L with 90% confidence, it is evaluated whether the inter-dose interval can be elongated to every three weeks. If not, the subject may continue with 162 mg of TCZ every two weeks. In some embodiments, elongation of the inter-dose interval to every three weeks is only evaluated if DAS-28<2.8. In some embodiments, if the model evaluates TCZ>5 mg/L can be achieved with 90% confidence at an inter-dose interval of every three weeks, then a further elongation of the inter-dose to every four weeks is evaluated. If a model determines TCZ>5 mg/L can be achieved with 90% confidence with an inter-dose interval of every four weeks, a subject is treated with 162 mg TCZ every four weeks. Otherwise, a subject is treated with 162 mg TCZ every three weeks.

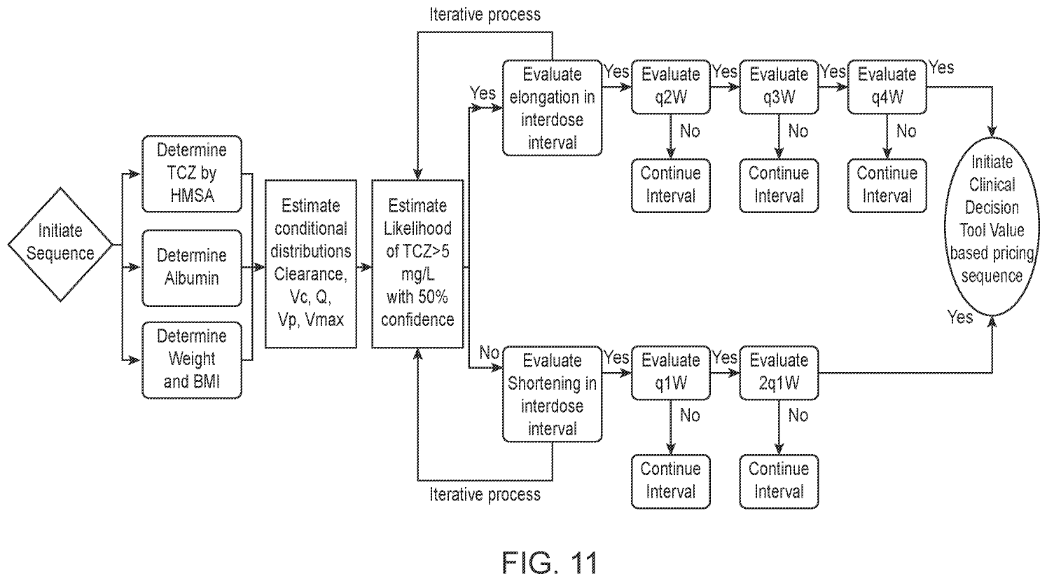

FIG. 11 provides anon-limiting workflow of the clinical decision tool for value-based pricing for TCZ according to some embodiments herein. TCZ levels and albumin are measured in a sample from the subject. TCZ levels may be measured by a homogenous mobility shift assay (HSMA). Weight and BMI are also obtained for the subject, which may be reported by a patient or may be measured by a physician or any other healthcare professional. Using these inputs, a model is then applied to estimate a plurality of conditional distributions of the parameter estimates for the subject. The parameters estimates may comprise (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. These conditional probabilities may be used to estimate a likelihood a subject achieves TCZ>5 mg/L with 50% confidence in an iterative process. For example, if there is a greater than 50% chance of achieving TCZ>5 mg/L, the clinical decision tool evaluates elongation of the inter-dose interval. As long as there continues to be a greater than 50% chance of achieving TCZ>5 mg/L, the iterative process continues to evaluate elongation of the inter-dose interval (e.g., every two weeks, every three weeks, every four weeks). Meanwhile, if there is a less than 50% change of achieving TCZ>5 mg/L, the clinical decision tool evaluates shortening of the inter-dose interval. If there continues to be a less than 50% chance of achieving TCZ>5 mg/L, the iterative process evaluates shortening the inter-dose interval (e.g., every week, twice a week). A value-based pricing sequence is then initiated for a subject based on the model prediction. In some embodiments, the value-based pricing will suggest a discounted price to reduce the costs associated with the shortening of the inter-dose interval of TCZ.

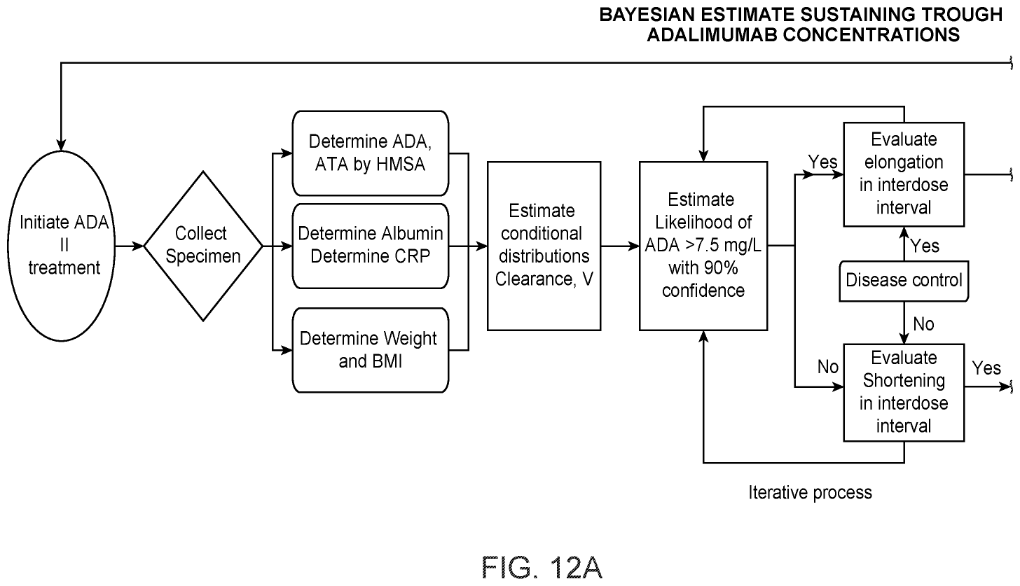

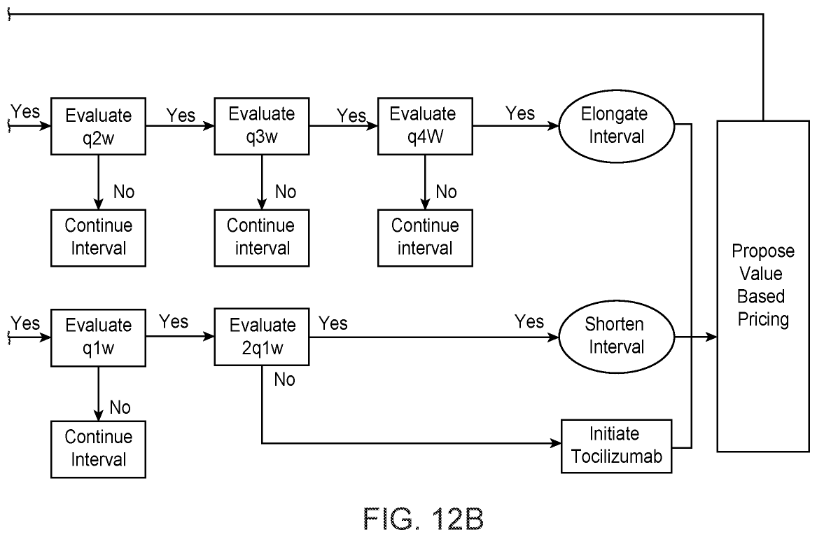

FIG. 12A-12B further provides a non-limiting workflow of the clinical decision tool for optimizing the dose and inter-dose interval of ADA according to some embodiments herein. A subject with RA may receive an initial treatment with ADA. After a period of time, a sample may be collected and a concentration of C-reactive protein, ADA levels, anti-ADA antibodies, and albumin are measured in the sample. The analytes in the sample may be measured by HSMA. Weight and BMI are also obtained for the subject, which may be reported by a patient or may be measured by a physician or any other healthcare professional. Using these inputs, a model is then applied to estimate a plurality of conditional distributions of the parameter estimates for the subject. The parameter estimates may comprise clearance and volume. These conditional probabilities may be used to estimate a likelihood a subject achieves ADA>7.5 mg/L with 90% confidence in an iterative process. For example, if there is a greater than 90% chance of achieving ADA>7.5 mg/L, the clinical decision tool evaluates elongation of the inter-dose interval. As long as there continues to be a greater than 90% chance of achieving ADA>7.5 mg/L, the iterative process continues to evaluate elongation of the inter-dose interval (e.g., every two weeks, every three weeks, every four weeks). Meanwhile, if there is a less than 90% change of achieving ADA>7.5 mg/L, the clinical decision tool evaluates shortening of the inter-dose interval. If there continues to be a less than 90% chance of achieving ADA>7.5 mg/L, the iterative process evaluates shortening the inter-dose interval (e.g., every week, twice a week). If there is less than 90% chance of achieving ADA>7.5 mg/L with an inter-dose interval of twice a week, then a patient may receive a different biologic drug for treating RA, such as TCZ. A value-based pricing sequence is then provided for subject treatment based on the model prediction. In some embodiments, the value-based pricing will suggest a discounted price to reduce the costs associated with the shortening of the inter-dose interval of ADA.

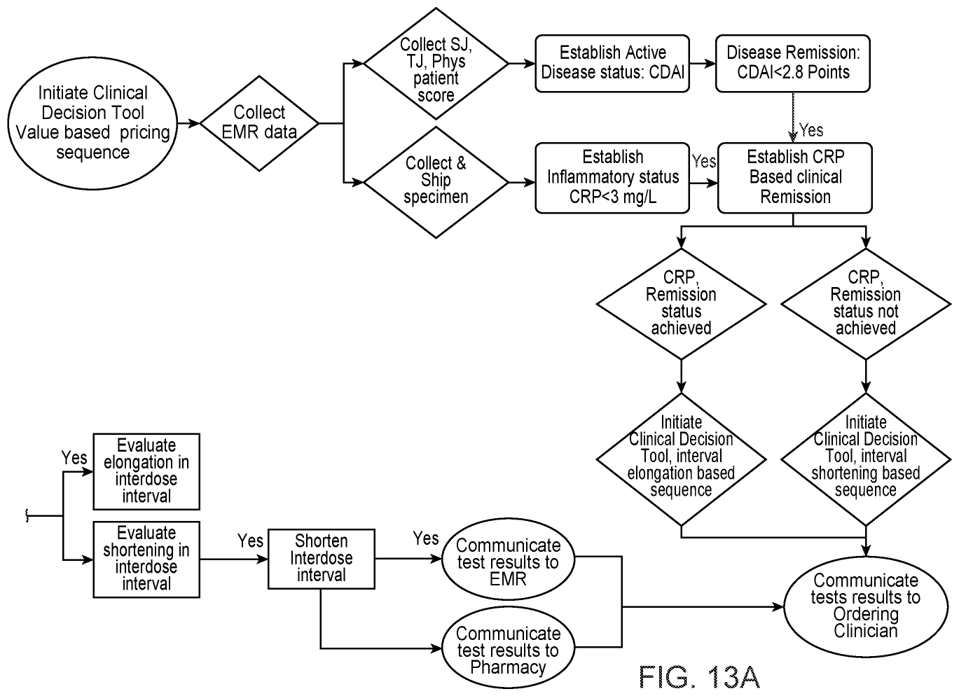

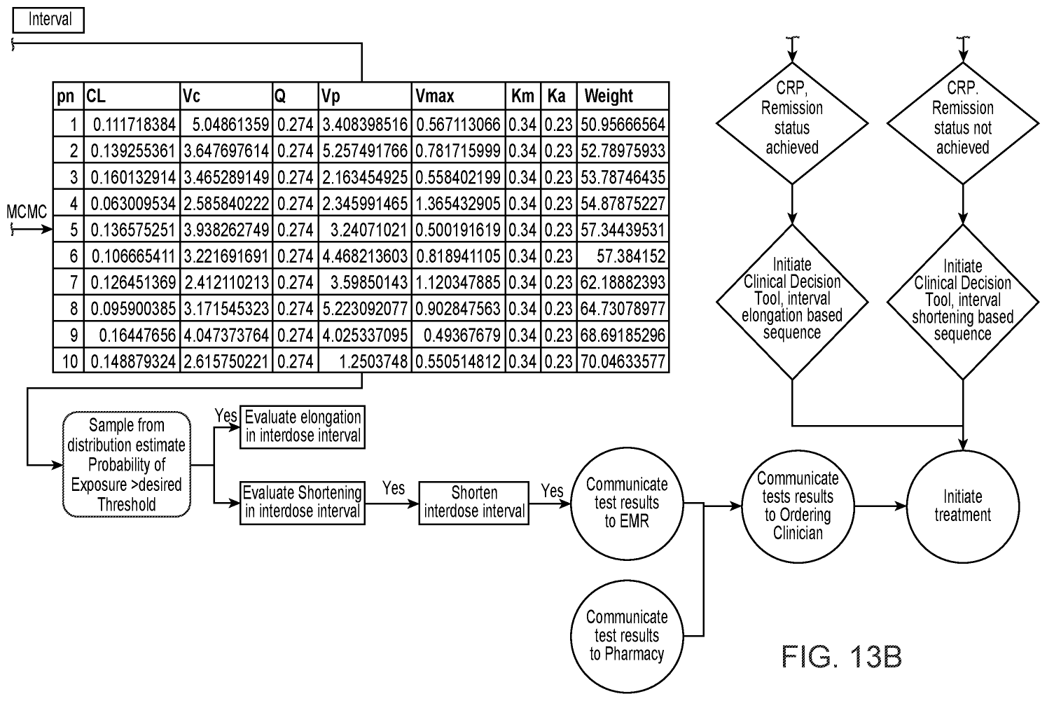

FIG. 13A-13B provides an exemplary workflow for a clinical decision tool for determining whether to maintain or elongate an inter-dose interval of a biologic drug according to some embodiments herein. A value-based pricing sequence may be initiated in a patient. A patient's electronic medical record (EMR) data may be collected. In this non-limiting example, the patient's information related to disease severity is severity of rheumatoid arthritis (RA). In some embodiments, the patient's information includes, but is not limited to a clinical disease activity index (CDAI) for RA, such as the disease activity score (DAS). In some embodiment, information such as swollen joints (SJ) or tender joints (TJ) is collected, which can be used to establish active disease status using a CDAI. In some embodiments, the joints include, but are not limited to, sternoclavicular joints, acromioclavicular joints, shoulders, elbows, wrists, large knuckles, middle knucks, knees, ankles or large knuckles of the toes, or any combination thereof. A disease remission may be categorized based on the CDAI, for example is the CDAI (DAS-28)<2.8 points. In some embodiments, the CDAI is the Crohn's disease activity index (also referred to herein as CDAI), which includes patient information such as weight, total number of stools in the last 7 days, abdominal pair, general well-being, anti-diarrhea drug use, abdominal mass, hematocrit, arthritis/arthralgias, iritis/uveitis, erythema nodosum, pyoderma gangrenosum, or aphthous stomatitis, anal fissure, fistular, or abscess, other fistula, fever/temperature over 100 degrees F., or any combination thereof. In some embodiments, CDAI scores range from 0 to 600. In some embodiments, a score of less than 150 corresponds to relative disease quiescence (remission). In some embodiments, a score of about 150 to about 219 corresponds to mildly active disease. In some embodiments, a score of about 220 to about 450 corresponds to moderately active disease. In some embodiments, a score of greater than 450 corresponds to severe disease. The EMR data may also be used to collect and ship specimens from a patient, which can be used to establish inflammatory status, for example, if CRP<3 mg/L. Either the CDAI, inflammatory status, or both can be used to establish CRP based clinical remission in a patient. If the CRP remission status has been achieved, a elongation of the inter-dose interval is initiated. If the CRP remission has not been achieved, a shortening of the inter-dose interval is initiated. In some embodiments, in either case, the new inter-dose interval based sequence may be communicated to a healthcare professional, such as a clinician. In some embodiments, in either case, the patient initiates treatment with the new inter-dose interval based sequence.

Referring to FIG. 13B, a patient data (or subject specific data) from a patient receiving treatment at an initial dosing interval is evaluated. Using these inputs as previously described herein, a model is applied to estimate a plurality of conditional distributions of the parameter estimates for the subject. The parameters estimates may comprise clearance, (a) a clearance (C); (b) a volume of distribution of a central compartment (Vc); (c) intercompartmental clearance; (d) a volume of a peripheral compartment (Vp); (e) absorption rate constant; (f) maximum velocity at high biologic drug concentrations (Vmax); (g) affinity of the biologic drug to a substrate; (h) proportional error; (i) body weight; or (j) log transformed covariates on the subject specific parameters in one or more of (a) to (g) as determined using non-linear mixed effect modeling; or (k) any combination thereof. The model may be a MCMC method, such as a Metropolis Hastings algorithm. These conditional probabilities may be used to estimate a likelihood or probability that a subject achieves exposure of a drug above a desired threshold. If the likelihood is above the desired threshold, elongation of the inter-dose interval is evaluated. If the likelihood is below the desired threshold, shortening of the inter-dose interval is evaluated. If shortening the inter-dose interval achieves the likelihood above the desired threshold, the inter-dose interval is shortened. These results may be communicated to and stored as EMR data, may be communicated to a pharmacy, or both. The results may be further communicated to a healthcare professional, such as a clinician. The results may further be used to initiate treatment at a new inter-dose interval.

### Methods

Provided herein are systems and methods for optimizing a biological therapy regimen for a subject. The biological therapy regiment may comprise treating a patient with a biologic drug or a small molecule. The subject may be a patient diagnosed with disease. In some embodiments, the disease comprises an immune mediated inflammatory disease. In some embodiments, the disease comprises a cancer. In some embodiments, the systems and methods may involve inputting patient data into a model to forecast a drug concentration level in a patient and establish a dosing regimen for maintaining a pre-specified threshold drug concentration level in the patient. The pre-specified threshold may be a target concentration level for effective treatment of the disease, such as an immune mediated inflammatory disease, in the patient.

In some embodiments described herein are methods for optimizing a treatment regimen for a patient with an immune mediated inflammatory disease. In some embodiments, the treatment regimen involves treating a patient with a pharmaceutical. In some embodiments, the pharmaceutical is a biological or targeted therapy comprising a biologic drug or small molecule. In some embodiments, the biological therapy may be a monoclonal antibody. In some embodiments, the biological therapy may be a polyclonal antibody. In some embodiments, the biological therapy may be a vaccine, blood, blood components, cells, allergens, genes, tissues, hormones, and recombinant proteins.

### Diseases

In some embodiments, the disease disclosed herein comprises an immune mediated inflammatory disease. An immune-mediated disease may comprise, by way of non-limiting example, IBD, rheumatoid arthritis (RA), cytokine release syndrome, multiple sclerosis (MS), ankylosing spondylitis (AS), lupus, plaque psoriasis, atopic dermatitis, gout, migraine, or cancer. In some cases, the IBD comprises Crohn's disease (CD). In some cases, the IBD comprises ulcerative colitis (UC). In some embodiments, the disease comprises cancer. In some embodiments, the cancer comprises bladder cancer, breast cancer, cervical cancer, colorectal cancer, gynecologic cancer, kidney cancer, head and/or neck cancer, leukemia, liver cancer, lung cancer, lymphoma, mesothelioma, myeloma, ovarian cancer, prostate cancer, skin cancer, thyroid cancer, uterine cancer, vaginal or vulvar cancer. In some embodiments, lymphoma comprises Hodgkin lymphoma or non-Hodgkin lymphoma. In some embodiments, leukemia comprises acute lymphoblastic leukemia (ALL), acute myeloid leukemia (AML), chronic lymphocytic leukemia (CLL), chronic myelogenous leukemia (CML).

### Biologic Drugs

In some embodiments, the biologic drug comprises an antibody or an antigen-binding fragment thereof. In some embodiments, the antibody comprises a monoclonal antibody. In some embodiments, the biologic drug may be Infliximab (IFX), Adalimumab (ADA), Vedolizumab CDZ), or Ustekinumab (UST). In some embodiments, the targeted biological drug or small molecule may be any of. Abatacept; Adalimumab (ADA); Alemtuzumab; Anakinra; Anti-TL1a; Apremilast; Azathioprine; Baricitinib; Belimumab; Benralizumab; Bimekizumab; Brodalumab; Canakinumab; Certolizumab pegol; Cyclosporine; Dupilumab; erenumab-aooe; Estrasimod; Etanercept; Etanercept; Etrolizumab; Filgotinib; fremanezumab-vfrm; Galcanezumab-gnlm; eptinezumab-jjmr, Golimumab; Golimumab Aria; Guselkumab; Hydroxychloroquine; Infliximab (IFX); Interferon Beta Ta; Interferon Beta 1b; Ixekizumab; Lebrikizumab; Leflunomide; Mepolizumab; Methotrexate; Mirikizumab; Mycophenolate; Natalizumab; Ocrelizumab; Ofatumumab; Ozanimod; peginterferon beta-1a; Pegloticase; reslizumab; Risankizumab-rzaa; Rituximab; Sarilumab; Secukinumab; Sulfasalazine; Tezepelumab; Tildrakizumab; Tocilizumab (TCZ); Tofacitinib; tralokinumab; Ublituximab; Upadacitinib; Ustekinumab; Vedolizumab or Semaglutide. In some embodiments, the small molecule comprises a small molecule inhibitor. In some embodiments, the small molecule inhibitor is specific to a Janus Kinase (JAK). In some embodiments, the small molecule inhibitor specific to JAK comprises baricitinib, tofacitinib, or upadacitinib, or any combination thereof. In some embodiments, the small molecule is specific to a sphingosine 1-phosphate (S1P) modulator or an SIP receptor modulator. In some embodiments, the small molecule specific to an SIP receptor modulator comprises fingolimod, siponimod, ozanimod, or ponesimod, or any combination thereof.

In some embodiments, the biologic drug may be Ado-trastuzumab emtansine (Kadcyla), Afatinib (Gilotrif), Aldesleukin (Proleukin), Alectinib (Alecensa), Alemtuzumab (Campath), Atezolizumab (Tecentriq), Avelumab (Bavencio), Axitinib (Inlyta), Belimumab (Benlysta), Belinostat (Beleodaq), Bevacizumab (Avastin), Blinatumomab (Blincyto), Bortezomib (Velcade), Bosutinib (Bosulif), Brentuximab vedotin (Adcetris), Brigatinib (Alunbrig), Cabozantinib (Cabometyx [tablet], Cometriq [capsule]), Canakinumab (Ilaris), Carfilzomib (Kyprolis), Ceritinib (Zykadia), Cetuximab (Erbitux), Cobimetinib (Cotellic), Crizotinib (Xalkori), Dabrafenib (Tafinlar), Daratumumab (Darzalex), Dasatinib (Sprycel), Denosumab (Xgeva), Dinutuximab (Unituxin), Durvalumab (Imfinzi), Elotuzumab (Empliciti), Enasidenib (Idhifa), Erlotinib (Tarceva), Everolimus (Afinitor), Gefitinib (Iressa), Ibritumomab tiuxetan (Zevalin), Ibrutinib (Imbruvica), Idelalisib (Zydelig), Imatinib (Gleevec), Ipilimumab (Yervoy), Ixazomib (Ninlaro), Lapatinib (Tykerb), Lenvatinib (Lenvima), Midostaurin (Rydapt), Necitumumab (Portrazza), Neratinib (Nerlynx), Nilotinib (Tasigna), Niraparib (Zejula), Nivolumab (Opdivo), Obinutuzumab (Gazyva), Ofatumumab (Arzerra, HuMax-CD20), Olaparib (Lynparza), Olaratumab (Lartruvo), Osimertinib (Tagrisso), Palbociclib (Ibrance), Panitumumab (Vectibix), Panobinostat (Farydak), Pazopanib (Votrient), Pembrolizumab (Keytruda), Pertuzumab (Perjeta), Ponatinib (Iclusig), Ramucirumab (Cyramza), Regorafenib (Stivarga), Ribociclib (Kisqali), Rituximab (Rituxan, Mabthera), Rituximab/hyaluronidase human (Rituxan Hycela), Romidepsin (Istodax), Rucaparib (Rubraca), Ruxolitinib (Jakafi), Siltuximab (Sylvant), Sipuleucel-T (Provenge), Sonidegib (Odomzo), Sorafenib (Nexavar), Temsirolimus (Torisel), Tocilizumab (Actemra), Tofacitinib (Xeljanz), Tositumomab (Bexxar), Trametinib (Mekinist), Trastuzumab (Herceptin), Vandetanib (Caprelsa), Vemurafenib (Zelboraf), Venetoclax (Venclexta), Vismodegib (Erivedge), Vorinostat (Zolinza), or Ziv-aflibercept (Zaltrap). In some embodiments, the patient is taking one of the above-mentioned biological therapies. In other embodiments, the patient may be taking more than one of the above-mentioned biological therapies.

### Subject

In some embodiments, subjects disclosed herein encompass mammals. The subject disclosed herein can be a mammal, such as for example a mouse, rat, guinea pig, rabbit, non-human primate, or farm animal. In some instances, the subject is human. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject is susceptible to, or is inflicted with, thiopurine toxicity, or a disease caused by thiopurine toxicity (such as pancreatitis or leukopenia). The subject may experience, or is suspected of experiencing, non-response or loss-of-response to a standard treatment (e.g., anti-TNF alpha therapy, anti-a4-b7 therapy (vedolizumab), anti-IL12p40 therapy (ustekinumab), Thalidomide, or Cytoxin).

In some embodiments, the subject has one or more diseases. The disease may be a disease disclosed herein. In some embodiments, the subject has a recursive disease. In some embodiments, the subject has a disease in clinical remission. In some embodiments, the subject has a disease that is not in clinical remission. In some embodiments, the subject is responsive to a first line therapy, such as, for example, an anti-TNF inhibitor. In some embodiments, the subject is not responsive to a first line therapy, such as, for example, an anti-TNF inhibitor.

In some embodiments, the subject has a disease that has a disease severity categorized by an index or score. In some embodiments, the disease severity is classified according to a severity of illness (SOI). In some embodiments, the disease severity is classified according to a clinical disease activity index (CDAI) or Crohn's disease activity index (CDAI). In some embodiments, the disease severity is classified by a simple disease activity index (SDAI). In some embodiments, the subject has a disease severity that is classified as remission, mildly active, moderately active, severely active, fulminant disease, or any combination thereof. In some embodiments, the severity of the disease comprises a disease remission, a disease recurrence, a disease type, or any combination thereof. In some embodiments, the severity of a symptom of the disease comprises a frequency of the symptom, a type of the symptom, or a combination thereof.

In some embodiments, the subject has one or more symptoms. A symptom may comprise, but is not limited to, fever, cold, chills, sore throat, cough, fatigue, rashes, headache, congestion, nausea, vomiting, rectal bleeding, weight loss, appetite, constipation, sweating, sneezing, wheezing, shortness of breath, high blood pressure, pain (e.g., abdominal pain, join pain, food pain, etc.), swelling (e.g., foot swelling, leg swelling, etc.), dizziness, or any combination thereof. In some embodiments, the symptom is a secondary immune mediated condition, such as, for example, eczema.

In some embodiments, the subject is a baby, child, adolescence, adult, or senior. In some embodiments, the subject is in their teens, 20s, 30s, 40s, 50s, 60s, 70s, 80s, or 90s. In some embodiments, the subject is a female or a male. In some embodiments, the subject has a smoking history. In some embodiments, the subject has an alcohol history.

In some embodiments described herein, a patient may be suffering from an immune mediated inflammatory disease. In some embodiments, the patient may be suffering from any of the immune mediated inflammatory disease as shown in Table 1. Table 1 includes non-limiting examples of therapies that may be used for treatment in each of the immune mediated inflammatory disease.

### Medical Professional

The results of the clinical decision tool disclosed herein, in some embodiments, may be communicated to a medical professional. In some embodiments, the medical professional is a doctor, nurse, physician assistant, or the like. In some embodiments, the medical professional is a gastroenterologist, dermatologist, rheumatologist, or neurologist, or a combination thereof. In some embodiments, the medical professional is a pharmacist.

### Methods of Detection

Disclosed herein are methods for analyzing biologic material in a sample to detect a presence, an absence, or a quantity of one or more analytes (e.g., nucleic acid sequence, protein, carbohydrate) in the sample. In some embodiments, the sample is obtained from a subject. In some embodiments, the analyte that is detected is the biologic drug disclosed herein, or an antibody against the biologic drug. The biologic drug may be one or more drugs provided in Table 1. In some embodiments, the analyte that is detected a target protein. In some embodiments, the target protein is albumin, C-reactive protein (CRP), interleukin 6 (IL-6), or antibodies against a biologic drug disclosed herein, or any combinations thereof. A target protein or biologic drug may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Disclosed herein are methods of detecting a presence or a level of one or more serological markers in a sample obtained from a subject. In some embodiments, the one or more serological markers comprises anti-Saccharomyces cerevisiae antibody (ASCA), an anti-neutrophil cytoplasmic antibody (ANCA), antibody against E. coli outer membrane porin protein C (anti-OmpC), anti-chitin antibody, pANCA antibody, anti-I2 antibody, and anti-Cbir1 flagellin antibody. In some embodiments, the antibodies comprises immunoglobulin A (IgA), immunoglobulin G (IgG), immunoglobulin E (IgE), or immunoglobulin M (IgM), immunoglobulin D (IgD), or a combination thereof. Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELI S A is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

In some embodiments, the analyte is a nucleic acid sequence. In some embodiments, the nucleic acid sequence comprises one or more polymorphisms. In some embodiments, the sample is assayed to measure a presence, absence, or quantity the one or more polymorphisms. In some embodiments, the polymorphism comprises a copy number variant, a single nucleotide variation, or an indel (e.g., insertion/deletion).

In some cases, the nucleic acid sequence comprises DNA. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In one embodiment, detecting the analyte is performed at the nucleic acid level by performing RNA-seq, a reverse transcriptase polymerase chain reaction (RT-PCR) or a hybridization assay with oligonucleotides that are substantially complementary to portions of cDNA molecules of the at least one biomarker gene under conditions suitable for RNA-seq, RT-PCR or hybridization and obtaining expression levels of the at least one biomarker gene.

In another embodiment, detecting the analyte is performed at the nucleic acid level by performing DNA sequencing as described herein, a polymerase chain reaction (PCR, e.g., real time PCR or quantitative PCR) and/or a hybridization assay with oligonucleotides that are substantially complementary to portions of amplified DNA molecules of the gene under conditions suitable for hybridization, thereby obtaining the genotype of the biomarker genes.

In some embodiments, detecting the analyte comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modem sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3,4-ij: 5,6,7-i‘j’]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay. PCR primers and probes can be designed with tools known and used in the art. For example, forward and reverse primers for regions containing SNPs can be designed by uploading the flanking sequences into the Thermofisher OligoPerfect Primer Designer tool.

The primer set with longest amplicon can be selected for the forward and reverse primers. Flanking sequences of SNPs can be obtained from the NCBI dbSNP database. Probes can be designed with the Thermofisher SNP genotype tool. The resulting probe design from the SNP genotype tool can be then truncated to 10-20 nucleotide flanks for the final design.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In some embodiments, methods provided herein comprise extracting nucleic acids from the sample using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiment, methods of detection comprise performing a qPCR assay, in which the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, the sample is obtained from the subject or patient indirectly or directly. In some embodiments, the sample may be obtained by the subject. In other embodiments, the sample may be obtained by a healthcare professional, such as a nurse or physician. The sample may be derived from virtually any biological fluid or tissue containing genetic information, such as blood.

### Methods for Maintaining Trough Concentration of Biologic Drug

In some embodiments described herein, is a model for use in forecasting a drug concentration level in a patient and establishing a dosing regimen in a patient to achieve a desired drug concentration level. In some embodiments, the drug may be any of the biologic drug or targeted therapies described herein. In some embodiments, the desired drug concentration level may be a pre-specified or predetermined threshold in the patient at the time of drug administration. In some embodiments, the drug administration may be an infusion cycle, or may be a dose administration, as further described herein. In some embodiments, the model may be an algorithm initialized with parameters derived from data from a reference population. In some embodiments, the algorithm is a probabilistic framework that calculates the probability of maintaining a biologic drug concentration commensurate with superior disease control. In some embodiments, this calculation is based on sampling from the conditional distribution of the parameter estimates calculated from anon-linear mixed effective modelling (NONMEM). The reference population as described herein may be referred to as a reference population. The model may derive reference population data for any of the immune mediated inflammatory diseases described herein. In some embodiments, the reference population data may include data taken from a typical population of patients suffering from the immune mediated inflammatory disease. In some embodiments, the reference population data may include patient population data including serological markers, genetic markers, general patient information (e.g., age, weight, gender, etc.), and drug concentration levels in patients being administered drugs in differing dosing regimens. In some embodiments, the model may be updated with individual parameters derived from data obtained from individual patients. In some embodiments, the individual parameters may be calculated using Bayesian data assimilation methods. In some embodiments, a sample is obtained from a patient in order to estimate individual parameters. In some embodiments, a biological sample is obtained from the subject or patient indirectly or directly. In some instances, the sample may be obtained by the subject. In other instances, the sample may be obtained by a healthcare professional, such as a nurse or physician. The sample may be derived from virtually any biological fluid or tissue containing genetic information, such as blood.

TABLE 1
Non-Limiting Agents Indicated for Immune-Mediated Inflammatory Diseases

| Diseases | Therapeutic Agent |
|---|---|
| Inflammatory | Azathioprine; Methotrexate; Adalimumab; Infliximab; Certolizumab pegol; |
| Bowel Diseases | Golimumab; Ustekinumab; Tofacitinib |
| | Vedolizumab; Guselkumab; Natalizumab; Ozanimod; |
| | Estrasimod |
| Multiple sclerosis | Interferon Beta onea; Interferon Beta onea; Interferon Beta oneb; peginterferon |
| | beta-onea; Rituximab; Ocrelizumab; Ofatumumab; Alemtuzumab; Ublituximab |
| Rheumatoid Arthritis | Azathioprine; Methotrexate; Adalimumab; Infliximab; Certolizumab pegol; |
| | Etanercept; Etanercept biosimilar; Golimumab; Golimumab Aria; Abatacept; |
| | Cyclosporine; Tofacitinib; Sarilumab; Tocilizumab; Baricitinib; Leflunomide; |
| | Upadacitinib; Rituximab |
| Ankylosing spondylitis | Azathioprine; Methotrexate; Adalimumab ; Infliximab; Certolizumab pegol; |
| | Etanercept; Etanercept; Golimumab; Golimumab Aria; Secukinumab; |
| | Hydroxychloroquine |
| | Sulfasalazine |
| Lupus | Belimumab; Anifrolumab; Azathioprine, Methotrexate, Hydroxychloroquine, |
| | Rituximab, Mycophenolate |
| Plaque Psoriasis | Azathioprine; Methotrexate; Adalimumab; Infliximab |
| | Certolizumab pegol; Etanercept; Etanercept biosimilar |
| | Ustekinumab; Secukinumab; Ixekizumab; Brodalumab |
| | Cyclosporine; Apremilast; Tildrakizumab; Guselkumab; Risankizumab-rzaa |
| Atopic Dermatitis | Dupilumab; Mepolizumab; reslizumab ; Benralizumab; Lebrikizumab; |
| | tralokinumab |
| Gout | Pegloticase; allopurinol |
| Migraine | Galcanezumab-gnlm; fremanezumab-vfrm; erenumab-aooe, eptinezumab-jjmr |
| Cancer | Bevacizumab; cetuximab; Trastuzumab; Ipilimumab; nivolumab |
| | Pembrolizumab; Atezolizumab; Avelumab; Durvalumab |

In some embodiments, the individual parameters include one or more analytes (e.g., serological markers, genetic markers, drug concentration levels in the patient, antibodies against a drug), general patient information (e.g., age, weight, gender, etc.), and patient responses to questions related to the immune mediated inflammatory disease they have. In some embodiments, the general patient information comprises a score on the patient reported outcome (PRO) index. In some embodiments, the PRO is PRO2. In some embodiments, the general patient information comprises a sore on a patient global assessment (PGA) of disease activity. In either case, the PRO or PGA may correspond with one or more of the disease activity indexes disclosed herein (e.g., CDAI, DAS, etc.). Table 2 includes non-limiting examples of serological markers used in estimating individual parameters in the model. In some embodiments, one or more genetic markers may be used to estimate individual parameters in the model. In some embodiments, one genetic marker is used to estimate individual parameters in the model. In some embodiments, two or more genetic markers are used to estimate individual parameters in the model. In some embodiments, all of the genetic markers disclosed herein are used to estimate individual parameters in the model. In some embodiments, the one or more genetic markers comprises a single nucleotide variant (SNV). In some embodiments, the one or more genetic markers comprises an indel (insertion/deletion). In some embodiments, the SNV or indel is homozygous. In some embodiments, the SNV or indel is heterozygous. In some embodiments, the SNV is at rs396991 of Fc gamma receptor IIIa (FCGR3A). In some embodiments, the SNV at rs396991 comprises a G>A, G>C, or G>T (REV). In some embodiments, the SNV at rs396991 is at chromosome position 161544752 of chromosome 1 (Chr 1) (1: 161544752) according to GRCh38.p14. In some embodiments, the SNV is at rs1801274 of Fc Gamma Receptor IIa (FCGR2A). In some embodiments, the SNV at rs1801274 comprises a A>C or A>G. In some embodiments, the SNV at rs1801274 is at chr1:161509955 (GRCh38.p13). In some SNV is rs7195994 at FTO Alpha-Ketoglutarate Dependent Dioxygenase (FTO). In some embodiments, rs7195994 is an intron variant of FTO. In some embodiments, the SNV at rs7195994 comprises G>A or G>T. In some embodiments, the SNV at rs7195994 is at chr16:54026293 (GRCh38.p13). In some embodiments, the SNV is at rs1800629 of tumor necrosis factor (TNF). In some embodiments, the SNV is at rs1800629 is a 2 KB upstream variant of TNF. In some embodiments, the SNV is at rs1800629 comprises G>A. In some embodiments, the SNV is at rs1800629 is at chr6:31575254 (GRCh38.p13). In some embodiments, the SNV is at rs2097432 at Major Histocompatibility Complex, Class II, DQAlpha 1 (HLADQA1). In some embodiments, the SNV is at rs2097432 comprises at T>A or T>C. In some embodiments, the SNV is at rs2097432 is at chr6:32622994 (GRCh38.p13). In some embodiments, the genetic marker is a proxy genetic marker of any one for the genetic markers disclosed herein because it is in linkage disequilibrium (LD) therewith. In some embodiments, LD is determined with an r² of at least or about 0.70, 0.75, 0.80, 0.85, 0.90, or 1.0.

TABLE 2
Non-Limiting Examples of Individual Parameters Used in the Clinical Decision Tool

Covariate Module Part
of Model Parameters

| | |
|---|---|
| Serologies | Albumin, CRP, calprotectin (plasma) |
| Diagnostic | Perinuclear ANCA by IFA, ASCA IgA; ASCA IgG; Anti-OmpC; |
| Immunology | Anti-CBir1; Anti-A4-Fla2 IgG |
| and chemistry | Anti-FlaX IgG; Anti-12 |
| | serum amyloid A1 |
| | carcinoembryonic antigen-related cell adhesion molecule 1 |
| | vascular cell adhesion molecule 1 |
| | angiopoietin 1 |
| | angiopoietin 2 |
| | extracellular matrix metalloproteinase inducer |
| | matrix metalloproteinase-1 |
| | matrix metalloproteinase -2 |
| | matrix metalloproteinase -3 |
| | matrix metalloproteinase -9 |
| | Interleukin-7 |
| | transforming growth factor alpha |
| Mobile App | Pro2, patient global assessment of disease activity |

### Administration and Dosing

In some embodiments, the patient may be administered a biologic drug or small molecule, such as those described herein, through oral administration, inhalation, instillation, injection, sublingual or buccal administration, rectal administration, vaginal administration, or trans-dermal administration. In some embodiments, oral administration comprises a tablet, capsule, liquid, or mixtures thereof. In some embodiments, inhalation comprises an inhaler or nebulizer to administer the biologic drug or small molecule. In some embodiments, instillation comprises ocular, otic, or nasal administration of the biologic drug or small molecule. In some embodiments, injection comprises intravenous administration, intramuscular administration, intrathecal administration, or subcutaneous administration. In some embodiments, an injection may comprise an implantation by which a biologic drug or a small molecule can be released over a duration of time.

In some embodiments, the patient may receive a 1 mg, 5 mg, 10 mg, 20 mg, 30 mg, 40 mg, 50 mg, 60 mg, 70 mg, 80 mg, 90 mg, 100 mg, 110 mg, or 120 mg dose or higher of a biological drug. In some embodiments, the patient may receive a dose at specified dosing intervals. In some embodiments, the dosing interval may be one day, one week, two weeks, three weeks, four weeks, five weeks, six weeks, seven weeks, eight weeks, nine weeks, ten weeks, eleven weeks, twelve weeks, thirteen weeks, fourteen weeks, fifteen weeks, sixteen weeks, seventeen weeks, eighteen week, nineteen weeks, or twenty weeks or more.

In some embodiments, the patient may receive about 1 mg to about 200 mg of the biologic drug or small molecule. In some embodiments, the patient may receive about 1 mg to 5 mg, 1 mg to 10 mg, 1 mg to 20 mg, 1 mg to 30 mg, 1 mg to 40 mg, 1 mg to 50 mg, 1 mg to 60 mg, 1 mg to 70 mg, 1 mg to 80 mg, 1 mg to 90 mg, 1 mg to 100 mg, 1 mg to 110 mg, 1 mg to 120 mg, 1 mg to 130 mg, 1 mg to 140 mg, 1 mg to 150 mg, 1 mg to 160 mg, 1 mg to 162 mg, 1 mg to 165 mg, 1 mg to 170 mg, 1 mg to 180 mg, 1 mg to 190 mg, 1 mg to 200 mg, 5 mg to 10 mg, 5 mg to 20 mg, 5 mg to 30 mg, 5 mg to 40 mg, 5 mg to 50 mg, 5 mg to 60 mg, 5 mg to 70 mg, 5 mg to 80 mg, 5 mg to 90 mg, 5 mg to 100 mg, 5 mg to 110 mg, 5 mg to 120 mg, 5 mg to 130 mg, 5 mg to 140 mg, 5 mg to 150 mg, 5 mg to 160 mg, 5 mg to 162 mg, 5 mg to 165 mg, 5 mg to 170 mg, 5 mg to 180 mg, 5 mg to 190 mg, 5 mg to 200 mg, 10 mg to 20 mg, 10 mg to 30 mg, 10 mg to 40 mg, 10 mg to 50 mg, 10 mg to 60 mg, 10 mg to 70 mg, 10 mg to 80 mg, 10 mg to 90 mg, 10 mg to 100 mg, 10 mg to 110 mg, 10 mg to 120 mg, 10 mg to 130 mg, 10 mg to 140 mg, 10 mg to 150 mg, 10 mg to 160 mg, 10 mg to 162 mg, 10 mg to 165 mg, 10 mg to 170 mg, 10 mg to 180 mg, 10 mg to 190 mg, 10 mg to 200 mg, 20 mg to 30 mg, 20 mg to 40 mg, 20 mg to 50 mg, 20 mg to 60 mg, 20 mg to 70 mg, 20 mg to 80 mg, 20 mg to 90 mg, 20 mg to 100 mg, 20 mg to 110 mg, 20 mg to 120 mg, 20 mg to 130 mg, 20 mg to 140 mg, 20 mg to 150 mg, 20 mg to 160 mg, 20 mg to 162 mg, 20 mg to 165 mg, 20 mg to 170 mg, 20 mg to 180 mg, 20 mg to 190 mg, 20 mg to 200 mg, 30 mg to 40 mg, 30 mg to 50 mg, 30 mg to 60 mg, 30 mg to 70 mg, 30 mg to 80 mg, 30 mg to 90 mg, 30 mg to 100 mg, 30 mg to 110 mg, 30 mg to 120 mg, 30 mg to 130 mg, 30 mg to 140 mg, 30 mg to 150 mg, 30 mg to 160 mg, 30 mg to 162 mg, 30 mg to 165 mg, 30 mg to 170 mg, 30 mg to 180 mg, 30 mg to 190 mg, 30 mg to 200 mg, 40 mg to 50 mg, 40 mg to 60 mg, 40 mg to 70 mg, 40 mg to 80 mg, 40 mg to 90 mg, 40 mg to 100 mg, 40 mg to 110 mg, 40 mg to 120 mg, 40 mg to 130 mg, 40 mg to 140 mg, 40 mg to 150 mg, 40 mg to 160 mg, 40 mg to 162 mg, 40 mg to 165 mg, 40 mg to 170 mg, 40 mg to 180 mg, 40 mg to 190 mg, 40 mg to 200 mg, 50 mg to 60 mg, 50 mg to 70 mg, 50 mg to 80 mg, 50 mg to 90 mg, 50 mg to 100 mg, 50 mg to 110 mg, 50 mg to 120 mg, 50 mg to 130 mg, 50 mg to 140 mg, 50 mg to 150 mg, 50 mg to 160 mg, 50 mg to 162 mg, 50 mg to 165 mg, 50 mg to 170 mg, 50 mg to 180 mg, 50 mg to 190 mg, 50 mg to 200 mg, 60 mg to 70 mg, 60 mg to 80 mg, 60 mg to 90 mg, 60 mg to 100 mg, 60 mg to 110 mg, 60 mg to 120 mg, 60 mg to 130 mg, 60 mg to 140 mg, 60 mg to 150 mg, 60 mg to 160 mg, 60 mg to 162 mg, 60 mg to 165 mg, 60 mg to 170 mg, 60 mg to 180 mg, 60 mg to 190 mg, 60 mg to 200 mg, 70 mg to 80 mg, 70 mg to 90 mg, 70 mg to 100 mg, 70 mg to 110 mg, 70 mg to 120 mg, 70 mg to 130 mg, 70 mg to 140 mg, 70 mg to 150 mg, 70 mg to 160 mg, 70 mg to 162 mg, 70 mg to 165 mg, 70 mg to 170 mg, 70 mg to 180 mg, 70 mg to 190 mg, 70 mg to 200 mg, 80 mg to 90 mg, 80 mg to 100 mg, 80 mg to 110 mg, 80 mg to 120 mg, 80 mg to 130 mg, 80 mg to 140 mg, 80 mg to 150 mg, 80 mg to 160 mg, 80 mg to 162 mg, 80 mg to 165 mg, 80 mg to 170 mg, 80 mg to 180 mg, 80 mg to 190 mg, 80 mg to 200 mg, 90 mg to 100 mg, 90 mg to 110 mg, 90 mg to 120 mg, 90 mg to 130 mg, 90 mg to 140 mg, 90 mg to 150 mg, 90 mg to 160 mg, 90 mg to 162 mg, 90 mg to 165 mg, 90 mg to 170 mg, 90 mg to 180 mg, 90 mg to 190 mg, 90 mg to 200 mg, 100 mg to 110 mg, 100 mg to 120 mg, 100 mg to 130 mg, 100 mg to 140 mg, 100 mg to 150 mg, 100 mg to 160 mg, 100 mg to 162 mg, 100 mg to 165 mg, 100 mg to 170 mg, 100 mg to 180 mg, 100 mg to 190 mg, 100 mg to 200 mg, 110 mg to 120 mg, 110 mg to 130 mg, 110 mg to 140 mg, 110 mg to 150 mg, 110 mg to 160 mg, 110 mg to 162 mg, 110 mg to 165 mg, 110 mg to 170 mg, 110 mg to 180 mg, 110 mg to 190 mg, 110 mg to 200 mg, 120 mg to 130 mg, 120 mg to 140 mg, 120 mg to 150 mg, 120 mg to 160 mg, 120 mg to 162 mg, 120 mg to 165 mg, 120 mg to 170 mg, 120 mg to 180 mg, 120 mg to 190 mg, 120 mg to 200 mg, 130 mg to 140 mg, 130 mg to 150 mg, 130 mg to 160 mg, 130 mg to 162 mg, 130 mg to 165 mg, 130 mg to 170 mg, 130 mg to 180 mg, 130 mg to 190 mg, 130 mg to 200 mg, 140 mg to 150 mg, 140 mg to 160 mg, 140 mg to 162 mg, 140 mg to 165 mg, 140 mg to 170 mg, 140 mg to 180 mg, 140 mg to 190 mg, 140 mg to 200 mg, 150 mg to 160 mg, 150 mg to 162 mg, 150 mg to 165 mg, 150 mg to 170 mg, 150 mg to 180 mg, 150 mg to 190 mg, 150 mg to 200 mg, 160 mg to 162 mg, 160 mg to 165 mg, 160 mg to 170 mg, 160 mg to 180 mg, 160 mg to 190 mg, 160 mg to 200 mg, 162 mg to 165 mg, 162 mg to 170 mg, 162 mg to 180 mg, 162 mg to 190 mg, 162 mg to 200 mg, 165 mg to 170 mg, 165 mg to 180 mg, 165 mg to 190 mg, 165 mg to 200 mg, 170 mg to 180 mg, 170 mg to 190 mg, 170 mg to 200 mg, 180 mg to 190 mg, 180 mg to 200 mg, or 190 mg to 200 mg of the biologic drug or small molecule. In some embodiments, the patient may receive about 1 mg, 5 mg, 10 mg, 20 mg, 30 mg, 40 mg, 50 mg, 60 mg, 70, mg, 80 mg, 90 mg, 100 mg, 110 mg, 120 mg, 130 mg, 140 mg, 150 mg, 160 mg, 162 mg, 165 mg, 170 mg, 180 mg, 190 mg, or 200 mg of the biologic drug or small molecule. In some embodiments, the patient may receive at least about 1 mg, 5 mg, 10 mg, 20 mg, 30 mg, 40 mg, 50 mg, 60 mg, 70, mg, 80 mg, 90 mg, 100 mg, 110 mg, 120 mg, 130 mg, 140 mg, 150 mg, 160 mg, 162 mg, 165 mg, 170 mg, 180 mg, or 190 mg of the biologic drug or small molecule. In some embodiments, the patient may receive at most about 5 mg, 10 mg, 20 mg, 30 mg, 40 mg, 50 mg, 60 mg, 70, mg, 80 mg, 90 mg, 100 mg, 110 mg, 120 mg, 130 mg, 140 mg, 150 mg, 160 mg, 162 mg, 165 mg, 170 mg, 180 mg, 190 mg, or 200 mg of the biologic drug or small molecule.

In some embodiments, the patient may receive about 0.5 mg/kg to about 30 mg/kg of the biologic drug or small molecule. In some embodiments, the patient may receive about 0.5 mg/kg to about 1 mg/kg, about 0.5 mg/kg to about 5 mg/kg, about 0.5 mg/kg to about 10 mg/kg, about 0.5 mg/kg to about 15 mg/kg, about 0.5 mg/kg to about 20 mg/kg, about 0.5 mg/kg to about 25 mg/kg, about 0.5 mg/kg to about 30 mg/kg, about 1 mg/kg to about 5 mg/kg, about 1 mg/kg to about 10 mg/kg, about 1 mg/kg to about 15 mg/kg, about 1 mg/kg to about 20 mg/kg, about 1 mg/kg to about 25 mg/kg, about 1 mg/kg to about 30 mg/kg, about 5 mg/kg to about 10 mg/kg, about 5 mg/kg to about 15 mg/kg, about 5 mg/kg to about 20 mg/kg, about 5 mg/kg to about 25 mg/kg, about 5 mg/kg to about 30 mg/kg, about 10 mg/kg to about 15 mg/kg, about 10 mg/kg to about 20 mg/kg, about 10 mg/kg to about 25 mg/kg, about 10 mg/kg to about 30 mg/kg, about 15 mg/kg to about 20 mg/kg, about 15 mg/kg to about 25 mg/kg, about 15 mg/kg to about 30 mg/kg, about 20 mg/kg to about 25 mg/kg, about 20 mg/kg to about 30 mg/kg, or about 25 mg/kg to about 30 mg/kg of the biologic drug or small molecule. In some embodiments, the patient may receive about 0.5 mg/kg, about 1 mg/kg, about 5 mg/kg, about 10 mg/kg, about 15 mg/kg, about 20 mg/kg, about 25 mg/kg, or about 30 mg/kg of the biologic drug or small molecule. In some embodiments, the patient may receive at least about 0.5 mg/kg, about 1 mg/kg, about 5 mg/kg, about 10 mg/kg, about 15 mg/kg, about 20 mg/kg, or about 25 mg/kg of the biologic drug or small molecule. In some embodiments, the patient may receive at most about 1 mg/kg, about 5 mg/kg, about 10 mg/kg, about 15 mg/kg, about 20 mg/kg, about 25 mg/kg, or about 30 mg/kg of the biologic drug or small molecule.

In some embodiments, the patient may receive about the biologic drug at an inter-dose interval of about twice a week to about every sixteen weeks. In some embodiments, the patient may receive about the biologic drug at an inter-dose interval of about twice a week to about one week, about twice a week to about two weeks, about twice a week to about three weeks, about twice a week to about four weeks, about twice a week to about five weeks, about twice a week to about six weeks, about twice a week to about seven weeks, about twice a week to about eight weeks, about twice a week to about nine weeks, about twice a week to about ten weeks, about twice a week to about eleven weeks, about twice a week to about twelve weeks, about twice a week to about thirteen weeks, about twice a week to about fourteen weeks, about twice a week to about fifteen weeks, about twice a week to about sixteen weeks, about one week to about two weeks, about one week to about three weeks, about one week to about four weeks, about one week to about five weeks, about one week to about six weeks, about one week to about seven weeks, about one week to about eight weeks, about one week to about nine weeks, about one week to about ten weeks, about one week to about eleven weeks, about one week to about twelve weeks, about one week to about thirteen weeks, about one week to about fourteen weeks, about one week to about fifteen weeks, about one week to about sixteen weeks, about two weeks to about three weeks, about two weeks to about four weeks, about two weeks to about five weeks, about two weeks to about six weeks, about two weeks to about seven weeks, about two weeks to about eight weeks, about two weeks to about nine weeks, about two weeks to about ten weeks, about two weeks to about eleven weeks, about two weeks to about twelve weeks, about two weeks to about thirteen weeks, about two weeks to about fourteen weeks, about two weeks to about fifteen weeks, about two weeks to about sixteen weeks, about three weeks to about four weeks, about three weeks to about five weeks, about three weeks to about six weeks, about three weeks to about seven weeks, about three weeks to about eight weeks, about three weeks to about nine weeks, about three weeks to about ten weeks, about three weeks to about eleven weeks, about three weeks to about twelve weeks, about three weeks to about thirteen weeks, about three weeks to about fourteen weeks, about three weeks to about fifteen weeks, about three weeks to about sixteen weeks, about four weeks to about five weeks, about four weeks to about six weeks, about four weeks to about seven weeks, about four weeks to about eight weeks, about four weeks to about nine weeks, about four weeks to about ten weeks, about four weeks to about eleven weeks, about four weeks to about twelve weeks, about four weeks to about thirteen weeks, about four weeks to about fourteen weeks, about four weeks to about fifteen weeks, about four weeks to about sixteen weeks, about five weeks to about six weeks, about five weeks to about seven weeks, about five weeks to about eight weeks, about five weeks to about nine weeks, about five weeks to about ten weeks, about six weeks to about seven weeks, about six weeks to about eight weeks, about six weeks to about nine weeks, about six weeks to about ten weeks, about six weeks to about eleven weeks, about six weeks to about twelve weeks, about six weeks to about thirteen weeks, about six weeks to about fourteen weeks, about six weeks to about fifteen weeks, about six weeks to about sixteen weeks, about seven weeks to about eight weeks, about seven weeks to about nine weeks, about seven weeks to about ten weeks, about seven weeks to about eleven weeks, about seven weeks to about twelve weeks, about seven weeks to about thirteen weeks, about seven weeks to about fourteen weeks, about seven weeks to about fifteen weeks, about seven weeks to about sixteen weeks, about eight weeks to about nine weeks, about eight weeks to about ten weeks, about eight weeks to about eleven weeks, about eight weeks to about twelve weeks, about eight weeks to about thirteen weeks, about eight weeks to about fourteen weeks, about eight weeks to about fifteen weeks, about eight weeks to about sixteen weeks, about nine weeks to about ten weeks, about nine weeks to about eleven weeks, about nine weeks to about twelve weeks, about nine weeks to about thirteen weeks, about nine weeks to about fourteen weeks, about nine weeks to about fifteen weeks, about nine weeks to about sixteen weeks, about ten weeks to about eleven weeks, about ten weeks to about twelve weeks, about ten weeks to about thirteen weeks, about ten weeks to about fourteen weeks, about ten weeks to about fifteen weeks, about ten weeks to about sixteen weeks, about eleven weeks to about twelve weeks, about eleven weeks to about thirteen weeks, about eleven weeks to about fourteen weeks, about eleven weeks to about fifteen weeks, about eleven weeks to about sixteen weeks, about twelve weeks to about thirteen weeks, about twelve weeks to about fourteen weeks, about twelve weeks to about fifteen weeks, about twelve weeks to about sixteen weeks, about thirteen weeks to about fourteen weeks, about thirteen weeks to about fifteen weeks, about thirteen weeks to about sixteen weeks, about fourteen weeks to about fifteen weeks, about fourteen weeks to about sixteen weeks, or about fifteen weeks to about sixteen weeks. In some embodiments, the patient may receive about the biologic drug at an inter-dose interval of about twice a week, about one week, about two weeks, about three weeks, about four weeks, about five weeks, about six weeks, about seven weeks, about eight weeks, about nine weeks, about ten weeks, about eleven weeks, about twelve weeks, about thirteen weeks, about fourteen weeks, about fifteen weeks, or about sixteen weeks. In some embodiments, the patient may receive about the biologic drug at an inter-dose interval of at least about twice a week, about one week, about two weeks, about three weeks, about four weeks, about five weeks, about six weeks, about seven weeks, about eight weeks, about ten weeks, about eleven weeks, about twelve weeks, about thirteen weeks, about fourteen weeks, or about fifteen weeks. In some embodiments, the patient may receive about the biologic drug at an inter-dose interval of at most about one week, about two weeks, about three weeks, about four weeks, about five weeks, about six weeks, about seven weeks, about eight weeks, about nine weeks, about ten weeks, about eleven weeks, about twelve weeks, about thirteen weeks, about fourteen weeks, about fifteen weeks, or about sixteen weeks.

In some embodiments, the dose of the biologic drug is about 40 mg and the inter-dose interval is every two weeks. In some embodiments, the dose of the biologic drug is about 20 mg to about 80 mg, and the inter-dose interval is every week to every six weeks. In some embodiments, the maximum dose of the biologic drug is 80 mg and the minimum inter-dose interval is weekly. In some embodiments, the maximum dose amount is 80 mg. In some embodiments, the biologic drug is ADA.

In some embodiments, the dose of the biologic drug is about 5 mg/kg and the inter-dose interval is every eight weeks. In some embodiments, the dose of the biologic drug is about 3 mg/kg to about 15 mg/kg, and the inter-dose level is every four weeks to every twelve weeks. In some embodiments, the maximum dose of the biologic drug is about 15 mg/kg and the minimum inter-dose interval is every four weeks. In some embodiments, the maximum dose amount is 15 mg/kg. In some embodiments, the biologic drug is IFX.

In some embodiments, the dose of the biologic drug is about 162 mg and the inter-dose interval is every two weeks. In some embodiments, the dose of the biologic drug is about 162 mg and the inter-dose interval is twice a week to every six weeks. In some embodiments, the maximum dose of the biologic drug is 162 mg and the minimum inter-dose interval is twice a week. In some embodiments, the maximum dose amount is 162 mg. In some embodiments, the biologic drug is TCZ.

### Reference Population

The reference population as described herein may generally comprise a population to which an individual or subject's data is compared. In some embodiments, the reference population does not comprise a disease. In some embodiments, the reference population comprises a disease. In some embodiments, the reference population has the same disease as the individual or subject. In some embodiments, the reference population has not been treated with a biologic drug or small molecule. In some embodiments, the reference population has been treated with a biologic drug or small molecule. In some embodiments, data from the reference population comprises, by way of non-limiting example, a level or one or more analytes, a weight of individual, a BMI of the individuals, or any combination thereof. In some embodiments, the one or more analytes comprise serological markers, genetic markers, or both. In some embodiments, the one or more analytes comprise (1) a biologic drug, (2) autoantibodies against the biologic drug, (3) albumin, (4) interleukin 6 (IL-6), (5) C-Reactive Protein (CRP), or (6) any combination of (1) to (5).

In some embodiments, a reference population comprises a population that has a disease that has been treated with a biologic drug. In some embodiments, data from the reference population having a disease that have been treated with the biologic drug is used to establish a set of parameter estimates of the data. In some embodiments, the set of parameter estimates from the data are used to derive another set of parameter estimates for a model. The model may then be used to determine a biologic drug profile of a biologic drug for a subject having a disease. In non-limiting examples, the disease comprises an immune-mediated inflammatory disease, such as those described herein.

In some embodiments, a model for achieving a threshold biologic drug concentration value in a subject is initialized by data received from a reference population. In some embodiments, the reference population were or are currently being treated with a biologic drug for treatment of a disease. In some embodiments, the model simulates a biologic drug concentration profile for a subject based in part by data from the reference population. In some embodiments, the model is updated at least in part based on newly received data from the reference population.

### Subject Data

In some embodiments, subject specific data comprises drug or analyte concentration levels. In some embodiments, the drug concentration levels may be obtained from the subject at any time between dosing intervals. In some embodiments, the drug concentration levels may be obtained using homogenous mobility shift assays or solid phase assays, such as those described herein. In some embodiments, the drug concentration data may be imputed into the model.

In some embodiments, data of a subject is obtained by analyzing a biological sample. In some embodiments, the biological sample comprises a serum. In some embodiments, the biological sample comprises blood, saliva, urine, spinal fluid, tissue sample, or any other acceptable biological specimen. In some embodiments, the blood of a subject is obtained by arterial sampling, venipuncture sampling, or fingerstick sampling. In some embodiments, a biological sample is obtained via a biopsy.

In some embodiments, the biological sample is analyzed and one or more analytes in the biological sample are quantified. In some embodiments, the one or more analytes comprise a level of a biologic drug, a level of autoantibodies against the biologic drug, a level of albumin, or any combination thereof. In some embodiments, the one or more analytes further comprise a level of C-Reactive Protein (CRP). In some embodiments, the one or more analytes further comprise interleukin 6 (IL-6). In some embodiments, the one or more analytes are quantified using an assay. In some embodiments, the assay comprises a mobility shift assay or a solid-phase immunoassay. In some embodiments, the solid-phase immunoassay comprises an enzyme-linked immunoassay (ELISA). In some embodiments, the mobility shift assay comprises electrophoretic mobility shift assay (EMSA). In some embodiments, the mobility shift assay comprises homogenous mobility shift assay (HMSA).

In some embodiments, subject specific data is self-reported or assessed by a healthcare professional (e.g., clinician). In some embodiments, the subject specific data comprises weight, BMI, or both. In some embodiments, the patient responses to questions related to the disease, such as an immune mediated inflammatory disease, they are suffering with may be obtained at any time during the patient's therapy. In some embodiments, the patient responses may be received via a phone application, as shown in FIG. 2. In some embodiments, the patient responses may be received by any other input device described herein. FIG. 2 shows non-limiting examples of patient questions including: How many liquid or very soft stools did you have today?; How was your abdominal pain today?; How was your stool frequency today?; and How was your rectal bleeding today? In some embodiments, the patient responses may be recorded using a survey. In some embodiments, the survey comprises a patient health questionnaire (PHQ), such as PHQ-1, PHQ-2, PHQ-3, PHQ-4, PHQ-5, PHQ-6, PHQ-7, PHQ-8, PHQ-9, PHQ-10, PHQ-11, PHQ-12, PHQ-14, PHQ-15, etc. In some embodiments, the survey comprises a general anxiety disorder (GAD) survey, such as GAD-7.

In some embodiments, subject specific data comprises information about a severity of a disease or a symptom thereof. A symptom may comprise, but is not limited to, fever, cold, chills, sore throat, cough, fatigue, rashes, headache, congestion, nausea, vomiting, rectal bleeding, weight loss, appetite, constipation, sweating, sneezing, wheezing, shortness of breath, high blood pressure, pain (e.g., abdominal pain, join pain, food pain, etc.), swelling (e.g., foot swelling, leg swelling, etc.), dizziness, or any combination thereof. In some embodiments, the severity of the disease comprises a disease remission, a disease recurrence, a disease type, or any combination thereof. In some embodiments, the severity of the symptom of the disease comprises a frequency of the symptom, a type of the symptom, or a combination thereof. In some embodiments, the severity of the immune-mediated inflammatory disease or symptom thereof is based on a clinical disease activity index (CDAI) score or Crohn's disease activity index. In some embodiments, the information is self-reported by the subject. the information is self-reported by the subject inputting the information into a mobile application on a personal electronic device of the subject, such as those described herein. In some embodiments, the information about the subject is received by one or more electronic medical records (EMRs).

### Optimizing Treatment Regimen

Methods disclosed herein may be used to optimize a treatment regimen for a subject having a disease. In some embodiments, optimizing the treatment regimen comprises optimizing a dose of a biologic drug, frequency of a biologic drug (e.g., inter-dose interval), or a combination thereof. In some embodiments, the treatment of a disease in a subject is determined based at least in part on the output of a model such as those described herein. In some embodiments, the treatment of the disease may change based on the output of the model. In some embodiments, the inter-dose interval of a drug for treatment of a disease is changed based on the output of the model. In some embodiments, the dose of a drug for treatment of a disease is changed based on the output of the model. In some embodiments, a drug for treatment of a disease is discontinued and anew drug is administered for treatment of the disease based on the output of the model. In some embodiments, the treatment of the disease may not change based on the output of the model.

In some embodiments, the model outputs include a probability of achieving a pre-specified or predetermined threshold concentration of the drug in a patient, a likelihood of achieving the pre-specified threshold, and recommendations on a dosing regimen. In some embodiments, the model begins with the patient's current dosing regimen. For example, a patient's current dosing regimen may include receiving a 4 mg dose of a biological therapy every two weeks. In some embodiments, the model calculates the probability the patient will maintain a pre-specified or predetermined threshold concentration level of the drug in their body in the time leading up to the next dose administration, using the current dosing regimen. For example, if a patient's dosing interval is two weeks, the model calculates if the patient will maintain a pre-specified threshold concentration value by the time the patient receives their next dose in two weeks. In some embodiments, the probability of achieving the pre-specified threshold is shown as a percentage between 0% to 100%. In some embodiments, the probability of achieving a prespecified threshold is greater than 50%. In some embodiments, the probability of achieving a prespecified threshold is between about 50% to 90%. In some embodiments, the probability of achieving a prespecified threshold is greater than or equal to about 90%.

In some embodiments, the subject has received a treatment comprising a current dose of the biologic drug. In some embodiments, the current dose of the biologic drug is administered to the subject at a current inter-dose interval for at least 14 contiguous weeks. In some embodiments, the current dose of the biologic drug is administered to the subject at a current inter-dose interval for at least about 10, 11, 12, 13, 14, 15, 16, 17, 18, 19, or 20 contiguous weeks. In some embodiments, the current dose of the biologic drug is administered to the subject at a current inter-dose interval for at most about 10, 11, 12, 13, 14, 15, 16, 17, 18, 19, or 20 contiguous weeks. In some embodiments, the current dose of the biologic drug is administered to the subject at a current inter-dose interval for about 10, 11, 12, 13, 14, 15, 16, 17, 18, 19, or 20 contiguous weeks. In some embodiments, the current dose of the biologic drug is administered to the subject at a current inter-dose interval for about 10 to 20, 11 to 19, 12, to 18, 12 to 17, 12 to 16, 13 to 17, 13 to 16, 13 to 15, 14 to 16, or 14 to 15 contiguous weeks. In some embodiments, the current dose of the biologic drug is administered to the subject at a current inter-dose interval at least once. In some embodiments, the current dose of the biologic drug is administered to the subject at a current inter-dose interval at least about once, twice, three time, four times, five times, six times, seven times, eight times, nine time, or ten times. In some embodiments, the current dose of the biologic drug is administered to the subject at a current inter-dose interval at most about once, twice, three time, four times, five times, six times, seven times, eight times, nine time, or ten times. In some embodiments, the current dose of the biologic drug is administered to the subject at a current inter-dose interval once, twice, three time, four times, five times, six times, seven times, eight times, nine time, or ten times.

In some embodiments, the pre-specified or predetermined threshold concentration of the biologic drug comprises between about 1 mg/L and 20 mg/L. In some embodiments, the pre-specified or predetermined threshold concentration of the biologic drug comprises between about 1 mg/L and 10 mg/L. In some embodiments, the pre-specified or predetermined threshold concentration of the biologic drug comprises between about 1 mg/L to 2 mg/L, 1 mg/L to 3 mg/L, 1 mg/L to 4 mg/L, 1 mg/L to 5 mg/L, 1 mg/L to 6 mg/L, 1 mg/L to 7 mg/L, 1 mg/L to 7.5 mg/L, 1 mg/L to 8 mg/L, 1 mg/L to 9 mg/L, 1 mg/L to 10 mg/L, 1 mg/L to 15 mg/L, 1 mg/L to 20 mg/L, 2 mg/L to 3 mg/L, 2 mg/L to 4 mg/L, 2 mg/L to 5 mg/L, 2 mg/L to 6 mg/L, 2 mg/L to 7 mg/L, 2 mg/L to 7.5 mg/L, 2 mg/L to 8 mg/L, 2 mg/L to 9 mg/L, 2 mg/L to 10 mg/L, 2 mg/L to 15 mg/L, 2 mg/L to about 20 mg/L, 3 mg/L to 4 mg/L, 3 mg/L to 5 mg/L, 3 mg/L to 6 mg/L, 3 mg/L to 7 mg/L, 3 mg/L to 7.5 mg/L, 3 mg/L to 8 mg/L, 3 mg/L to 9 mg/L, 3 mg/L to 10 mg/L, 3 mg/L to 15 mg/L, 3 mg/L to 30 mg/L, 4 mg/L to 5 mg/L, 4 mg/L to 6 mg/L, 4 mg/L to 7 mg/L, 4 mg/L to 7.5 mg/L, 4 mg/L to 8 mg/L, 4 mg/L to 9 mg/L, 4 mg/L to 10 mg/L, 4 mg/L to 15 mg/L, 4 mg/L to 20 mg/L, 5 mg/L to 6 mg/L, 5 mg/L to 7 mg/L, 5 mg/L to 7.5 mg/L, 5 mg/L to 8 mg/L, 5 mg/L to 9 mg/L, 5 mg/L to 10 mg/L, 5 mg/L to 15 mg/L, 5 mg/L to 20 mg/L, 6 mg/L to 7 mg/L, 6 mg/L to 7.5 mg/L, 6 mg/L to 8 mg/L, 6 mg/L to 9 mg/L, 6 mg/L to 10 mg/L, 6 mg/L to 15 mg/L, 6 mg/L to 20 mg/L, 7 mg/L to 7.5 mg/L, 7 mg/L to 8 mg/L, 7 mg/L to 9 mg/L, 7 mg/L to 10 mg/L, 7 mg/L to 15 mg/L, 7 mg/L to 20 mg/L, 7.5 mg/L to 8 mg/L, 7.5 mg/L to 9 mg/L, 7.5 mg/L to 10 mg/L, 7.5 mg/L to 15 mg/L, 7.5 mg/L to 20 mg/L, 8 mg/L to 9 mg/L, 8 mg/L to 10 mg/L, 8 mg/L to 15 mg/L, 8 mg/L to 20 mg/L, 9 mg/L to 10 mg/L, 9 mg/L to 15 mg/L, 9 mg/L to 20 mg/L, 10 mg/L to 15 mg/L, 10 mg/L to 20 mg/L, or 15 mg/L to 20 mg/L. In some embodiments, the pre-specified or predetermined threshold concentration of the biologic drug comprises between about 1 mg/L, 2 mg/L, 3 mg/L, 4 mg/L, 5 mg/L, 6 mg/L, 7 mg/L, 7.5 mg/L, 8 mg/L, 9 mg/L, 10 mg/L, 15 mg/L, or 20 mg/L. In some embodiments, the pre-specified or predetermined threshold concentration of the biologic drug comprises between about at least 1 mg/L, 2 mg/L, 3 mg/L, 4 mg/L, 5 mg/L, 6 mg/L, 7 mg/L, 7.5 mg/L, 8 mg/L, 9 mg/L, 10 mg/L, or 15 mg/L. In some embodiments, the pre-specified or predetermined threshold concentration of the biologic drug comprises between about at most 2 mg/L, 3 mg/L, 4 mg/L, 5 mg/L, 6 mg/L, 7 mg/L, 7.5 mg/L, 8 mg/L, 9 mg/L, 10 mg/L, 15 mg/L, or 20 mg/L.

In some embodiments, the subject has an immune mediated inflammatory disease. In some embodiments, the pre-specified or predetermined threshold concentration of the biologic drug comprises about 5 mg/L to about 10 mg/L when the biologic drug is ADA. In some embodiments, the pre-specified or predetermined threshold concentration of the biologic drug comprises about 5 mg/L to about 10 mg/L when the biologic drug is IFX. In some embodiments, the pre-specified or predetermined threshold concentration of the biologic drug comprises about 1 mg/L to about 7.5 mg/L when the biologic drug is TCZ.

In some embodiments, the model determines a likelihood of achieving a pre-specified threshold concentration of the biologic drug in the subject based, at least in part, on the level of the biologic drug, the level of the autoantibodies, and the level of albumin quantified. In some embodiments, the model determines a likelihood of achieving a pre-specified threshold concentration of the biologic drug in the subject based, at least in part, on the current dose of the biologic drug and the current inter-dose interval. In some embodiments, determining the likelihood of achieving the pre-specified threshold concentration of the biologic drug in the subject is further based, at least in part, on a weight of the subject. In some embodiments, determining the likelihood of achieving the pre-specified threshold concentration of the biologic drug in the subject comprises estimating a clearance rate of the biologic drug in the subject based, at least in part, on the weight of the subject and the level of albumin quantified. In some embodiments, determining the likelihood of achieving the pre-specified threshold concentration of the biologic drug in the subject further comprises determining whether the subject has a poor prognostic factor of pharmacokinetic origin (PPFPK). In some embodiments, the PPFPK is determined based, at least in part, on the level of the biologic drug quantified, the clearance rate, or both.

In some embodiments, a high likelihood may be a greater than 50%, 60%, 70%, 80%, 90%, 950%, 96%, 97%, 98%, or 99% chance or greater of achieving the pre-specified threshold concentration value. In some embodiments, a medium likelihood may be a greater than a 10% chance and up to a 50% chance of achieving the pre-specified threshold concentration value. In some embodiments, a low likelihood may be a 10% or less chance of achieving the pre-specified threshold concentration value.

In some embodiments, the model recommends a dosing regimen for a patient to achieve the pre-specified threshold concentration value. For example, if the model calculates a high likelihood of a patient achieving the pre-specified threshold concentration value, the model may recommend the patient stay on the current dosing regimen. In some embodiments, if the model calculates a medium to low likelihood of the patient maintaining the pre-specified threshold concentration value, the model may recommend the patient take a higher dose and/or shorten the dosing interval. For example, the model may recommend raising the dose from 40 mg to 50 mg and/or shortening the dosing interval from two weeks to one week. In some embodiments, if the model calculates that the patient will achieve a higher concentration value than the pre-specified threshold concentration value, the model may recommend the patient take a lower dose and/or increase the dosing interval. For example, the model may recommend lowering the dose from 40 mg to 30 mg and/or increasing the dosing interval from two weeks to three weeks. In some embodiments, the recommended dosing regimen is transmitted to a pharmacist or clinical decision tool. In some embodiments, the biological therapy is administered to the patient in accordance with the recommended dosing regimen.

In some embodiments, if the likelihood of achieving the pre-specified threshold concentration of the biologic drug is above 50%, then: (1) the current dose of the biologic drug is administered to the subject at the current inter-dose interval; or (2) a dose of the biologic drug is administered that is (i) lower than the current dose to the subject at the current inter-dose interval, (ii) the same as the current dose to the subject at an inter-dose interval that is longer than the current inter-dose interval, or (iii) lower than the current dose to the subject at an inter-dose interval that is longer than the current inter-dose interval. In some embodiments, if the likelihood of achieving the pre-specified threshold concentration of the biologic drug is below 50%, a dose of the biologic drug is administered that is (i) higher than the current dose to the subject in the current inter-dose interval, (ii) the current dose at an inter-dose interval that is shorter than the current inter-dose interval, or (iii) higher than the current dose to the subject in the inter-dose interval that is shorter than the current inter-dose interval.

In some embodiments, if the dose of the biologic drug is above a maximum dose and the inter-dose interval in is less than or equal to a minimum inter-dose interval, the treatment comprising the biologic drug is discontinued. In some embodiments, if the treatment comprising the biologic drug is discontinued, then the subject is administered with another biologic drug or small molecule that differs from the biologic drug. In some embodiments, the small molecule comprises a small molecule inhibitor. In some embodiments, the small molecule inhibitor is specific to a Janus Kinase (JAK). In some embodiments, the small molecule inhibitor specific to JAK comprises baricitinib, tofacitinib, or upadacitinib, or any combination thereof. In some embodiments, the small molecule is specific to a sphingosine 1-phosphate (S1P) modulator or an SIP receptor modulator. In some embodiments, the small molecule specific to an SIP receptor modulator comprises fingolimod, siponimod, ozanimod, or ponesimod, or any combination thereof.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a,” “an,” and “the” include plural references unless the context clearly dictates otherwise. Any reference to “or” herein is intended to encompass “and/or” unless otherwise stated.

Reference throughout this specification to “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

The term “biologic drug profile” generally refers to a profile of a subject disclosed herein. The biologic drug profile can comprise a dose of a biologic drug and an inter-dose interval estimated to achieve a threshold biologic drug concentration in a subject sufficient to treat the disease. The threshold biologic drug concentration may be a pre-specified threshold concentration.

Non-limiting examples of “biological sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term, “clinical disease activity index (CDAI)” refers to the patient global assessment of disease activity for an immune-mediated inflammatory disease disclosed herein, which may change according to industry standards. In some embodiments, the clinical disease activity index is the Crohn's disease activity index (CDAI), which is disclosed in Best et al., Predicting the Crohn's disease activity index from the Harvey-Bradshaw Index. Inflammatory Bowel Diseases 2006, 12 (4): 304-10, which is hereby incorporate by reference in its entirety. In some embodiments, the CDAI is a CDAI for rheumatoid arthritis as described in Aletaha D, Nell V P, Stamm T, et. al. Acute phase reactants add little to composite disease activity indices for rheumatoid arthritis: validation of a clinical activity score. Arthritis Research & Therapy 2005, 7 (4): R796-806, which is hereby incorporated by reference in its entirety.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “indel” as disclosed herein, refers to an insertion, or a deletion, of a nucleobase within a polynucleotide sequence.

In some embodiments, the terms “individual” or “subject” are used interchangeably and refer to any animal, including, but not limited to, humans, non-human primates, rodents, and domestic and game animals, which is to be the recipient of a particular treatment. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In various embodiments, a subject can be one who has been previously diagnosed with or identified as suffering from or having a condition in need of treatment. In certain embodiments, the subject is a human. In various other embodiments, the subject previously diagnosed with or identified as suffering from or having a condition may or may not have undergone treatment for a condition. In yet other embodiments, a subject can also be one who has not been previously diagnosed as having a condition (i.e., a subject who exhibits one or more risk factors for a condition). A “subject in need” of treatment for a particular condition can be a subject having that condition, diagnosed as having that condition, or at risk of developing that condition. In some embodiments, the subject is a “patient,” that has been diagnosed with a disease or condition described herein.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some instances, IBD comprises fibrosis, fibrostenosis, stricturing and/or penetrating disease, obstructive disease, or a disease that is refractory (e.g., mrUC, refractory CD), perianal CD, or other complicated forms of IBD.

The term “in vivo” is used to describe an event that takes place in a subject's body.

The term, “Linkage disequilibrium,” or “LD,” as used herein refers to the non-random association of alleles or indels in different gene loci in a given population. LD may be defined by a D′ value corresponding to the difference between an observed and expected allele or indel frequencies in the population (D=Pab-PaPb), which is scaled by the theoretical maximum value of D. LD may be defined by an r² value corresponding to the difference between an observed and expected unit of risk frequencies in the population (D=Pab-PaPb), which is scaled by the individual frequencies of the different loci. In some embodiments, D′ comprises at least 0.20. In some embodiments, r² comprises at least 0.70.

The term “model” generally refers to a computer simulation used herein to predict an output based on certain inputs. In some embodiments, the computer simulation may employ statistical methods, numerical methods, machine learning methods, or any combination thereof. In some embodiments, the output is a recommended dose or inter-dose interval for a biologic drug, a likelihood of clinical remission, or both. In some embodiments, the input comprises one or more analytes disclosed herein (e.g., CRP, IL-6, biologic drug, antibodies against the biologic drug, albumin), the body mass index (BMI) of the subject, the weight of the subject, information about the subject (e.g., disease severity, symptom severity and/or type, clinical remission status, age, gender, prior medial history, immune-compromising conditions, and the like).

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The term “poor prognostic factor of pharmacokinetic origin” or “PPFPK” generally refers to factors that can be used as predictors of achieving a predetermined output, such as for example, a pre-specified threshold concentration of the biologic drug or clinical remission of the disease in the subject. The PPFPK may comprise a level of the biologic drug quantified in a subject, an estimated clearance rate of the biologic drug in a subject, or both.

The term “pre-specified threshold” generally refers to a target concentration level of a drug. The term “pre-specified threshold” may be used interchangeably with “predetermined threshold” unless specified otherwise. A dose and an inter-dose interval of a drug administered to a subject may be adjusted based on a likelihood of achieving a pre-specified threshold of the drug in the patient as determined by a model disclosed herein.

The term “patient” or “subject” generally refers to an individual having a disease, such as, but not limited to, those disclosed herein. Subject specific data may be obtained from a patient or a subject, which may be used to establish a biologic drug profile for a patient or a subject.

The term “reference population” generally refers to a population of subjects. In some embodiments, the reference population is a population of subjects that have received a biologic drug for treatment of a disease or a condition disclosed herein. Generally, the subject is not part of the reference population. In some embodiments, the reference population comprises subjects that have received the same biologic drug as the subject. In some embodiments, the reference population may comprise subjects with the same disease as the subject. Data from the reference population may be used to develop and/or train a model of the present disclosure. In some embodiments, the model may be used to determine a treatment regimen for the subject, including, for example a dose or an inter-dose interval for a therapeutic agent disclosed herein.

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anti-12 antibody, and anti-Cbir1 flagellin antibody.

The term, “single nucleotide variant” or SNV as disclosed herein, refers to a variation in a single nucleotide within a polynucleotide sequence. The term should not be interpreted as placing a restriction on a frequency of the SNV in a given population.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

### EXAMPLES

The following examples are included for illustrative purposes only and are not intended to limit the scope of the invention.

### Example 1: Adalimumab Dosing Optimization

An in silico prediction of dosage amounts and inter-dose intervals (e.g., time between doses) was performed based on pharmacokinetic model disclosed herein, which is referred to herein as “clinical decision tool.” The clinical decision tool described herein, in some embodiments, comprises is a computational model engineered to estimate dosage amounts and inter-dose intervals of biologic drugs for the treatment of immune-mediated inflammatory disease (e.g., inflammatory bowel disease, rheumatoid arthritis, and the like) in subjects having the immediate-mediated inflammatory disease. In this example, the subject has an inflammatory bowel disease (IBD) and the biologic drug is Adalimumab.

### Obtaining a Sample

Individual serum samples from healthy controls were obtained from blood bank donors (Golden West Biologics, Temecula, CA). Sera from patients with rheumatoid arthritis (RA), PS and inflammatory bowel disease (IBD) treated with adalimumab (ADA) every 14 days were drawn according to a protocol approved by an Institutional Review Board/Ethics Committee. Unless otherwise noted, all reagents and chemicals were obtained from Thermo Fisher Scientific (Waltham, MA) or Sigma Aldrich Corporation (St. Louis, MO). ADA and anti-ADA antibody levels were determined using homogenous mobility shift assay.

### Mobility Shift Assay

A mobility shift assay was performed on the biological sample obtained from the subject.

ATA calibration serum. Antibodies against ADA (ATA)-positive sera were prepared by immunizing two rabbits with purified adalimumab (ProSci, Inc., San Diego, CA). Bleeds of anti-adalimumab positive sera from the rabbits were pooled and the relative amount of ATA was arbitrarily defined as 100 U/mL, equal to 1:100 dilutions. The pooled ATA calibration serum was aliquoted and stored at −70° C.

Conjugation of adalimumab and TNF-α. The method for the conjugation of AlexaFluor-488 to adalimumab was same as described previously. Briefly, commercially available adalimumab (Humira®, Abbott Laboratories, Abbott Park, IL) was buffer exchanged with phosphate buffered saline (PBS, pH 7.3) and labeled with AlexaFluor-488 (Life Technology, Carlsbad, CA) following the manufacturer's instructions. Only those conjugates containing 2-3 fluorescent dyes per antibody qualified for the ATA-HMSA. Conjugation of AlexaFluor-488 to TNF-α was performed as described previously.

HMSA for ATA and adalimumab. The procedure for the ATA-HMSA and the adalimumab-HMSA were similar to the ATI-HMSA as described previously, except that AlexaFluor-488 labeled adalimumab was used in the ATA-HMSA. In brief, serum samples were first acid dissociated with 0.5 M citric acid (pH 3.0) for 1 h at RT, and then neutralized with 10×PBS (pH 7.3) in the presence of adalimumab-AlexaFluor-488 in a 96-well plate format. The plate was incubated for 1 h at RT on an orbital shaker to complete the formation of the immune complexes. The equilibrated samples were filtered through a MultiScreen-Mesh Filter plate equipped with a Durapore membrane (0.22 μm; EMD Millipore, Billerica, MA) into a 96-well receiver plate (Nunc, Thermo Fisher Scientific, Waltham, MA). The recovered solutions were individually loaded into an HPLC system (Agilent Technologies 1200 series HPLC system, Santa Clara, CA) equipped with a BioSep SEC-3000 column (Phenomenex, Torrance, CA). The chromatography was run at the flow-rate of 1 mL/min with 1×PBS (pH 7.3) as the mobile phase for a total of 20 min, and was monitored with a fluorescence detector at excitation and emission wavelengths of 494 nm and 519 nm, respectively. ChemStation Software (Agilent Technologies, Santa Clara, CA) was used to set-up and collect data from the runs automatically and continuously.

To generate a standard curve, one aliquot of the stock ATA calibration serum was thawed and diluted to 2% in volume with rabbit serum (Sigma Aldrich, St. Louis, MO) in HPLC assay buffer (1×PBS, pH 7.3) to achieve final concentrations in the assay wells of 0.031, 0.063, 0.125, 0.250, 0.500, 1.000, 2.000, and 4.000 U/mL. Three quality control (QC) samples were prepared by diluting the calibration serum in assay buffer with 0.1% BSA to yield the high (1.600 U/mL), mid (0.600 U/mL), and low (0.200 U/mL) control concentrations. Similarly, adalimumab calibration standards were prepared by serially diluting purified adalimumab with assay buffer containing 0.1% BSA to achieve final concentrations of 0.013, 0.025, 0.050, 0.100, 0.200, 0.400, 0.800 and 1.600 μg/mL of adalimumab and final NHS concentration of 4% in the reaction mixture. Three adalimumab QC samples were prepared by diluting the adalimumab calibration standard with assay buffer and 0.1% BSA to yield the high (25 μg/mL), mid (10 μg/mL), and low (5 μg/mL) control concentrations.

ATA-HMSA and adalimumab-HMSA evaluation. The analytical validations including the performance characteristics for the ATA-HMSA and the adalimumab-HMSA (calibration standards, assay limits, assay precision [intra- and inter-assay], linearity of dilution, and substance interference) were performed based on the industrial recommendations. A panel of serum samples from drug-naïve healthy donors (n=100; Golden West Biologics. Temecula, CA) were analyzed to determine the assay cut points for the ATA-HMSA and adalimumab-HMSA. The samples were normally distributed and parametric statistics were applied to determine the cut point. The assay cut points were defined as the threshold above which samples were deemed to be positive, and was set to have an upper negative limit of approximately 99%, calculated by using the lowest mean value of individual samples interpolated from the standard curve+3.0× the standard deviation (SD).

2.5. Data analysis. Data analysis was performed with the use of a proprietary automated program run on R software (R Development Core Team, Vienna, Austria). Briefly, the R program opened the ChemStation files, normalized the spectra, determined the area under each peak, and calculated the proportion of total peak areas shifted to the bound ATA/adalimumab-AlexaFluor-488 complexes over the total bound and free adalimumab-AlexaFluor-488 peak areas in the ATA-HMSA. An exponential association standard curve was generated from the standards and the measured ATA values were interpolated from the curve. To obtain the actual ATA and adalimumab concentrations in the serum, the interpolated results from the standard curves were multiplied by the dilution factor.

### Solid Phase Assay

Although a mobility shift assay was performed on the biological sample in this example, it is also possible to perform a solid phase assay, such as an enzyme mobility shift assay (ELISA), liquid chromatography coupled with mass spectrometry, reporter gene assay, or chemiluminescent assay. An ELISA may be performed according to the protocols disclosed in Bendtzen K. Is There a Need for Immunopharmacologic Guidance of Anti-Tumor Necrosis Factor Therapies. ARTHRITIS & RHEUMATISM Vol. 63, No. 4, April 2011, pp 867-870, or Jaminitski A et al. The presence or absence of antibodies to infliximab or adalimumab determines the outcome of switching to etanercept. RHEUM DIS. 2011 February; 70(2):284, each of which is hereby incorporated by reference in its entirety. A reporter gene assay can also be performed on the biological sample with a protocol disclosed in Lallemand, et a., Reporting gene assay for the quantification of the activity and neutralizing antibody response to TNFα antagonists. J. IMMUN. METHODS. 373 (2011) 229-239, which is hereby incorporated by reference in its entirety.

The levels of ADA and ATA levels detected using the mobility shift assay in the sample were input into a clinical decision tool described herein.

### Patient Information

Patient information, such as the subject's weight, disease severity (e.g., remission, recurrence, or type, frequency, and/or severity of symptoms well-being and patient reported outcome forming standard SF-36 or EQ5D among others disclosed herein is collected from the subject remotely. The remote collection of this information is obtained using a mobile application on a personal electronic device of the subject, such as a smartphone, tablet, or wearable electronic device. Non-limiting examples of the wearable electronic device include a fitness tracker (e.g., watch, wristband, ring, chest monitor), glucose monitor, heart rate monitor, sleep tracking device, and the like. The patient information may be self-reported by the subject, such as through manual entry into a mobile application on the subject's smartphone. The patient information may be automatically saved to a data store, such a cloud-based data store and retrieved from the data store by the clinical decision tool.

### Estimating Individual Pharmacokinetic Parameters Using the Clinical Decision Tool

Individual pharmacokinetic parameters were estimated using a combination of non-linear mixed effect models (NONMEM). The model employs 1s′ order absorption with one compartment and linear elimination with random effects on clearance (Cl), volume of distribution (central, [V1 Covariates consists of patient weight (on Cl, V1), albumin (ALB) on Cl, and c-reactive protein (CRP) on Cl. F or the subject, ADA levels and weight were used to estimate the conditional distribution of the individual pharmacokinetic parameters. The conditional distribution represents the uncertainty of the individual parameters given the observations collected, and the prior information. Conditional distributions of the individual pharmacokinetic parameters (Cl, V1) were generated for the subject using Markov Chain Monte Carlo (MCMC) simulations (Metropolis Hastings algorithm), and sampling (n=100) from those distributions were used to estimate estimated trough levels and likelihood to achieve exposure (e.g., biologic drug concentration) above 5 to 10 micrograms per milliliter (μg/mL). Prediction intervals (80% corresponding to the 10^{th} and 90^{th} percentile of the estimated levels) were also calculated.

Next, 100 sets of pharmacokinetic parameters were sampled from the subject's conditional distribution and used to estimate the end of cycle concentration, median trough concentration (immediately before injection, 14 days post infusion) and probability of achieving a trough concentration above 5 and 10 μg/ml. The median and 80% prediction interval (corresponding to the 10th and 90th percentile of the estimates) were reported.

The output of the clinical decision tool described herein was the probability (range 0-100%) of achieving the pre-specified threshold of ADA (e.g., >7.5 mg/L), the achievement of a high likelihood of achieving the pre-specified threshold of ADA (e.g., >90%), and a dosing recommendation were determined. Next, the clinical decision tool calculated inter-dose intervals (timing between doses) that were predicted to sustain the pre-specified threshold of ADA of above 7.5 mg/L in the subject with a high likelihood (>90%).

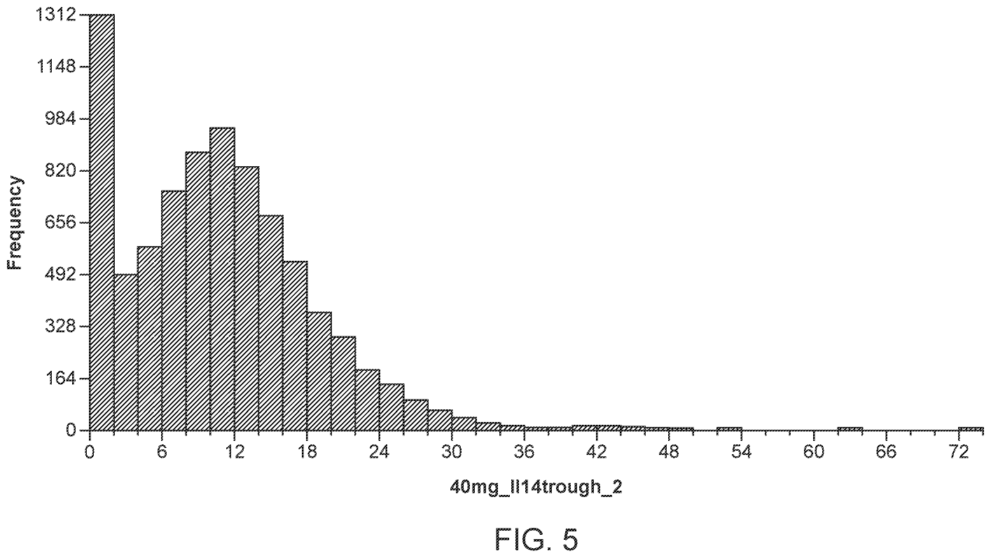

FIG. 5 is a graph showing a distribution of adalimumab levels within a typical IBD patient population as calculated using the clinical decision tool by sampling from the conditional distribution of the parameter estimates and applying one compartment equation to calculate the trough. A “typical” IBD patient population as used herein refers to IBD patients having received ADA for treatment of the IBD. Table 3 provides ADA levels in a typical IBD patient population by dose and dose interval as predicted using the clinical decision tool.

TABLE 1
ADA Levels in a Typical IBD Patient Population

| | Weekly | Two weeks | | Three weeks | |
|---|---|---|---|---|---|
| 20 mg | 10.9 mg/L | 5.3 | mg/L | 3.5 | mg/L |
| 40 mg | 21.7 mg/L | 10.7 | mg/L | 7.0 | mg/L |
| 80 mg | 43.4 mg/L | 21.4 | mg/L | 14.0 | mg/L |

Table 4 provides the probabilities of achieving the threshold concentration of above 7.5 mg/L of ADA in this typical IBD patient population.

TABLE 2
Probabilities of Achieving a Threshold Concentration
Value in a Typical IBD Patient Population

| | Weekly | Two weeks | Three weeks | Four weeks |
|---|---|---|---|---|
| 20 mg/0.4 ml pen | 51% | 13% | 2% | <1% |
| 40 mg/0.4 ml pen | 74% | 50% | 27% | 5% |
| 80 mg/0.8 ml pen | 82% | 73% | 61% | 52% |

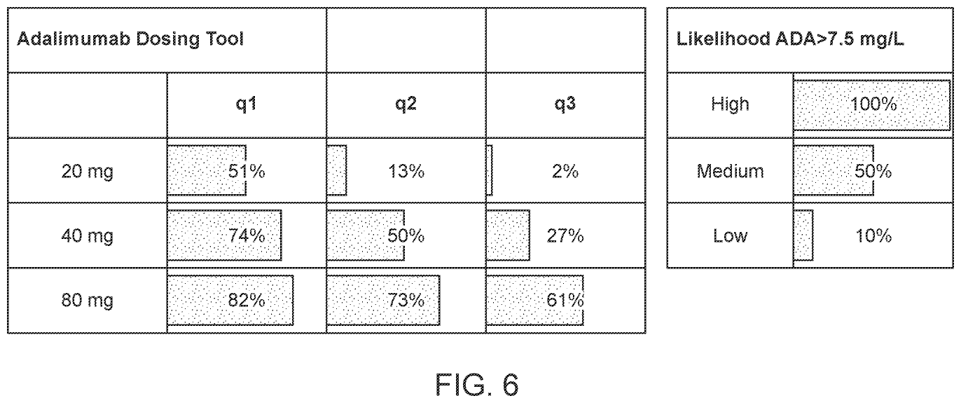

Referring to Table 4, a typical IBD patient is receiving the standard ADA dose of 40 mg every other week. The clinical dosing tool predicts, as further illustrated in FIG. 6, that this patient has a medium likelihood (50%) to achieve an ADA concentration above 7.5 mg/L. Shortening the inter-dose interval to weekly schedule increases the likelihood to 74%. Without being bound by any particular theory, the output of the clinical decision tool as described herein may be helpful to guide clinically relevant decision-making by medical professionals. FIG. 6 provides the percent likelihood of maintaining a concentration of ADA that is over 7.5 mg/L in a typical IBD patient receiving the dosage amounts shown (20 mg, 40 mg, 80 mg) at a frequency of every week (“q1”), every other week (“q2”), and every three weeks (“q3”).

### Prophetic Applications of in Silico Modeling Using the Clinical Decision Tool

As an example, the output may be transmitted to a medical professional, like a physician, to recommend to the medical professional a treatment regimen (e.g., dose amount and inter-dose interval).

For example, if the goal is to maintain the pre-determined threshold of 5 mg/L of ADA, the clinical decision tool may recommend that the physical prescribe the current dosing regimen (40 mg every two weeks) in the presence of a high probability of achieving a pre-specified threshold of 5 mg/L of over 90%. By contrast, the clinical decision tool may recommend the shortening of the inter-dose interval from every two weeks to every week in order to achieve a pre-specified threshold (e.g. >90% at 7.5 mg/L), or the prolongation of the inter-dose interval to every 3 weeks, or 4 weeks if the proposed inter-dose interval maintains the probability of ADA above 90% at the pre-specified threshold. Other alternatives consist of 20 mg or 80 mg dosing as necessary.

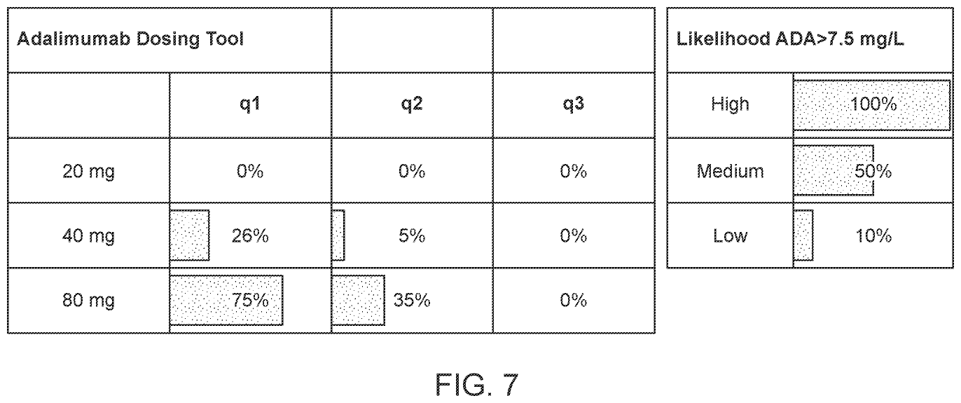

In another example, as shown in FIG. 7, a subject with IBD that is under-exposed to ADA (has less than the pre-determined threshold amount of ADA) when receiving a standard ADA dose of 40 mg every other week may have a low likelihood (<50%) to achieve an ADA concentration above 7.5 mg/L at the current dose. In this example, the clinical decision tool may recommend that the dose interval be shortened to weekly dosing and/or that the dose be increased to 80 mg.

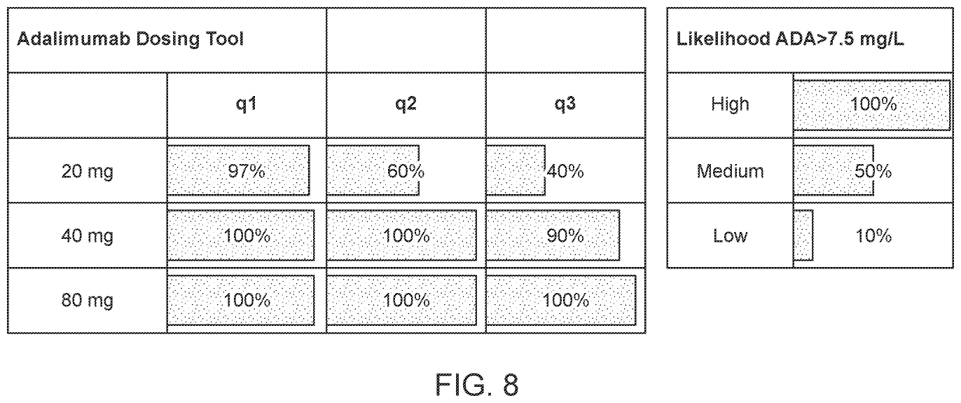

By contrast, as shown in FIG. 8, a subject overexposed to ADA when receiving the standard ADA dose of 40 mg every other week may have a high likelihood to achieve an ADA concentration above 7.5 mg/L at the current dose. The clinical decision tool may estimate the probability and confidence to be above the pre-specified threshold with a lower dose than 40 mg (and same inter-dose interval), or the same dose administered with elongated inter-dose intervals that are less frequent than every other week. Treatment decision is to prolongate the dose interval to every three weeks.

Table 5 illustrates the individual parameter estimates with covariates used in the Clinical Decision Tool described in some embodiments herein.

TABLE 3
Individual Parameter Estimates with Covariates for Adalimumab (ADA)

| | | | | Add3 | Add4 + |
|---|---|---|---|---|---|
| | | Add 1 | Add2 | ATA + | ATA + ALB + |
| Project Name | base | ATA | ATA + ALB | ALB + CRP | CRP + WT |
| −2*LL | 3861.23 | 3765.8 | 3705.48 | 3686.29 | 3677.89 |
| (Importance sampling) | | −95.43 | −155.74 | −174.94 | −183.34 |
| ka_pop | 0.2 | 0.2 | 0.2 | 0.2 | 0.2 |
| V_pop | 10 | 11.53 | 10.21 | 10.8 | 10.7 |
| | 29.58% | 9.05% | 13.41% | 8.1% | 7.3% |
| Cl_pop | 0.32 | 0.26 | 0.26 | 0.25 | 0.25 |
| | 5.49% | 5.55% | 5.21% | 4.7% | 14.72% |
| beta_Cl_ATA_CAT_1 | | 1.21 | 1.11 | 1.06 | 1.01 |
| | | 9.15% | 9.88% | 7.23% | 9.33% |
| beta_Cl_logtAlbumin | | | −1.27 | −0.75 | −0.61 |
| | | | 21.96% | 32.56% | 38.92% |
| beta_Cl_CRP | | | | 0.012 | 0.014 |
| | | | | 12.37% | 13.78% |
| beta_Cl_logtWeight | | | | | 0.54 |
| | | | | | 53.7% |
| omega_V | 0.33 | 0.53 | 0.38 | 0.38 | 0.3 |
| | 113.49% | 24.49% | 44.96% | 22.21% | 32.47% |
| omega_Cl | 0.59 | 0.57 | 0.52 | 0.51 | 0.49 |
| | 8.61% | 7.72% | 7.71% | 7.29% | 7.84% |
| b | 0.36 | 0.31 | 0.31 | 0.31 | 0.32 |
| | 4.49% | 4.29% | 5.53% | 4.05% | 4.09% |

ADA = adalimumab
ATA = antibodies against ADA
ALB = albumin
CRP = c-reactive protein
V = volume
Ka = rate at which ADA enters the system (Kₐ)
CL = clearance
beta_Cl_ATA_CAT_1 = estimate associated with Antibody to Adalimumab status on clearance
beta_Cl_logtAlbumin = estimate associated with Albumin on clearance
beta_Cl_CRP = estimate associated with CRP on clearance
beta_Cl_logtWeight = estimate associated with weight on clearance
Omega_V = CV around the reference value for V.
Omega_CL = CV around the reference value for CL.

### Example 2: Infliximab Dosing Optimization Using Clinical Decision Tool

Another in silico prediction of dosage amounts and inter-dose intervals (e.g., time between doses) was performed using the clinical decision tool described herein. The clinical decision tool described herein, in some embodiments, comprises is a computational model engineered to estimate dosage amounts and inter-dose intervals of biologic drugs for the treatment of immune-mediated inflammatory disease (e.g., inflammatory bowel disease, rheumatoid arthritis, and the like) in subjects having the immediate-mediated inflammatory disease. In this example, the subject has an inflammatory bowel disease (IBD) and the biologic drug is Infliximab (IFX).

A sample is obtained from a subject during the elimination phase following IFX after 21 days after the last dose. IFX and anti-IFX antibody levels were measured in the sample using the mobility shift assay described in Example 1. Patient information is obtained from the subject, including the weight, disease severity (e.g., remission, recurrence), or type, frequency, and/or severity of symptoms of the subject remotely, such by a mobile application on a personal electronic device.

Albumin was measured in the sample by the HMSA assay disclosed in Example 1. Albumin is recirculated by the neonatal receptor and is a marker of recycling of the monoclonal antibody against IFX The higher the albumin the lower the clearance and the higher the exposure of IFX. C-Reactive Protein (CRP) was measured in the sample by the HMSA assay disclosed in Example 1.

### Determining the Estimated Target Dose and Inter-Dose Interval

IFX levels, anti-IFX antibody levels, albumin levels, and CRP levels and weight imputed in the clinical decision tool described in Example 1. The output consisted of the probability (range 0-100%) of achieving a pre-specified Threshold of IFX at the beginning of the next infusion cycle (>5 mg/L, 7.5 mg/L, 10 mg/L), the achievement of a high likelihood of achieving the pre-specified threshold of IFX at the beginning of the next infusion cycle (>50%, >75%, >90%) and, the estimate for a target dose and inter-dose interval (4 to 10 weeks) to maintain or achieve the high likelihood of IFX levels (>90%).

### Clinical Decision

Based on the estimated target dose and inter-dose interval determined above, the following dosing recommendation is transmitted to a pharmacist and clinical decision tool. Anon-limiting example of the recommendation is provided in FIG. 8. The decision is based on the maintenance of the current dosing regimen (5 mg/Kg every 8 weeks) in the presence of a high probability of achieving pre-specified threshold (e.g. >90% at 5 mg/L). The dose can increase (5 to 7.5 or 10 mg/Kg) or the inter-dose interval can shorten from every 8 weeks to every 7 weeks, 6 weeks, 5 weeks and 4 weeks to achieve the pre-specified threshold (e.g. >90% at 5 mg/L). The recommendation can also be to switch therapy to adalimumab or another targeted monoclonal antibody in the event the proposed dose is above 10 mg/Kg. A lower dose (10 to 7.5 to 5 mg/Kg) or the prolongation of the inter-dose interval to every 9 week, 10 weeks or 12 weeks can be recommended if the proposed inter-dose interval maintains the probability of IFX above 90% at the pre-specified Threshold.

### Example 3. Infliximab Dosing Optimization for a Subject with Crohn's Disease

The clinical decision tool described herein utilizes a Bayesian data assimilation, machine learning algorithm to combine patient weight, current dose and interval, measured serum IFX, measured anti-IFX antibodies and albumin levels to predict optimal, individualized IFX dosing. It arms you with the evidence to validate your treatment plans and investigate alternative doses and intervals.

In this example, a subject that is male, 43 years old with Crohn's disease is undergoing IFX maintenance therapy, receiving 500 mg every 8 weeks (6 mg/Kg). A sample is collected using methods and devices of previous examples 21 days after the infusion (mid cycle) and sent to the clinical laboratory. Weight (83 Kg) and disease activity indicating the absence of symptoms are transmitted to the clinical decision tool through a mobile application on the subject's smartphone or tablet. The following are measured in the sample: IFX level (17 mg/L), ATI status (<3.1 U/ml), Albumin (43.6 g/L) and CRP (5 mg/L). These measurements are input into the clinical decision tool described above, which utilizes Bayesian data assimilation. Table 6 provided the individual parameter estimates for this subject.

TABLE 4
Individual Parameter Estimates

| | Estimate for the patient output |
|---|---|
| Parameter | of conditional distribution |
| Pharmacokinetic parameter | Clearance = 0.392 ± 0.025 L/day |
| estimates | V1 = 4.25 ± 0.36 L |
| | Q = 0.103 ± 0.006 L/day |
| | V2 = 1.364 ± 0.78 |
| Estimated Trough pre-infusion | 2.2 mg/L (80% PI: 1.13-3.38); Subtherapeutic |
| Probability of Trough > 3 mg/L | 21.2% (below 90%) |
| Probability of Trough > 5 mg/L | 6.3%% (below 90%) |
| Probability of Trough > 10 mg/L | 0%% (below 90%) |

Table 7 shows estimated trough concentrations calculated by the clinical decision tool for the patient using different dosing and dosing interval options.

TABLE 5
Estimated Trough Concentrations

| | | 4 weeks | 6 weeks | 8 weeks | 10 weeks |
|---|---|---|---|---|---|
| 5 | mg/Kg | 10.8(8.0-13.6) mg/L | 2.4(4.2-5.8) mg/L | 2.2 (1.1-3.4) mg/L | <1 mg/L |
| 7.5 | mg/Kg | 16.1(12.4-20.6) mg/L | 6.3 (3.8-9.0) mg/L | 2.7 (1.4-4.5) mg/L | <1 mg/L |
| 10 | mg/Kg | 21.0(15.7-28.1) mg/L | 8.4 (5.6-11.2) mg/L | 3.7 (1.8-5.7) mg/L | <1 mg/L |

Table 8 illustrates the probabilities of achieving a pre-specified trough concentration of above 5 mg/L in the patient using the different dosing and dosing interval options.

TABLE 6
Estimated Probabilities of Achieving Pre-Specified Trough Concentration

| | | 4 weeks | 6 weeks | 8 weeks | 10 weeks |
|---|---|---|---|---|---|
| 5 | mg/Kg | >3 mg/L: 100% | >3 mg/L: 77% | >3 mg/L: 14% | >3 mg/L: 0% |
| | | >5 mg/L: 100% | >5 mg/L: 25% | >5 mg/L: 4% | >5 mg/L: 0% |
| | | >10 mg/L: 47% | >10 mg/L: 3% | >10 mg/L: 0% | >10 mg/L: 0% |
| 7.5 | mg/Kg | >3 mg/L: 100% | >3 mg/L: 96% | >3 mg/L: 31% | >3 mg/L: 15% |
| | | >5 mg/L: 100% | >5 mg/L: 54% | >5 mg/L: 13% | >5 mg/L: 4% |
| | | >10 mg/L: 97% | >10 mg/L: 12% | >10 mg/L: 2% | >10 mg/L: 0% |
| 10 | mg/Kg | >3 mg/L: 100% | >3 mg/L: 100% | >3 mg/L: 41% | >3 mg/L: 15% |
| | | >5 mg/L: 100% | >5 mg/L: 95% | >5 mg/L: 20% | >5 mg/L: 5% |
| | | >10 mg/L: 99% | >10 mg/L: 24% | >10 mg/L: 4% | >10 mg/L: 0% |

Based on the data shown in Table 8, the clinical decision tool then estimates a recommended dose for each of the dosing and dosing interval option to achieve the pre-specified threshold trough concentration. This information is shown in Table 9. For example, the clinical decision tool estimates a 1.9 mg/Kg dose of IFX administered every 4 weeks to the subject will achieve a pre-specified threshold trough concentration of IFX of 3 mg/Lin the subject.

TABLE 7
Estimated Doses

| 4 weeks | 6 weeks | 8 weeks | 10 weeks |
|---|---|---|---|
| >3 mg/L: 1.9 mg/Kg | >3 mg/L: 6.4 mg/Kg | >3 mg/L: above | >3 mg/L: above |
| | | 10 mg/Kg | 10 mg/Kg |
| >5 mg/L: 3.2 mg/Kg | >5 mg/L: above | >5 mg/L: above | >5 mg/L: above |
| | 10 mg/Kg | 10 mg/Kg | 10 mg/Kg |
| >10 mg/L: 6.3 mg/Kg | >10 mg/L: above | >10 mg/L: above | >10 mg/L: above |
| | 10 mg/Kg | 10 mg/Kg | 10 mg/Kg |

Table 10 shows the parameter estimates used in each of the dosing and dosing interval options outlined above.

TABLE 8
Parameter Estimates

| Parameter | Model 1 | Model 2 | Model 3 |
|---|---|---|---|
| CL_pop (L/day, 70 Kg person) | 0.277 | 0.352 | 0.294 |
| V1_pop (L, 70 Kg person) | 4.90 | 3.406 | 3.33 |
| Q_pop (L/day, 70 Kg person) | 0.0201 | 0.147 | 0.0719 |
| V2_pop (L per 70 Kg person) | 0.844 | 1.274 | 1.14 |
| Omega CL | 0.285 | 0.307 | 0.327 |
| Omega V1 | NA | 0.126 | 0.15 |
| Omega Q | NA | NA | 1.10 |
| Omega V2 | NA | 0.533 | 0.799 |
| beta_Cl_ATI_1 | 0.562 | 0.255 | 0.229 |
| beta_CL_IMM | NA | —0.147 | NA |
| beta_Cl_logtALB | —1.13 | —0.855 | —1.17 |
| beta_Cl_logtWEIGHT | NA | 0.687 | 0.614 |
| beta_V1_logtWEIGHT | NA | 0.767 | 0.691 |
| beta_Q_logtWEIGHT | NA | NA | 1.1 |
| beta_V2_logtWEIGHT | NA | 0.412 | 0.59 |
| Additive error | 0.695 | 0.371 | NA |
| Proportional error | NA | 0.1 | 0.1 |

### Example 4. Pharmacokinetic Value Based Pricing for Tocilizumab

Another in silico prediction of dosage amounts and inter-dose intervals (e.g., time between doses) was performed using the clinical decision tool described herein. The clinical decision tool described herein, in some embodiments, comprises is a computational model engineered to estimate dosage amounts and inter-dose intervals of biologic drugs for the treatment of immune-mediated inflammatory disease (e.g., inflammatory bowel disease, rheumatoid arthritis, and the like) in subjects having the immediate-mediated inflammatory disease. In this example, the subject has an rheumatoid arthritis and the biologic drug is Tocilizumab (TCZ).

A sample is obtained from a subject with rheumatoid arthritis during treatment with TCZ. In this example the sample is a blood serum sample obtained by phlebotomy. However, other methods are envisioned, such as using a micro sampling device. Albumin, c-reactive protein (CRP), interleukin 6 (IL-6), TCZ and anti-TCZ antibody levels were measured in the sample using the mobility shift assay described in Example 1. Albumin is recirculated by the neonatal receptor and is a marker of recycling of the TCZ. The higher the albumin the lower the clearance and the higher the exposure to TCZ. Patient information is obtained from the subject, including the weight, disease severity (e.g., remission, recurrence), or type, frequency, and/or severity of symptoms of the subject remotely, such by a mobile application on a personal electronic device. Prior information from the reference population of RA patients having been treated with TCZ is obtained, including TCZ levels, anti-TCZ antibody levels, IL-6, C-Reactive Protein (CRP), weight of the patient, clinical remission status, TCZ level after switching to a new dosing regimen, and albumin.

Individual pharmacokinetic parameters are estimated using a combination of non-linear mixed effect models (NONMEM) and prior information from an hybrid of two previously reported pharmacokinetic (PK) model with modifications to add a 10% proportional error model constants. The two previously reported PK models are provided in Abdallah H, et al., Pharmacokinetic and Pharmacodynamic Analysis of Subcutaneous Tocilizumab in Patients With Rheumatoid Arthritis From 2 Randomized, Controlled Trials: SUMMACTA and BREVACTA. J CLIN PHARMACOL. 2017 April; 57(4):459-468; Frey N. et al., Population pharmacokinetic analysis of tocilizumab inpatients with rheumatoid arthritis. J CLIN PHARMACOL. 2010 July; 50(7):754-66, each of which is hereby incorporated by reference in its entirety. The model employs 1^{st} order absorption with two compartment and linear nonlinear Michaelis Menten with random effects on clearance (Cl), volume of distribution (central, [V1] and peripheral [V2]) and Vmax. In this example, covariates comprised the patient's weight (on Cl, V1). For each subject, TCZ levels, weight are used to estimate the conditional distribution of the individual parameters, which represents the uncertainty of the individual parameters given the observations collected, and the prior information. Conditional distributions of the model parameters (Cl, V1, V2 and Vmax) are generated for each patient using Markov Chain Monte Carlo (MCMC) simulations (Metropolis Hastings algorithm), and sampling (n=100) from those distributions are used to estimate estimated trough levels and likelihood to achieve exposure above 5 μg/mL as an example. Prediction intervals (80% corresponding to the 10^{th} and 90^{th} percentile of the estimated levels) are also calculated.

For each specimen, the conditional distribution represents the uncertainty of the individual parameter values. The conditional distribution estimation task permits to sample from this distribution to calculate the conditional mean and standard deviation which represents the uncertainty of the individual's parameter value, taking into account the observed TCZ level at the time of the specimen collection and the covariate values. The algorithm to estimate the conditional distribution of the model parameters employs a MCMC procedure, specifically Metropolis-Hastings algorithm. The algorithm works iteratively: at each iteration, a new individual parameter value is drawn from a proposal distribution. The new value is accepted with a probability after computation of the individual parameters for that iteration. Next, 100 sets of pharmacokinetic parameters were sampled from each individual's conditional distribution and used to estimate the end of cycle concentration, median trough concentration (immediately before infusion, 56 days post infusion) and probability of achieving a trough concentration above 5 and 10 μg/ml. The median and 80% prediction interval (corresponding to the 10th and 90th percentile of the estimates) were reported.

The conditional distribution of the individual distribution of the parameter was estimated using the clinical decision tool. This is the structural model to be applied to the clinical decision tool to calculate the poor prognostic factor of pharmacokinetic origin. The model can be a trained using the prior information comprising the TCZ levels, antibodies to TCZ, albumin, weight of the patient, clinical remission status, and the TCZ level after switching to anew dosing regimen.

The exposure at trough and probability to have a concentration above 10 mg/L or 5 mg/L or 10 mg/L are derived and the probability for that threshold to occur is estimated using the clinical decision tool. For each patient the probability to present with exposure commensurate with response is used. In this example we have 100% change to achieve concentration.

A recommendation to adjust the dosing of TCZ to maintain concentration above 5 mg/L with 90% confidence is provided by the clinical decision tool to a medical professional.

The active or inactive disease (e.g., RA) status of the patient is integrated into the clinical decision tool. In this example, the inter-dose interval is elongated in the presence of inactive disease when in the absence of the poor prognostic factor of pharmacokinetic origin; and shortened in the presence of active disease status and presence of poor prognostic factor of pharmacokinetic origin. In this example, the covariates are provided in Table 11.

Pharmacokinetic value based pricing is determined based on the calculated probability that exposure of TCZ (of the pre-determined threshold) is achieved in the patient with high confidence. In this example, the pharmacokinetic value based pricing is provided to the pharmacy benefit manager.

The prescription is initiated by the clinician with the recommendation of the pharmacist.

FIG. 9A and FIG. 9B illustrate non-limiting examples of workflows using the clinical decision tool. Referring to FIG. 9A, provided is an exemplary workflow for determining treatment for a subject with rheumatoid arthritis according to some embodiments herein. As shown, a patient may receive an initial dosing of a biologic drug, such as TCZ, based on initial patient information, such as the weight of the patient. For example, since a patient's weight is less than 100 kg, the subject receives an initial dosing every other week (“q2wk”) for 10 weeks. A specimen, such as the blood, may then be collected from a patient and it is evaluated whether the subject has a given threshold drug concentration with a certain likelihood. In this case, it is evaluated whether the subject has TCZ>5 mg/L with 90% confidence. If so, the subject's inter-dose interval for treatment can be elongated to every three weeks “q3wk”. In some embodiments, a model may predict whether at an inter-dose interval of every three weeks, the subject will continue to achieve a threshold biological drug concentration (e.g., TCZ>5 mg/L) with a certain likelihood (e.g., 90%). The model may make a prediction based on subject specific data, such as those described herein. If the output is yes, a dosing of every three weeks is initiated. If the output is no, a dosing of every other week is continued. If the subject has not achieved TCZ>5 mg/L with 90% confidence, the subject's inter-dose interval for treatment can be shortened to every week “q1wk”. In some embodiments, a model may predict whether at an inter-dose interval of every week, the subject will continue to achieve a threshold biological drug concentration (e.g., TCZ>5 mg/L) with a certain likelihood (e.g., 90%). If the output is yes, a dosing of every week is initiated. If the output is no, the model my further evaluate whether the inter-dose interval should be further shortened. For example, the model may evaluate whether an inter-dose interval of twice every week (“2q1w”) would achieve TCZ>5 mg/L with 90% confidence in a subject.

FIG. 9B further provides an exemplary workflow for determining treatment for a subject with rheumatoid arthritis according to some embodiments herein. As shown, a patient may receive a biologic drug every week for ten weeks as an initial dosing based on their weight of greater than 100 kg. After the ten weeks, a specimen, such as blood, is collected and it is evaluated whether the subject has a given threshold drug concentration with a certain likelihood. In this case, again, it is evaluated whether the subject has TCZ>5 mg/L with 90% confidence. If not, it is evaluated whether the subject's inter-dose interval for treatment can be shortened to twice every week. A model may predict whether at an inter-dose interval of twice every week, a subject can achieve TCZ>5 mg/L with 90% confidence. If so, a subject initiates a dosing of twice every week. If the model predicts that a subject still cannot achieve TCZ>5 mg/L with 90% confidence with an inter-dose interval of twice every week, the weight loss or another biologic drug may be recommended to the patient. In some embodiments, another biologic drug may comprise another drug for treating an immune mediate inflammatory disease, such as, for example ADA. If the subject has achieved TCZ>5 mg/L with 90% confidence, it is evaluated whether the subject's inter-dose interval can be elongated. For example, a model may evaluate whether the subject's inter-dose interval can be elongated to every other week. If yes, a dosing of every other week is initiated. If no, the subject's inter-dose interval remains once a week.

TABLE 9
TCZ parameter estimates

| Parameter | Parameter code | Monolix | Estimate |
|---|---|---|---|
| bioavailabilty | F | F | 0.80 |
| Absorption rate constant | Ka (day⁻¹) | ka_pop | 0.23 |
| Apparent Clearance | CL/F_pop (L/day) | Cl_pop | 0.216 |
| Volume of Distribution of | Vc/F_pop (L) | Vc_pop | 4.5 |
| Central Compartment | | | |
| Apparent | Q/F pop (L/day) | Q_pop | 0.274 |
| intercompartment | | | |
| clearance | | | |
| Apparent Volume of | Vp/F (L) | Vp_pop | 2.8 |
| Peripheral Compartment | | | |
| Max Velocity at High | Vmax (mg/day) | Vmax | 1.9 |
| Substrate Concentrations | | | |
| Affinity of the substrate | Km | Km | 0.34 |
| for the biologic drug | | | |
| Random effect on CL | Omega CL | OMEGA_CL | 0.248 |
| Random effect on Vc | Omega V | OMEGA_Vc | 0.236 |
| Random effect on Q | Omega Q | OMEGA_Q | NA |
| Random effect on Vp | Omega Vp | OMEGA_Vp | 0.396 |
| Random effect on Vmax | Omega Vmax | OMEGA_Vmax | 0.333 |
| Estimate of weight on | beta_Cl_logtWEIGHT | beta_Cl_logtWEIGHT | 0.51 |
| clearance | | | |
| Estimate of weight on | beta_V_logtWEIGHT | beta_V_logtWEIGHT | 0.68 |
| Volume | | | |
| | Proportional error | b | 0.10 |

$$C L = 0.2 \; 16 L / day ; Vc = 4.5 L ;$$

$$Q = 0. 2 \; 74 L / day ; Vp = 2.8 L ; ka = 0.23 1 / day$$

$$F = 0.8 \% ; \% MM = elimination V max = 19 mg / day ; Km = 0.34 , mg / L$$

$$\% IIV ( omega )$$

$$\% Not listed in Abdallah .$$

$$Consider values from Grey , N et al . JCP 2010$$

$$CL\_IIV = sqrt ( log ( 0.39 \hat{} 2 + 1 ) ) ; Vc\_IIV = sqrt ( log ( 0.37 \hat{} 2 + 1 ) ) ;$$

$$Q\_IIV = 0 ; Vp\_IIV = sqrt ( log ( 0.66 \hat{} 2 + 1 ) ) ;$$

$$ka\_IIV = 0 ; Vmax\_IIV = sqrt ( log ( 0.54 \hat{} 2 + 1 ) ) ; Km\_IIV = 0 ;$$

$$\% Covariates ( WT normalized to 70 KG individual$$

$$b\_CL \_wt = 0.51 ;$$

$$b\_Vc \_wt = 0.68 ;$$

### Example 5. Precision Dosing for Adalimumab with Clinical Decision Tool Involving Poor Prognostic Factor of Pharmacokinetic Origin (PPFPK)

In this example, a subject is treated with a biologic drug to treat an immune-mediated inflammatory disease, such as adalimumab (ADA). A physician wishes to know how to optimize the current ADA therapy regiment to maintain a desired trough level of ADA in the patient using a clinical decision tool that calculates the probability of clinical remission (“clinical remission status”) and a poor prognostic factor of pharmacokinetic origin (PPFPK). The PPFPK in this example, is calculated by adding the presence of accelerated clearance (=1), with the presence of levels below pre-specified threshold (=1), forming the PPFPK (e.g., both present=2, only one present=1, non present=0). The method may include one or more of the following steps:

Assaying a Sample from the Subject

A sample obtained from the subject is assayed to measure 1) a concentration of the ADA, 2) a concentration of anti-ADA antibodies, 3) a concentration of albumin, 4) a concentration of C-Reactive Protein (CRP). In this example, the sample is a blood sample, such as a capillary blood sample or a venous blood sample obtained by phlebotomy.

The concentration of ADA is measured by any one of the assays disclosed in Example 1.

The concentration of anti-ADA antibodies is measured by any one of the assays disclosed in Example 1.

The concentration of albumin is measured by any one of the assays disclosed in Example 1.

### Determine the Poor Prognostic Factor of Pharmacokinetic Origin (PPFPK)

The clinical decision tool in this example takes into consideration “poor prognosis factor” (PPF) of pharmacokinetic (PK) origin (PPFPK), which is the estimated clearance of the drug using the concentration of albumin measured in the sample and the weight of the subject as covariates, together with dose given drug levels and antidrug antibody status which is indicative of the subject's propensity to clear the ADA from their system.

The concentration of albumin and the subject's weight are input into a the model disclosed herein to determine the PPFPK.

### Evaluating Inflammatory Status

The inflammatory status of the subject was evaluated by evaluating CRP. In this example, the level of CRP indicative of inflammation is below a cutoff of about 3 mg/L. In other examples the cutoff may be below about 3 to about 5 mg/L, or any number in between these two values.

### Evaluating the Clinical Remission Status of the Subject

Clinical remission status of the subject was determined by looking at CRP levels measured in the biological sample obtained from the subject, and patient reported outcomes. In this example, the level of CRP indicative of clinical remission is below a cutoff of about 3 mg/L. In other examples the cutoff may be below about 3 to about 5 mg/L, or any number in between these two values. Patient reported outcomes may be reported using a score disclosed herein, such as, for example PRO2, CDAI, or DAS. In this example, a PRO2>8 (corresponding to CDAI of 150 points) was used as the cutoff, below which, is indicative of clinical remission.

### Adjusting the Inter-Dose Interval of ADA for the Subject

The inter-dose interval for ADA is adjusted based on the clinical remission status and presence or absence of the PPFPK determined above. In the presence of CRP based disease remission status achieved, the value based pricing point (this is also the clinical utility checkpoint) is initiated to iteratively evaluate an elongation the inter-dose interval to recommend an elongation up to every four weeks in the absence of forecasted PPF of pharmacokinetic origin. The “clinical utility checkpoint” as used herein refers to the point where the biological sample is collected and is interrogated against prior information to provide dosing guidance through the test report. In the absence of CRP based clinical remission status achieved, the value based pricing is initiated iteratively to evaluate a shortening of the inter-dose interval that remediates the PPFPK. If there is no possibility to achieve exposure commensurate with disease control (e.g., to remediate the PPFPK), SIP or JAK small molecules are initiated owing to the accelerate clearance for the class of monoclonal antibodies irrespective of their targeted cytokine. In the absence of both CRP based clinical remission status achieved and PPFPK (the exposure is commensurate with disease control), S1P or JAK small molecules is initiated.

This dosing tool incorporates the elongation in the dosing interval in the absence of inflammation and symptoms, only which corresponds to the achievement of a CRP based clinical remission status constructed from a clinical disease activity Index (lower than 8 point, PRO2 of CDAI below 150 points, etc. HBI<5 points points), in the absence of inflammation (CRP<3 mg/L or <5 mg/L). Swollen and tender joint counts from the EMR are collected with patient and physician assessment of disease activity. The poor prognostic factors that can sometime arise in the presence of active disease status can be identified by the clinical decision tool, and combined with the CRP based clinical remission status achieved. As presented in the Table 11 below:

### Example 6. Precision Dosing for Tocilizumab (TCZ) with Clinical Decision Tool Involving PPFPK

Referring to FIG. 10, in this example, a patient with rheumatoid arthritis (RA) or cytokine release syndrome is given therapy every other week unless the patient weighs more than 100 Kg, in which case the patient is given therapy weekly. Described herein is a clinical decision tool that incorporates together PPFPK to decide to (i) shorten or elongate the inter-dose interval of TCZ, or (ii) terminate TCZ therapy to initiate adalimumab treatment. This clinical decision tool utilizes as covariates: 1) the weight of the patient, 2) achievement of exposure commensurate with disease control, and 3) Body mass index (BMI).

A sample obtained from the patient is assayed to measure 1) a concentration of the TCZ, 2) a concentration of anti-TCZ antibodies, 3) a concentration of albumin, 4) a concentration of C-Reactive Protein (CRP), and 5) concentration of interleukin 6 (IL-6). In this example, the sample is a blood sample, such as a capillary blood sample or a venous blood sample obtained by phlebotomy.

The concentration of TCZ, anti-TCZ antibodies, albumin, and IL-6 are measured by one or more assays disclosed in Example 1.

BMI of the patient is obtained by weighing the patient. The patient, in this example weigh himself at home and inputs the weight into a mobile application of his electronic device, such as the mobile application described herein.

A non-linear mixed effects modeling (NLME) for pharmacometrics (e.g., Monolix, NONMEM, MATLAB software) is used model pharmacokinetics (PK) and pharmacodynamics (PD) of TCZ using the parameter estimates provided in Table 11. The conditional distribution of the parameter is estimated using a probabilistic machine learning based tools (e.g., Bayesian machine learning algorithm). The outcome PK variable is calculated by sampling from these conditional distributions. The clinical utility checkpoint estimates the probability to achieve commensurate exposure for the condition to be treated (rheumatoid arthritis or cytokine release syndrome). In this example, a threshold above 5 mg/L of TCZ is selected by the pharmacy benefit manager, which is reasonable given the ACR Appropriateness Criteria® 50 performances, at that level. The machine-based tool calculates the probability to be above that threshold with confidence. In any situation where the exposure threshold cannot be achieved, the clinical decision tool will recommendation to initiate adalimumab (ADA) instead of TCZ. Alternatively, if the patient presents both PPFPK origin (for example TCZ below pre-specified threshold in the presence of accelerated clearance 0.216 L/day, then small molecules are initiated, such as inhibitors of SIP or JAK disclosed herein.

If the patient is less than 100 Kg, and the clinical utility checkpoint has been achieved after 10 weeks of treatment, then the clinical decision tool recommends to elongate the inter-dose interval to every three weeks on the basis of the confidence to achieve threshold concentration commensurate with disease control. If that outcome is not met, logically, evaluate every week dosing possibility, most importantly in the presence of inflammation and symptoms, IL-6, CRP (C-Reactive Protein) and Patient reported outcomes (PRO2).

If the patient is more than 100 Kg, and the clinical utility checkpoint is met (high probability to achieve commensurate exposure for the condition to be treated), then the clinical decision tool will recommend an elongation of the inter-dose interval of TCZ, or to continue therapy at the current inter-dose interval. By contrast, where the clinical utility checkpoint is not met (low probability to achieve commensurate exposure for the condition to be treated), the clinical decision tool will recommend to initiate ADA treatment. TCZ can be administered at a dose of 162 mg injected and at inter-dose intervals between 7 and 28 weeks. Pre-dose trough concentration (Ctrough) is defined as the drug concentration observed at the last planned timepoint prior to dosing.

TABLE 13
TCZ Clinical Decision Tool in the United States

| | | START Every two | Every | Every |
|---|---|---|---|---|
| | Every week | weeks USA Indication | Three weeks | Four weeks |
| Pharmacokinetic | >90% likelihood of | >90% likelihood of | >90% likelihood of | >90% likelihood of |
| (“PK”) | Ctrough >5 mg/L | Ctrough50 mg/L | Ctrough >5 mg/L | Ctrough >25 mg/L |
| Outcome | | | | |
| PK Outcome | Yes, Evaluate every | Yes, Evaluate every | Yes, evaluate every | Yes, continue |
| achieved | two weeks regimens in | three weeks regimen | four weeks regimen | treatment |
| | the absence of | in the absence of | in the absence of | No: switch to every |
| | inflammation and | inflammation and | inflammation and | three weeks in the |
| | symptoms | symptoms | symptoms | presence of |
| | No: increase to 162 mg | No: switch to every | No: switch to every | inflammation and |
| | twice a week in the | Week in the presence | two weeks in the | symptoms |
| | presence of | of inflammation and | presence of | |
| | inflammation and | symptoms | inflammation and | |
| | symptoms | | symptoms | |

TABLE 14
TCZ Clinical Decision Tool In Europe

| | START: | | | |
|---|---|---|---|---|
| | Every week | | | |
| | European Indication | Every Two weeks | Every Three weeks | Every Month |
| Conditional | >90% likelihood of | >90% likelihood of | >90% likelihood of | >90% likelihood of |
| Distribution of | Ctrough >5 mg/L | Ctrough >5 mg/L | Ctrough >5 mg/L | Ctrough >5 mg/L |
| the parameter | | | | |
| estimates | | | | |
| PK Outcome | Yes, Evaluate every | Yes, Evaluate every | Yes, evaluate every | Yes, continue |
| achieved | two weeks in the | three weeks regimen | four weeks regimen | treatment |
| | absence of inflammation | in the absence of | in the absence of | |
| | and symptoms. | inflammation and | inflammation and | |
| | | symptoms. | symptoms. | |
| | NO: increase to 162 mg | No: switch to weekly | No: switch to every | No: switch to every |
| | twice a week in the | in the presence of | two weeks in the | three weeks in the |
| | presence of inflammation | inflammation and | presence of inflammation | presence of inflammation |
| | and symptoms | symptoms | and symptoms | and symptoms |

TABLE 15
TCZ Clinical Decision Tool In United States

| | | START | | |
|---|---|---|---|---|
| | START | Every two weeks | | |
| | Every Week (>100 Kg) | USA Indication (<100 Kg) | Every Three weeks | Every Four weeks |
| PK Outcome | 100% likelihood of | 100% likelihood of | 100% likelihood of | 100% likelihood of |
| | Ctrough >5 mg/L | Ctrough >5 mg/L | Ctrough >5 mg/L | Ctrough >5 mg/L |
| PK Outcome | Yes, Evaluate every | Yes, Evaluate every | Yes, evaluate every | Yes, continue |
| achieved | two weeks regimens in | three weeks regimen | four weeks regimen | treatment |
| | the absence of | in the absence of | in the absence of | |
| | inflammation and | inflammation and | inflammation and | |
| | symptoms, elongation | symptoms, elongation | symptoms | |
| | No: increase to 162 mg | No: switch to | No: switch to | No: switch to |
| | twice a week in | every Week in | every two weeks in | every three weeks in |
| | the presence of | the presence of | the presence of | the presence of |
| | inflammation and | inflammation and | inflammation and | inflammation and |
| | symptoms | symptoms | symptoms | symptoms |

TABLE 16
TCZ Clinical Decision Tool In Europe

| | START: | | | |
|---|---|---|---|---|
| | Every week | | | |
| | European Indication | Every Two weeks | Every Three weeks | Every Month |
| Conditional | 100% likelihood of | 100% likelihood of | 100% likelihood of | 100% likelihood of |
| Distribution of | Ctrough >5 mg/L | Ctrough >5 mg/L | Ctrough >5 mg/L | Ctrough >5 mg/L |
| the parameter | | | | |
| estimates | | | | |
| PK Outcome | Yes, Evaluate every | Yes, Evaluate every | Yes, evaluate every | Yes, continue |
| achieved | two weeks in the | three weeks regimen in | four weeks regimen | treatment |
| | absence of | the absence of | in the absence of | |
| | inflammation and | inflammation and | inflammation and | |
| | symptoms. | symptoms. | symptoms. | |
| | NO: increase to 162 | No: switch to weekly | No: switch to every | No: switch to every |
| | mg twice a week in | in the presence of | two weeks in the | three weeks in |
| | the presence of | inflammation and | presence of | the presence of |
| | inflammation and | symptoms | inflammation and | inflammation and |
| | symptoms | | symptoms | symptoms |

### Example 7. Clinical Decision Tool for Determine Value Based Pricing of TCZ

A total of 1000 patients with RA have been simulated using the parameter estimates provided in the Table 17. This consists of 342 patients weighing above 100 Kg and 658 patients weighing below 100 Kg. In this example all patients weighing above 100 Kg start 162 mg SC weekly, and all patients weighing less than 100 Kg start at 162 mg every other week (EOW). At steady state, the trough estimate before TDM is 12.7 mg/L for patients weighing 100 Kg of more as compared to 5.7 mg/L in patients weighing 100 Kg or less. For a year of treatment after the dose regimen to reach steady state is given the total costs for the 1000 patients is $39 MM with 47% patients achieving target concentration.

TABLE 17
Population estimate baseline

| | | | | | | | | | | IItrough | Pct |
|---|---|---|---|---|---|---|---|---|---|---|---|
| | | Avg | Avg | Avg | Avg | | Avg | Avg | Avg | fixed | target > |
| II | N | Ka | Wt | CL | Vc | Q | Vp | Vmax | Km | dose | 5 mg/L |
| 162 SC, 7 d | 342 | 0.23 | 125 | 0.306 | 7.2 | 0.274 | 3.23 | 2.1 | 0.34 | 12.7 | 0.65 |
| 162 SC, 14 d | 658 | 0.23 | 68 | 0.226 | 4.7 | 0.274 | 3.49 | 2.1 | 0.34 | 5.7 | 0.37 |
| Total | 1000 | 0.23 | 88 | 0.253 | 5.6 | 0.274 | 3.40 | 2.1 | 0.34 | 8.1 | 0.465 |

Avg Ka refers to the average rate at which TCZ enters the system of the subject.
Avg Wt refers to the population average weight.
Avg CL refers to the average clearance.
Avg Vc refers to the average volume of distribution of the central compartment.
Q refers to the intercompartment clearance.
Avg Vp refers to volume of the peripheral compartment.
Avg Vmax refers to the average maximum velocity at high TCZ concentrations.
Avg Km refers to the average affinity of TCZ to bind IL-6.
IItrough fixed dose refers to the trough concentration of TCZ per the drug label (without using the clinical decision tool disclosed herein to optimize the dose). In the USA, the inter-dose interval is every two weeks; in Europe, the inter-dose interval is every week.
Pct target > 5 mg/L refers to probability to have levels of TCZ that are above 5 mg/L.

TABLE 18
Total drug cost, Standard of Care

| | | | | | C | Cl | C > |
|---|---|---|---|---|---|---|---|
| | N | Cost | N | Total cost | avg | avg | 5 mg/L |
| <100 Kg | 658 | $1,133 | 26 | $19,381,311 | 5.7 | 0.226 | 37% |
| >100 Kg | 342 | $1,133 | 52 | $20,147,138 | 12.7 | 0.306 | 65% |
| Total | 1000 | $1,133 | 35 | $39,528,449 | 8.1 | 0.253 | 47% |

TABLE 19
Fixed dosing q1w (every week) or q2w (every two weeks)
The clinical decision model adapts dose and inter-dose interval
to achieve desired pharmacokinetics results with smaller doses
or less frequent dosing. The simulation established the following
performance characteristics:

| Inter-dose interval | <100 Kg | >100 Kg | Grand Total |
|---|---|---|---|
| 2q1w (twice every week) | 74 | 120 | 194 |
| q1w (once every week) | 341 | 173 | 514 |
| q2w (once every two weeks) | 181 | 40 | 221 |
| q2w (once every three weeks) | 48 | 9 | 57 |
| q4w (once every four weeks) | 14 | | 14 |
| Grand Total | 658 | 342 | 1000 |

In the group of 658 patients presenting with weight below 100 Kg and started at every other week regimen, a total of 243 of them achieve target concentration. Of those 243 subjects, elongation to every three weeks treatment regimens results into exposure below 5 mg/L in 181 patients and as such no change in the dose in proposed by the dosing tool. Alternatively, elongation of the inter-dose interval to every 3 weeks and every four weeks will results in exposure above 5 mg/L in 48 and 14 patients, respectively. In contrast, for the 415 subjects that do present with exposure below 5 mg/L at trough, inter-dose interval shortening to weekly schedule will result in adequate exposure above 5 mg/L in 341 patients.

In the group of 342 patients presenting with weight above 100 Kg, and starting at the weekly dosing regimen, a total 120 patients do not achieve target exposure while 222 patients do. Of the 222 subjects the dosing tool suggest an elongation in the dose interval to two weeks and three weeks with maintenance of exposure above threshold in 40 and 9 patients, respectively with no change in the dose interval in 173 patients. A total of 74 patients benefit from two injections per week to maintain levels above threshold.

TABLE 20
Exposure achieved after dose change

| | | |
|---|---|---|
| Inter-dose Interval | Weight <100 Kg | Weight >100 Kg |

| | | | | |
|---|---|---|---|---|
| 2q1 | 34.9 | (n = 74) | 29.1 | (n = 120) |
| q1 | 25.7 | (n = 341) | 21.7 | (n = 173) |
| q2 | 13.4 | (n = 181) | 10.7 | (n = 40) |
| q3 | 8.7 | (n = 48) | 8.3 | (n = 9) |

| | | | |
|---|---|---|---|
| q4 | 9.5 | (n = 14) | 0 |

The results of the clinical decision tool associated with enhanced exposure at the expense of additional costs corresponding to the number of doses given to achieve exposure commensurate with disease control.

TABLE 21
Total drug cost, TDM

| | | | | | C | Cl | C > |
|---|---|---|---|---|---|---|---|
| | N | Cost | N | Total cost | avg | avg | 5 mg/L |
| <100 Kg | 658 | $1,133 | 49 | $36,740,054 | 21.8 | 0.226 | 99% |
| >100 Kg | 342 | $1133 | 72 | $28,041,046 | 22.6 | 0.306 | 95% |
| Total | 1000 | $1,133 | 57 | $64,781,099 | 22.1 | 0.253 | 97% |

In that group, the cost associated with enhanced pharmacokinetic intervention is $64 MM as compared to $39 in the standard dosing approach with 22.1 mg/L as compared to 8.1 m/L.

TABLE 22
Cost Reduction Example

| | | Clinical | | |
|---|---|---|---|---|
| | Standard | Decision | | |
| | cost | Tool value | | |
| Setting | 162 mg SC | base pricing | Prorated | Discount |
| Standard dosing | $1133 | $1133 | 100% | 0% |
| Elongation q3w, | $1133 | $1133 | 100% | 0% |
| q4w | | | | |
| Shortening q2w | $1133 | $566 | 50% | 50% |
| to q1w | | | | |
| Shortening q1w | $1133 | $283 | 25% | 75% |
| to 2q1w | | | | |

TABLE 23
Clinical Decision Tool Value Based Pricing (VBP)

| | | | | Clinical |
|---|---|---|---|---|
| | | Fixed | Fixed | Decision |
| Intervention | N | dosing | TDM | Tool VBP |
| Double dose, | 194 | $9,248,832 | $26,666,485 | $9,248,832 |
| shortening | | | | |
| shortening II | 341 | $10,044,114 | $20,088,228 | $10,044,114 |
| standard dosing | 354 | $15,522,722 | $15,522,722 | $10,191,388 |
| Elongation II | 111 | $4,712,781 | $2,503,665 | $1,030,921 |
| Grand Total | 1000 | $39,528,449 | $64,781,099 | $30,515,256 |

Cost of the clinical decision tool, twice a year, $1500 or $1.5 MM costs for the plan. Saving $29 MM per year for the health plan.

TABLE 24
Clinical Decision Tool Value-Based Pricing and Doses

| | | Fixed | | |
|---|---|---|---|---|
| | | dosing | PPK | |
| | | Number | Intervention | Dose |
| | N | of Dose | Nb dose | Difference |
| Double dose with | 194 | 8164 | 23539 | 15375 |
| shortening | | | | |
| shortening II | 341 | 8866 | 17732 | 8866 |
| standard dosing | 354 | 13702 | 13702 | 0 |
| Elongation II | 111 | 4160 | 2210 | −1950 |
| Grand Total | 1000 | 34892 | 57183 | 22291 |

TABLE 25
Clinical Decision Tool VBP and Exposure

| | | Fixed | Clinical | | |
|---|---|---|---|---|---|
| | | dosing | Decision | Fixed | Clinical |
| | | levels | Tool based | dosing | Decision |
| | N | mg/L | Levels mg/L | Cost | Tool VPB |
| Less than 100 Kg | 658 | 5.7 | 21.8 | $19,381,311 | $12,901,237 |
| More than 100 Kg | 342 | 12.7 | 22.6 | $20,147,138 | $17,614,018 |
| Grand Total | 1000 | 8 | 22 | $39,528,449 | $30,515,256 |

this example, the clinical decision tool value based pricing comprises a cost per patient per year per mg/L at trough.

TABLE 26
Cost Per Patient Per Year Per mg/L Exposure

| | | Clinical |
|---|---|---|
| | Fixed | Decision |
| | dosing | Tool |
| less than 100 Kg | $5,205 | $901.44 |
| More than 100 Kg | $4,640 | $2,335.31 |
| Total | $4,901 | $1,383 |

FIG. 11 provides anon-limiting workflow of the clinical decision tool for value-based pricing for TCZ. TCZ levels and albumin are measured in a sample from the subject. Weight and BMI are also obtained for the subject, either by self-reporting via a mobile application on the subject's personal electronic device or by accessing the electronic medical records for the subject. A Markov chain Monte Carlo simulation and Metropolis Hastings algorithm is applied to estimate a plurality of conditional distributions of the parameter estimates for the subject. In this case, the parameters are Vc, Q, Vp and Vmax. An iterative process is performed to estimate the likelihood of achieve a trough concentration of TCZ of 5 mg/L with 50% confidence. The clinical decision tool recommends an elongation of the inter-dose interval if the likelihood of achieving the trough concentration of higher than 5 ml/L is achieved with above 50% probability. The clinical decision tool recommends shortening of the inter-dose interval if the likelihood of achieving the trough concentration of higher than 5 ml/L is achieved with below 50% probability. This is evaluated for inter-dose intervals of every two weeks (“q2w”), every three weeks (“q3w”) and every four weeks (“q4w”). The value-based pricing sequence is initiated to provide the value-based pricing and doses.

Sampling from the Conditional Distribution

Sampling from the plurality of the conditional distribution and constitute the Bayesian Estimate Sustaining Trough concentration above or below threshold.

### Example 7. Optimizing Inter-Dose Interval or Dose of ADA Using Clinical Decision Tool

This example provides a method for treating Rheumatoid arthritis in a patient in need thereof, by optimizing the dose and inter-dose interval of ADA using the clinical decision tool described herein, as shown in FIGS. 12A-12B.

Obtain a Sample from the Patient

A sample is obtained from the patient. In this case, the sample is a blood sample obtained by phlebotomy or micro sampling capillary collection device. A concentration of C-reactive protein, ADA levels, anti-ADA antibodies, and albumin are measured in the sample using methods disclosed in previous examples.

### Medical Records of the Patient

Electronic medical records of the patient are obtained, which include estimates of the global assessment, weight, assessment of disease activity (e.g., swollen and tender joint counts, global assessment). These records can be obtained from the patient directly via a mobile application on the patient's personal electronic device. For example, the patient can input the weight, symptoms (rectal bleeding, weight loss, appetite, etc.) into the application on their personal electronic device. These records can also come from the patient's doctor, such as the global assessment of disease activity.

### Setting a Threshold of Bayesian Estimate

A threshold of Bayesian estimate sustain trough concentration commensurate with effective disease control and remission is pre-set. The threshold is set by a clinician guideline, or the ordering physician.

### Determining Probability of Clinical Remission

A probability of achieving clinical remission is determined using the CDAI of lower than 2.8 points in combination with a concentration of C-Reactive Protein (CRP) in the patient's sample that is below 3 mg/L.

### Determining the Poor Prognostic Factor of Pharmacokinetic Origin (PPFPK)

The presence of a poor prognostic factor of pharmacokinetic origin (PPFPK) that corresponds to the Bayesian estimate sustaining trough below the predetermined threshold is determined. In this example, a presence of accelerated clearance of the biologic drug (PPF1) and a presence of low drug levels (below a pre-determined cutoff concentration) (PPF2) means that the subject has a presence of the PPFPK. In this example, the PPF1 cutoff for clearance (CL) is >0.216 L/day, and the PPF2 cutoff is less than 5 mg. The cutoff's for ADA were derived from optimal Youden index using the methodologies disclosed in Example 22.

### Combining Probability of Clinical Remission and Bayesian Estimate Sustaining Trough Concentration

Combining the clinical remission status with Bayesian Estimate Sustaining Trough concentration above threshold to inform on elongation, shortening or no change in the inter-dose interval.

### Estimate Conditional Distribution of Parameter Estimates

Applying Markov chain Monte Carlo simulation and Metropolis Hastings algorithm to estimate a plurality of conditional distributions of the parameter estimates for the patient, which are provided in Table 5.

Sampling from the Conditional Distribution

Sampling from the plurality of the conditional distribution and constitute the Bayesian Estimate Sustaining Trough concentration above or below threshold.

### Incorporating Bayesian Estimate Sustaining Trough Concentration Above or Below the Threshold to Determine Value-Based Pricing

Value-based pricing for ADA is determined using the methods described above in previous examples. This value-based pricing is based, at least in part, on evidence from the clinical decision tool that the dose and inter-dose interval recommended for ADA will provide the minimum effective concentration to achieve the desired threshold concentration of ADA in the patient to treat the patient's disease. The clinical decision tool will recommend to prolong the inter-dose interval, in the presence of the Bayesian estimate sustain trough concentration above the predetermined threshold and disease remission status achieved above. Whereas the clinical decision tool will recommend to shorten the inter-dose interval in the presence of Bayesian estimate sustaining trough concentration below the predetermined threshold, and absence of disease remission status achieved above. The value-based pricing will suggest a discounted price to reduce the costs associated with the shortening of the inter-dose interval of ADA. However, in the presence of PPKPK, and in the absence of achievement of Bayesian Estimate sustaining Trough concentration above the predetermined threshold and clinical remission, the clinical decision tool will recommend initiating TCZ.

FIGS. 12A-12B provide a non-limiting workflow of the clinical decision tool for value-based pricing for ADA. A sample (“specimen”) is obtained from a subject. ADA levels and anti-ADA antibodies are measured in the sample from the subject using a homogeneous mobility shift assay (HMSA). Albumin and CRP are measured in the sample using HMSA. Weight and BMI are also obtained for the subject, either by self-reporting via a mobile application on the subject's personal electronic device or by accessing the electronic medical records for the subject. A Markov chain Monte Carlo simulation and Metropolis Hastings algorithm is applied to estimate a plurality of conditional distributions of the parameter estimates for the subject. In this case, the parameters are clearance and volume. An iterative process is performed to estimate the likelihood of achieve a trough concentration of ADA of 7.5 mg/L with 90% confidence. The clinical decision tool recommends an elongation of the inter-dose interval if the likelihood of achieving the trough concentration of higher than 7.5 ml/L is achieved with above 90% probability. The clinical decision tool recommends shortening of the inter-dose interval if the likelihood of achieving the trough concentration of higher than 7.5 ml/L is achieved with below 50% probability. This is evaluated for inter-dose intervals of every two weeks (“q2w”), every three weeks (“q3w”) and every four weeks (“q4w”). If the trough concentration of higher than 7.5 ml/L cannot be achieved with a probability of 90%, then the clinical decision tool recommends administering a different therapeutic agent, such as tocilizumab (TCZ). The value-based pricing sequence is initiated to provide the value-based pricing and doses.

### Example 8. Workflow of Clinical Decision Tool for a Biologic Drug

A physician wishes to know whether to maintain or elongate an inter-dose interval of a biologic drug in a treatment regimen for patient having a an immune-mediated inflammatory disease. As shown in FIGS. 13A, the clinical decision tool value-based pricing sequence is initiated. Electronic medical records (EMR) data is obtained either from the patient or the physician's medical records. In this example, if the patient has RA, then the swollen joints (SJ) and tender joints (TJ) and disease activity score (DAS) are obtained, as disclosed in detail elsewhere herein. A sample is obtained from the subject and delivered to a medical laboratory for processing and analysis. In this example, the sample is a blood sample collected by phlebotomy or a finger prick device, however, several suitable methods of obtaining a blood sample can be used. An active disease status is determined using the Clinical Disease Activity Index (CDAI) based disclosed herein. A CDAI of lower than 2.8 points suggests that the patient is in clinical remission. The inflammatory status of the patient is determined based on the concentration of C-Reactive Protein (CRP) in the patient's blood sample. A concentration below 3 mg/L suggests the patient is in clinical remission. If, based on CRP and the CDAI, the patient is predicted to achieve clinical remission, the clinical decision tool initiates an “elongation-based sequence” to elongate the inter-dose intervals of the current biologic drug. If, based on CRP and CDAI, the patient is predicted to not achieve clinical remission, the clinical decision tool initiates “interval shortening-based sequence” to shorten the inter-dose interval. Either sequence is performed to provide a recommended dose and inter-dose interval for the biologic drug according as demonstrated in previous examples. The recommendation in this example is sent to the physician's office.

The clinical decision tool also takes into account the probability that the patient will achieve a desired trough concentration of the biological drug (“threshold”) as determined using a Markov Chain Monte Carlo (MCMC) simulations using the patient's clearance, body weight as covariates. FIG. 13B provides anon-limiting illustration of how this works for two populations of patients, each of which are treated for 52 weeks after the initial phase of dosing (either every two weeks (“q2w”) for patients less than 100 Kg or one a week (“q1w”) for patients weighing more than 100 Kg).

Individual pharmacokinetic parameters are estimated using a combination of non-linear mixed effect models (NONMEM) and prior information from an hybrid of the two previously reported pharmacokinetic model with modifications to add a 10% proportional error model constants. The model employs 1^{st} order absorption with two compartment and linear nonlinear Michaelis Menten with random effects on clearance (Cl), volume of distribution (central, [V1] and peripheral [V2]) and Vmax. Covariates consists of patient weight (on Cl, V1). For the patients, biologic drug levels and weight were used to estimate the conditional distribution of the individual pharmacokinetic parameters. The conditional distribution represents the uncertainty of the individual parameters given the observations collected, and the prior information. Conditional distributions of the individual pharmacokinetic parameters (Cl, V1, V2 and Vmax) were generated for the subject using Markov Chain Monte Carlo (MCMC) simulations (Metropolis Hastings algorithm), and sampling (n=100) from those distributions were used to estimate the probability that exposure will achieve the desired threshold of drug concentration, e.g., 5 micrograms per milliliter (μg/mL). If the probability is low (<50%), then the interval shortening-based sequence is initiated. If the probability is high (>90%), the elongation-based sequence is initiated. In this example, the probability was low, so the clinical decision tool recommends that the inter-dose interval be shortened. That recommendation is communicated to the pharmacy and stored in a data store in the electronic medical records (EMR) for that patient. The test results of the combined workflow of FIG. 13A and FIG. 13B are communicated to the physician, and used as a basis to initiate a treatment regimen for the patient.

### Example 9. Poor Prognostic Factor of Pharmacokinetic Origin (PPFPK) Based on Biologic Drug Clearance Estimates and Biologic Drug Levels

Samples obtained from three cohorts of patients with inflammatory bowel disease (IBD) being treated with infliximab (IFX) were evaluated to determine whether clearance estimates alone for IFX or the combination of IFX levels in the sample and the estimated clearance of IFX were better predictors of clinical remission in these patients. Table 27 provides the study information. Table 28 provides details for three induction doses of IFX administered to the patients in each study.

TABLE 27
Study Information

| | Study1 | Study2 | Study | overall |
|---|---|---|---|---|
| Number of Patients | 56 | 114 | 189 | 359 |
| Gender (female) | 39% (16/41) | 59% (57/97) | 46% (75/164) | 49% (171/359) |
| Age (years), SD | 13.3 ± 3.4 | 35.5 ± 14.0 | 16.3 ± 7.0 | 21.6 ± 13.0 |
| % Pediatrics | 95% (53/56) | 0% (0/114) | 77% (146/189) | 55% (199/359) |
| CD % (of total) | 69.6% | 100% | 72.4% | 80.8% |
| Clearance (week 0) | 0.228 | 0.292 | 0.287 | 0.278 |
| L/day | (0.195-0.252) | (0.258-0.328) | (0.236-0.354) | (0.236-0.331) |
| Full Remission | 10% | 13% | 43% | 29% |

“CD %” refers to % of patients having Crohn's disease. “SD” refers to standard deviation.
Study 1 patients were recruited from Cedars-Sinai Medical Center.
Study 2 patients were recruited from Study Investigating Tailored Treatment With Infliximab for Active Crohn's Disease (TAILORIX); ClinicalTrials.gov Identifier: NCT01442025.

Study 3 patients were recruited from the Proactive Infliximab Optimization Using a PK Dashboard Versus SOC in Patients With Crohn's Disease: The OPTIMIZE Trial.

TABLE 28
Induction Of IFX Administered To Patients In Each Study

| | Study1 | Study2 | Study3′ | Overall |
|---|---|---|---|---|
| | Standard | DIS, symptoms, | DIS, targeted | All |
| | Dose | markers | levels | interventions |
| Clearance (week 0) L/day | 0.228 | 0.292 | 0.287 | 0.278 |
| | (0.195-0.252) | (0.258-0.328) | (0.236-0.354) | (0.236-0.331) |
| ATI (>3.1 U/mL) | 0% | 0% | 0% | 0% |
| Dose#1 (Week2) | 32 | 114 | 185 | 33 |
| Dose mg/Kg | 5.0 | 5.0 | 5.1 | 5.0 |
| | (5.0-5.0) | (5.0-5.0) | (5.0-10.0) | (5.0-5.2) |
| Weight Kg | 47 | 64 | 48 | 54 |
| | (34-55) | (57-76) | (36-63) | (42-66) |
| Clearance (L/day) | 0.216 | 0.292 | 0.273 | 0.273 |
| | (0.188-0.258) | (0.239-0.354) | (0.210-0.363) | (0.214-0.353) |
| ATI Status | 7.1% | 1.8% | 1.1% | 2.2% |
| Dose#2 (Week6) | 53 | 110 | 175 | 338 |
| Dose mg/Kg | 5.0 | 5.0 | 5.1 | 5.0 |
| | (5.0-5.0) | (5.0-5.0) | (5.0-10.0) | (5.0-6.0) |
| Weight Kg | 49 | 66 | 50 | 55 |
| | (36-55) | (59-76) | (37-64) | (43-67) |
| Clearance (L/day) | 0.216 | 0.265 | 0.241 | 0.246 |
| | (0.160-0.333) | (0.200-0.320) | (0.183-0.318) | (0.185-0.320) |
| ATI Status | 23.2% | 4.4% | 5.9% | 8.1% |
| Dose#3 (Week14) | 47 | 101 | 171 | 319 |
| Dose mg/Kg | 5.0 | 5.0 | 9.9 | 5.0 |
| | (5.0-5.0) | (5.0-5.0) | (5.0-10.0) | (5.0-9.9) |
| Weight Kg | 50 | 67 | 51 | 56 |
| | (37-55) | (59-78) | (38-65) | (44-69) |
| Clearance (L/day) | 0.274 | 0.233 | 0.215 | 0.221 |
| | (0.147-0.263) | (0.188-0.283) | (0.170-0.292) | (0.171-0.289) |
| ATI Status | 23.6% | 10.9% | 6.0% | 10.3% |

“DIS” refers to dose intensification strategy. “ATI” refers to antidrug antibodies to Infliximab.

Whether or not patients receiving IFX achieved remission was analyzed in patients with accelerated clearance of IFX (above 0.25 L/day) and patients with normal clearance (below 0.25 L/day). As shown in FIG. 14, remission was observed in fewer patients having accelerated clearance of IFX than those patients with normal clearance. Without being bound by any particular theory, this suggests that faster clearance rates of IFX contribute to lower rates of clinical remission of IBD in these patients.

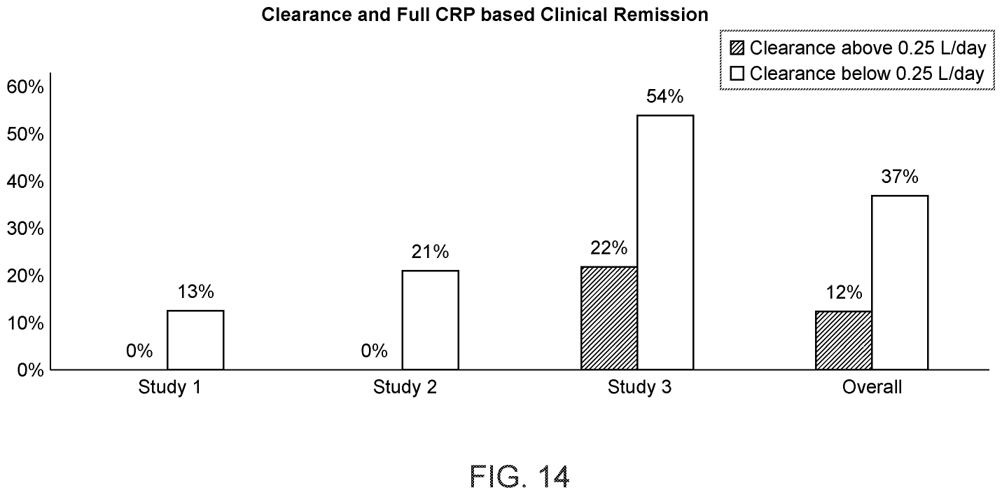

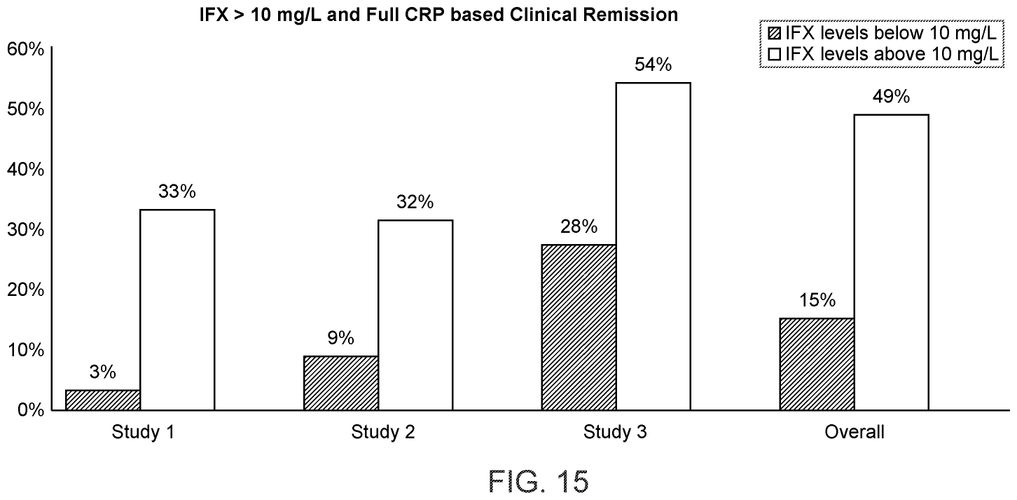

Whether or not patients receiving IFX achieved remission was analyzed in patients with concentrations of IFX in their blood below 10 mg/L or above 10 mg/L. As shown in FIG. 15, remission was observed in fewer patients having concentration of IFX below 10 mg/L than those patients with concentrations of IFX above 10 mg/L. Without being bound by any particular theory, this suggests that the biologic drug concentration (in this case, IFX) being below a certain threshold contributes to lower rates of clinical remission of IBD in these patients. In this example, the 10 mg/L threshold was derived from optimal Youden index that distinguished response or lack thereof, based on the clearance or the trough estimate.

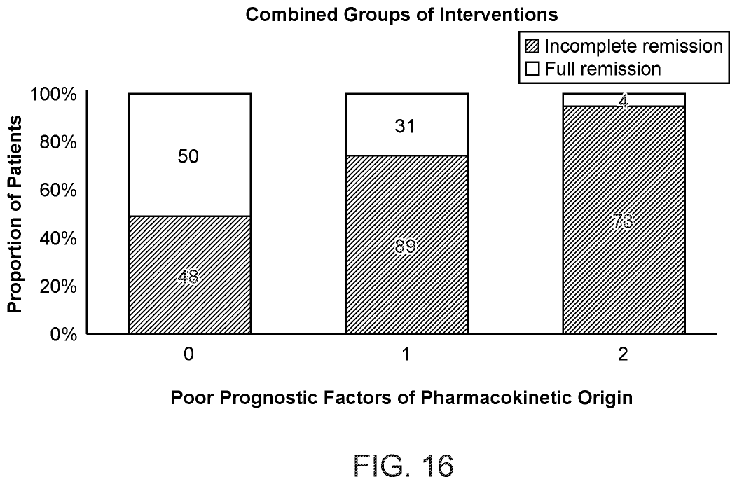

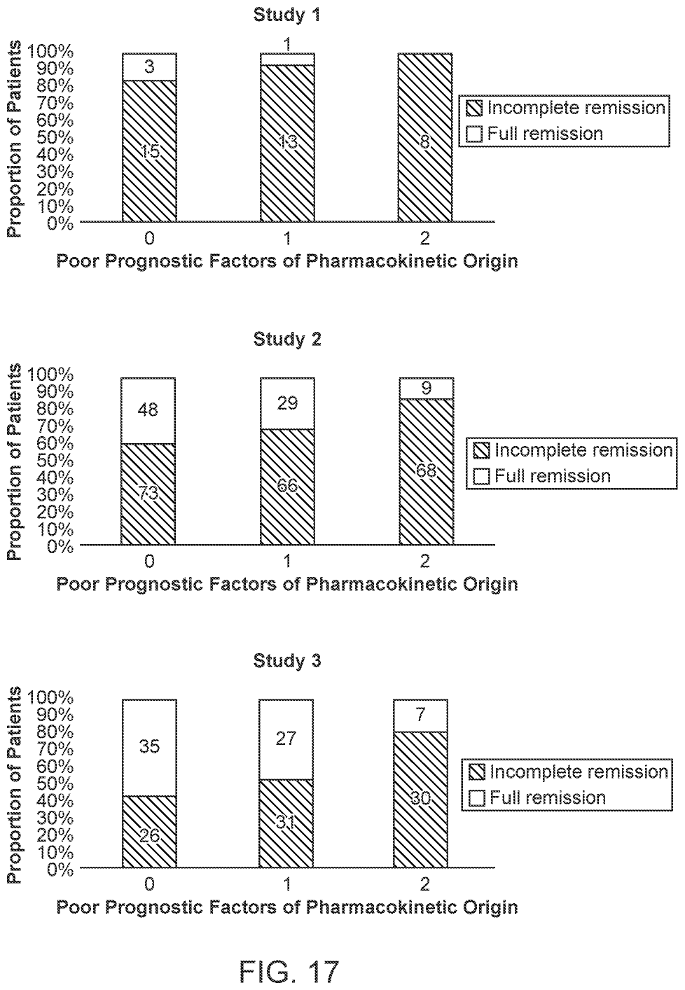

Based on the foregoing, the combination of using clearance rates of IFX and concentration of IFX in the patient's blood were used to predict clinical remission in these patients, as compared with using one or the other alone. As shown in FIG. 16, when clearance rates (above 0.25 L/day), alone are used (denoted with “0”) roughly half of the patients achieve clinical remission, and roughly 20% of patients achieve clinical remission when the concentration of IFX (below 10 mg/L) is used alone (denoted with a “1”). By contrast, when clearance rates (above 0.25 L/day) and concentration of IFX (below 10 mg/L) are used in combination as a poor prognostic factor of PK origin (PPFPK) (denoted with a “2”), roughly 4% of patients achieve clinical remission. Without being bound by any particular theory, this suggests that use of both clearance rates (above 0.25 L/day) and concentration of IFX (below 10 mg/L) may be a better predictor of clinical remission, than using one or the other alone. FIG. 17 shows that both clearance rates (above 0.25 L/day) and concentration of IFX (below 10 mg/L) provide a better predictor of clinical remission in each of the patient cohorts.

Thus, the PPFPK described above in previous examples may incorporate the clearance rate of the biologic drug, the concentration of the biologic dug, or a combination thereof. Moreover, this PP FPK is applicable to any biologic drug for treatment for any immune-mediated inflammatory disease disclosed herein. When used, either alone or in combination with the clinical decision tool described herein this PPFPK may support a prescription of a different type of therapeutic agent, such as a small molecule inhibitor (e.g., JAK inhibitor).

### Example 10. Clinical Decision Model for IFX Induction

The clinical decision tool for IFX in this example is configured to optimize an induction therapy for a subject with IBD disclosed herein. In this example, the IBD patients used in the reference population are not receiving IFX for 8 weeks, and have only received IFX as an induction in four doses at 0, 2, 4, and 6 weeks.

A sample obtained from the patient is assayed to measure 1) a concentration of the IFX, 2) a concentration of anti-IFX antibodies, 3) a concentration of albumin, 4) a concentration of C-Reactive Protein (CRP) using the HMSA assay disclosed in Example 1. In this example, the sample is a blood sample, such as a capillary blood sample or a venous blood sample obtained by phlebotomy. BMI of the patient is obtained by weighing the patient. The patient, in this example weigh himself at home and inputs the weight into a mobile application of his electronic device, such as the mobile application described herein. Prior information from the reference population of IBD subjects have only received IFX as an induction in four doses at 0, 2, 4, and 6 weeks is input into the model.

These measurements and prior information are input into the clinical decision tool described above, which utilizes Bayesian data assimilation using the parameter estimates provided in Table 10. The conditional distribution of the parameter is estimated using the Bayesian assimilation. The outcome PK variable is calculated by sampling from these conditional distributions. The clinical utility checkpoint estimates the probability to achieve commensurate exposure for inflammatory bowel disease (e.g., CD or UC).

If the estimated clearance rate of IFX is above 0.25 L/day and if the concentration of IFX is below 10 mg/L), then the clinical decision tool recommends switching the subject to a small molecule therapy, such as an inhibitor of JAK or S1P. Whereas, if the estimated clearance rate of IFX is below 0.25 L/day and the concentration of IFX is above 10 mg/L, then the clinical decision tool recommends an induction therapeutic strategy with IFX.

If the clinical decision tool recommends an induction therapeutic strategy with IFX, then the clinical decision tool recommends which dose and inter-dose interval to achieve a pre-specificized threshold concentration of IFX to treat the IBD. In this example, the pre-specified threshold is set by a physician or industry-wide standards.

### Example 11. Test Report for Clinical Decision Tool

The clinical decision tool described herein provides results in the form of a test report that may be transmitted to a physician or health care provider. The test report has three sections, which are illustrated here as a non-limiting example.

### Section 1: Patient Information at Last Infusion

Informed treatment management decisions begin with accurate and reliable information on your patient's current treatment regimen. Prominently displayed in the clinical decision tool biologic drug test report header, you can quickly reference key details of your patient's current infliximab treatment at the time of their last infusion prior to testing: (1) derived dose in mg/kg, (2) dose total in mg, (3) weight in kg, (4) dosing interval in weeks, (5) date of last infusion. FIG. 18A provides a non-limiting example of section 1 for IFX.

### Section 2: Current Results & Alternate Estimated IFX Concentrations

Referring to FIG. 18B, in the table on the left, the test report provides the patient's current IFX exposure as well as powerful insights into the trajectory of therapeutic drug levels. In this table there are: (1) measured serum drug and anti-drug antibody levels at the time of sample collection; and (2) estimated serum drug trough level with current dose and interval. The table on the right labeled “Estimated IFX concentration at alternate doses/intervals” provides valuable information regarding actionable treatment changes that may increase the likelihood of achieving specific therapeutic treatment targets. Organized by dose and interval this section outlines patient-specific, estimated serum drug trough levels at alternative dose and interval combinations.

### Section 3: Pharmacokinetic Drug Clearance Graph and IFX Maintenance Targets

Referring now to FIG. 18C, the “Estimated serum IFX concentration” graph is a visual depiction of the patient's unique pharmacokinetics and can serve as a useful education tool to facilitate dialog with patients about their dosing options. The “IBD maintenance IFX targets at trough” table serves as a reference of recently published studies summarizing target trough levels to increase the likelihood of achieving therapeutic outcome goals. While not prescriptive or patient specific, this table may serve as a guide when determining what IFX trough concentration may be most beneficial for the patient. In the table labeled “Estimated timepoints at which IFX concentration drops below specified targets” there are estimated time points, given in weeks following the patient's last infusion, at which drug concentrations will fall below 15, 10 and 5 μg/mL targets.

### Example 12. Clinical Decision Tool for Interleukin 17 (IL-17) Inhibitors

In this example, the clinical decision tool is developed for estimating the dose and inter-dose interval of interleukin 17 (IL-17) inhibitors, such as Secukinumab. Univariate and multivariate logistic regression analyses previously reported show that a Secukinumab level below 14 mg/L is at high risk for treatment failure. Thus, the clinical decision tool disclosed herein to reduce the dose amount or inter-dose interval of the standard while still maintaining the pre-specified threshold concentration to achieve therapeutic efficacy.

### Example 13. Clinical Decision Tool for Ixekizumab

In this example, the clinical decision tool is developed for estimating the dose and inter-dose interval of Ixekizumab. An exposure-response relationship observed in analyses previously reported show that a Ixekizumab level below 4 mcg/mL is at high risk for treatment failure. Thus, the clinical decision tool disclosed herein to reduce the dose amount or inter-dose interval of the standard while still maintaining the pre-specified threshold concentration to achieve therapeutic efficacy.

### Example 14. Clinical Decision Tool for Brodalumab

In this example, the clinical decision tool is developed for estimating the dose and inter-dose interval of Brodalumab. In some cases, the recommended dose for SKYRIZI™ (Brodalumab) is 210 mg administered by subcutaneous injection at Weeks 0, 1, and 2 followed by 210 mg every 2 weeks. The clinical decision tool disclosed herein is configured to provide guidance over dose intensification or inter-dose interval elongation while sustaining high probability that exposure is above pre-specified threshold and commensurate with high probability of treatment response. In this example the decision to elongate the dosing interval from every two weeks to every three weeks is prescribed as long as the estimates of the clinical decision tool indicate that exposure above 2.9 mg/L is sustained with 90% confidence. If an adequate response has not been achieved after 12 to 16 weeks of treatment with SKYRIZI™, treatment intensification is initiated only in the presence of concentration below pre-specified threshold, thus likely to result in greater success by bringing the levels where they were supposed to be in regard of the clinical efficacy expected for the population.

### Example 15. Clinical Decision Tool for Tildrakizumab

In this example, the clinical decision tool is developed for estimating the dose and inter-dose interval of Tildrakizumab. ILUMYA® (tildrakizumab-asmn) is an interleukin-23 antagonist indicated for the treatment of adults with moderate-to-severe plaque psoriasis who are candidates for systemic therapy or phototherapy. This biologic drug (monoclonal antibody) is administered by subcutaneous injection, comprising of 100 mg at weeks 0, 4, and every twelve weeks thereafter. The clinical decision tool disclosed herein provides a sustained with high confidence minimum effective concentration above the pre-specified threshold that minimally silence the targeted pathway.

### Example 16. Clinical Decision Tool for Guselkumab

In this example, the clinical decision tool is developed for estimating the dose and inter-dose interval of Guselkumab. TREMFYA® (Guselkumab) is an interleukin-23 blocker indicated for the treatment of adult patients with moderate-to-severe plaque psoriasis who are candidates for systemic therapy or phototherapy, and active psoriatic arthritis. For plaque psoriasis, the recommended dosage is 100 mg administered by subcutaneous injection at Week 0, Week 4 and every 8 weeks thereafter. Alternatively, in psoriatic arthritis the recommended dose is 100 mg administered by subcutaneous injection at Week 0, Week 4 and every 8 weeks thereafter. The clinical decision tool described herein can provide a recommendation to keep the concentration of TREMFYA® above minimal effective concentration associated with the plateau of response, and shorten or elongate inter-dose interval to achieve or maintain concentrations above 1 ug/mL.

### Example 17. Clinical Decision Tool for Risankizumab

In this example, the clinical decision tool is developed for estimating the dose and inter-dose interval of Risankizumab. SKYRIZI® (Risankizumab) is an interleukin-23 antagonist indicated for the treatment of moderate-to-severe plaque psoriasis in adults who are candidates for systemic therapy or phototherapy, and active psoriatic arthritis in adults. The recommended dose is 150 mg administered by subcutaneous injection at Week 0, Week 4, and every 12 weeks thereafter. The clinical decision tool described herein can provide a recommendation for doses or inter-dose intervals to achieve a desired concentration of SKYRIZI® associated with superior disease control and achievement of disease control is above 3 ug/mL with a 90% confidence to be above minimum effective concentration.

### Example 18. Clinical Decision Tool for Dupilumab

In this example, the clinical decision tool is developed for estimating the dose and inter-dose interval of Dupilumab. DUPIXIENT® (Dupilumab) binds to the alpha subunit of the interleukin-4 receptor (IL-4Ra), making it a receptor antagonist. Through blockade of IL-4Ra, dupilumab modulates signaling of both the interleukin 4 and interleukin 13 pathways. It is approved for the treatment of Atopic Dermatitis (AD) and Asthma. The clinical decision tool described here can provide a precision dosing tool to optimize Dupilumab therapy for patients with AD.

### Example 19. Clinical Decision Tool for Tralokinumab

In this example, the clinical decision tool is developed for estimating the dose and inter-dose interval of Tralokinumab. Adbry™ (Tralokinumab) targets the cytokine interleukin 13 (IL-13). The recommended dosage of Tralokinumab is an initial dose of 600 mg (four 150 mg injections), followed by 300 mg (two 150 mg injections) administered every other week. Dose of 300 mg every 4 weeks may be considered for patients below 100 kg who achieve clear or almost clear skin after 16 weeks of treatment. The clinical decision tool described here can provide a precision dosing tool to optimize Tralokinumab therapy for patients with moderate-to-severe atopic dermatitis.

### Example 20. Clinical Decision Tool for Sarilumab

In this example, the clinical decision tool is developed for estimating the dose and inter-dose interval of Sarilumab. Kevzara®(Sarilumab) is an interleukin-6 (IL-6) receptor antagonist indicated for treatment of adult patients with moderately to severely active rheumatoid arthritis who have had an inadequate response or intolerance to one or more disease-modifying antirheumatic drugs (DMARDs). It may be used as monotherapy or in combination with methotrexate (MTX) or other conventional DMARDs. The recommended dosage of KEVZARA is 200 mg once every two weeks, administered as a subcutaneous injection. The clinical decision tool described herein can provide optimum doses and inter-dose intervals of Kevzara® to maintain a desired concentration of above 10 mg/L with 90% confidence for patients with rheumatoid arthritis.

### Example 21. Inter-dose Interval Framework for Biologic Drugs in the Treatment of Psoriasis and Psoriatic Arthritis

Tables 29A-29B provide the inter-dose intervals and doses for the biologic drugs described herein for treatment of psoriasis and psoriatic arthritis generated using the clinical decision tool disclosed herein.

TABLE 29A
Inter-dose Interval Framework for Biologic Drugs in
the Treatment of Psoriasis and Psoriatic Arthritis

| | | BEST | Dose | II_{—} | II_{—} | II_{—} | II_{—} |
|---|---|---|---|---|---|---|---|
| | MOA | target | mg | intensification | normal | elongation1 | elongation2 |
| Tildrakizumab | Il-23 | 2 mg/L | 100 | 8 | 12 | 14 | 16 |
| Secukinumab | IL-17 | 14 mg/L | 300 | 2 | 4 | 6 | 8 |
| Ixekizumab | Il-17 | 4 mg/L | 80 | 2 | 4 | 6 | 8 |
| Brodalumab | IL-17 | 3 mg/L | 210 | 1 | 2 | 3 | 4 |
| Guselkumab | IL-17 | 1 mg/L | 100 | 4 | 8 | 12 | 16 |
| Risankizumab | Il-23 | 3 mg/L | 150 | 8 | 12 | 18 | 24 |
| Adalimumab | TNF | 5 mg/L | 40 | 1 | 2 | 3 | 4 |
| Ustekinumab | IL-12/23 | 5 mg/L | 45 | 8 | 12 | 14 | 16 |

II = Interdose Interval; BEST: Bayesian Estimate Sustaining Trough.
MOA = mode of action

TABLE 29B
Inter-dose Interval Framework for Biologic Drugs in the Treatment of Psoriasis and Psoriatic Arthritis

| | | | | Dose | II_{—} | II_{—} | II_{—} | II_{—} |
|---|---|---|---|---|---|---|---|---|
| | MOA | PPF1 | PPF2 | mg | intensification | normal | elongation1 | elongation2 |
| Tildrakizumab | Il-23 | <2 mg/L | >0.3 L/day | 100 | 8 | 12 | 14 | 16 |
| Secukinumab | IL-17 | <14 mg/L | >0.3 L/day | 300 | 2 | 4 | 6 | 8 |
| Ixekizumab | Il-17 | <4 mg/L | >0.3 L/day | 80 | 2 | 4 | 6 | 8 |
| Brodalumab | IL-17 | <3 mg/L | >0.3 L/day | 210 | 1 | 2 | 3 | 4 |
| Guselkumab | IL-17 | <1 mg/L | >0.3 L/day | 100 | 4 | 8 | 12 | 16 |
| Risankizumab | Il-23 | <3 mg/L | >0.3 L/day | 150 | 8 | 12 | 18 | 24 |
| Adalimumab | TNF | <5 mg/L | >0.3 L/day | 40 | 1 | 2 | 3 | 4 |
| Ustekinumab | IL-12/23 | <5 mg/L | >0.3 L/day | 45 | 8 | 12 | 14 | 16 |

II = Interdose Interval; BEST: Bayesian Estimate Sustaining Trough.
“PPF1” refers to the concentration of the biologic drug.
“PPF2” refers to the estimated clearance of the biologic drug.

### Example 22. Calculating the Optimal Cutoff for Biologic Drug Level (PPF1) and Clearance Estimates (PPF2)

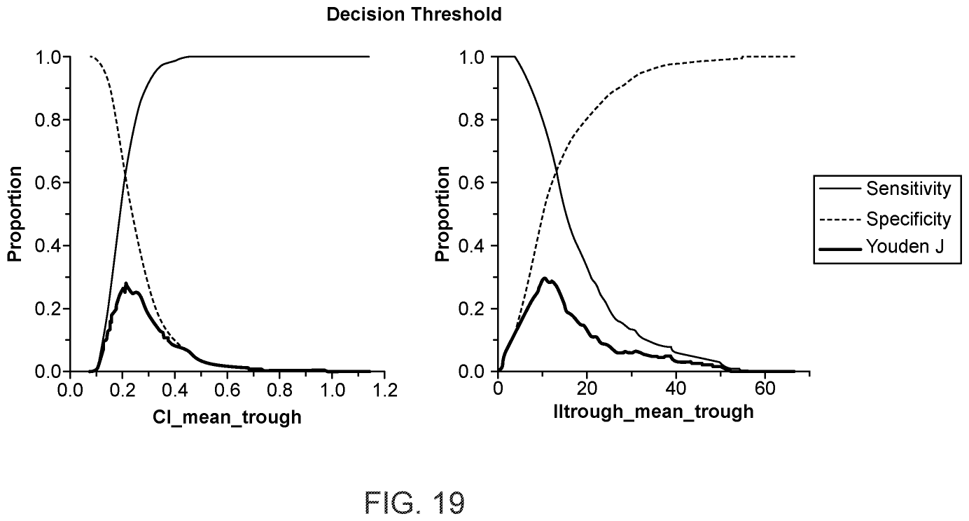

In this example, the optimal cutoff for PPF1 and PPF2 are derived from optimal Youden index that distinguished response or lack thereof, based on the clearance estimate (as seen in FIG. 19 in the left panel, with an optimal cutoff around 0.25 L/day), or the trough estimate (as seen in FIG. 19 the right panel, with an optimal cutoff around 10 mg/L). In this example the poor prognostic factors of pharmacokinetic origin consist of Clearance above 0.25 L/day either alone or in combination with low levels below 10 mg/L. Without being bound by any particular theory, a cutoff value for PPF1 and PPF2 may be derived using the optical Youden index for any biologic drug disclosed herein.

### Example 23. Infliximab Dosing Optimization for a Subject with Crohn's Disease

Use of the clinical decision tool described in Example 3 was performed again using a different set of parameter estimates provided here in Table 30.

TABLE 30
Parameter Estimates for IFX Clinical Decision Tool

| | STOCH. APPROX. | |
|---|---|---|
| VALUE | S.E. | R.S.E.(%) |

Fixed Effects

| | | | |
|---|---|---|---|
| Cl_pop | 0.28 | 0.0049 | 1.75 |
| beta_Cl_logtMedian_ALB | −1.09 | 0.048 | 4.38 |
| beta_Cl_logtMedian_ATI_QUANT | 0.22 | 0.011 | 4.90 |
| beta_Cl_logtMedian_CRP | 0.032 | 0.0031 | 9.80 |
| beta_Cl_logtMedian_WEIGHT | 0.58 | 0.037 | 6.40 |
| V1_pop | 2.11 | 0.04 | 1.89 |
| beta_V1_logtMedian_WEIGHT | 0.85 | 0.048 | 5.69 |
| Q_pop | 0.17 | 0.016 | 9.26 |
| V2_pop | 1.9 | 0.064 | 3.36 |

Standard Deviation of the Random Effects

| | | | |
|---|---|---|---|
| omega_Cl | 0.25 | 0.011 | 4.53 |
| omega_V1 | 0.25 | 0.017 | 6.95 |
| omega_Q | 1.37 | 0.08 | 5.86 |
| omega_V2 | 0.48 | 0.03 | 6.23 |
| gamma_Cl | 0.18 | 0.0039 | 2.17 |
| gamma_V1 | 0.24 | 0.0068 | 2.80 |

Error Model Parameters

| | | | |
|---|---|---|---|
| b | 0.13 | 0.004 | 3.02 |

### Example 24: Poor Prognostic Factors Of Pharmacokinetic Origin (PPFPK) in Treating Autoimmune (AI) Patients

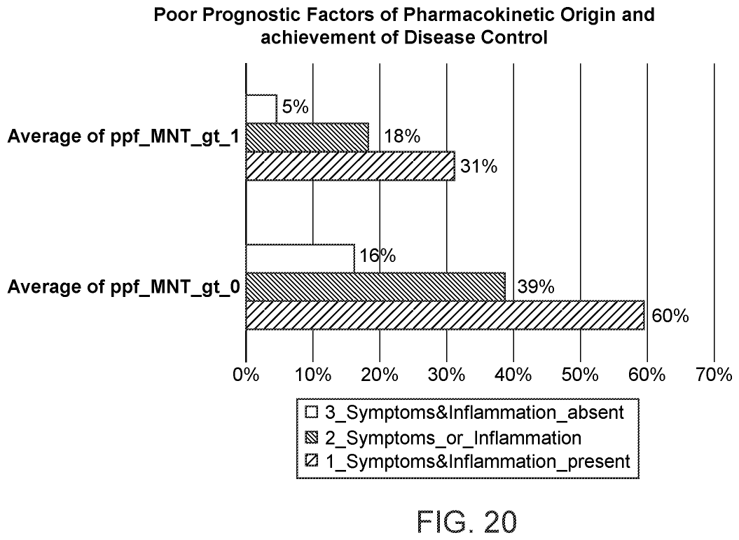

Poor prognostic factor of pharmacokinetic origin (PPFPK) in treating autoimmune (AI) patients was investigated. The clinical decision tool described herein utilized a Bayesian data assimilation. The PPFPK included 1) an intrinsic property of clearance above 0.294 L/day and 2) Infliximab (IFX) levels at the inter-dose interval below 5 mg/L were used either alone or in combination. FIG. 20 shows the percent of AI patient with gastrointestinal disease that have adequate disease control in the presence of one or both PPFPK (e.g., ppf_MNT_gt_1 and ppf_MNT_gt_0). As shown in FIG. 20, only 5% of AI patient with gastrointestinal disease that have both PPFPK (“ppf_MNT_gt_1”) presented with adequate disease control (e.g., both symptoms and inflammation absent). For these patients with poor disease control, the monoclonal antibody was stopped and a small molecule targeted disease modifier was initiated (e.g., JAK kinase inhibitors, such as Upadacitinib or Sphingosine 1-P receptor modulator, such as ozanimod).

In this example, across multiple studies, the poor clinical status resulting in part from the ineffective achievement of exposure commensurate with adequate disease control is illustrated. Target patients presenting with PPFPK were treated through dose intensification to address poor exposure associated with intrinsic clearance. This poor exposure was an irremediable intrinsic property of the AI patient.

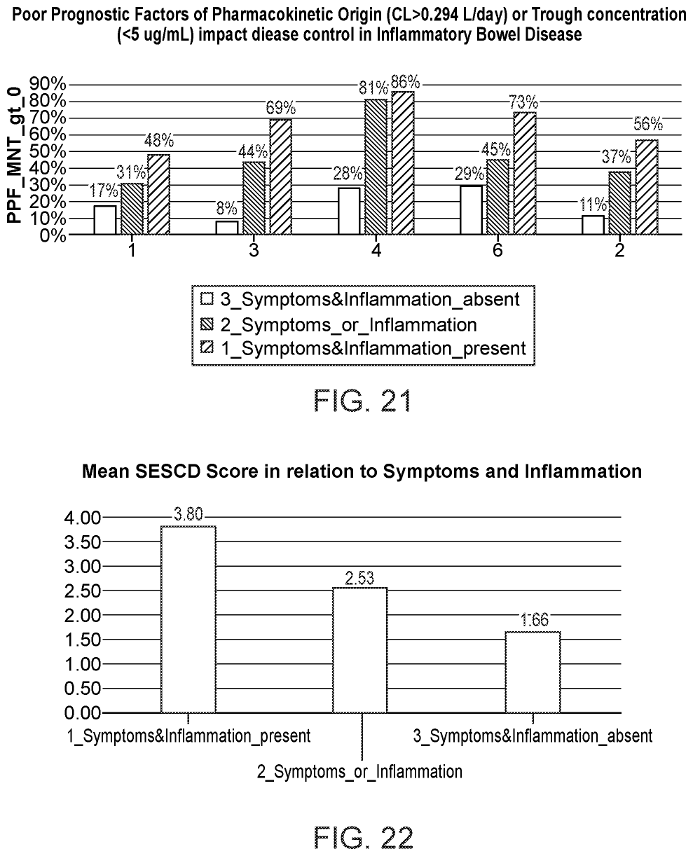

FIG. 21 shows the PPFPK impact on disease control in IBD in patients from five (5) different studies with one PPF (“ppf_MNT_gt_0”). The patients had either a clearance >0.294 L/day or a trough IFX concentration of less than 5 ug/mL.

In the presence of both PPFPK and inadequate disease control achieved during maintenance, target patients received the following treatment: 1) IFX dose was increased if the PPF associated with low inter-dose interval levels (<5 mg/L) could be eliminated by obtaining high probability of IFX levels greater than 5 mg/L; or 2) IFX was stopped and a small molecule therapy (e.g., inhibitor of JAK, S1P) was initiated in the presence of both PPFPK that could not be addressed by dose intensification.

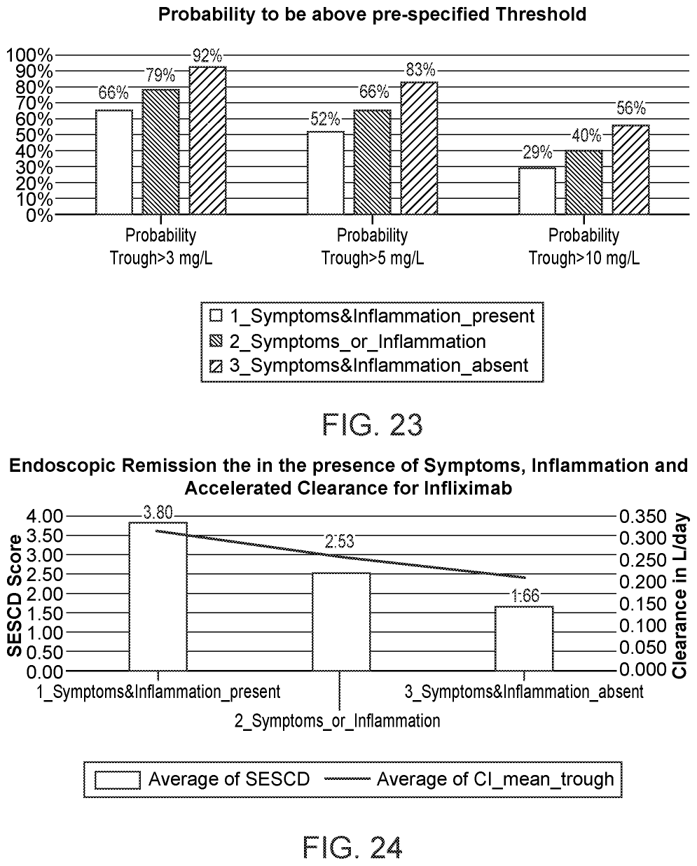

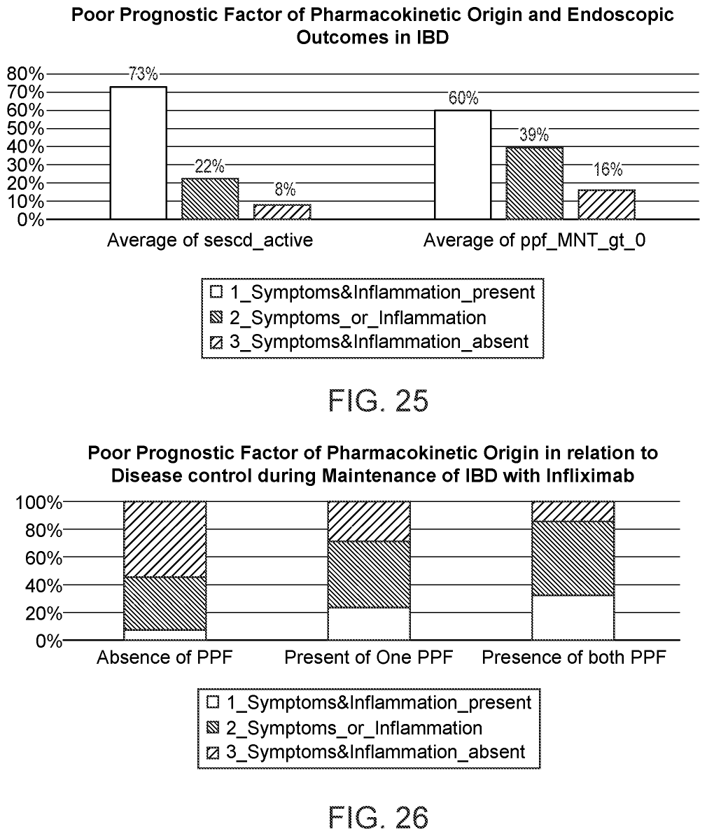

FIG. 22 provides the association between clinical status in the presence or absence of inflammation and symptoms, and endoscopic outcome (“simple endoscopic score for Crohn's disease (SESCD)”). As shown, superior endoscopic outcome was achieved in the absence of inflammation and symptoms. Additionally, FIG. 23 illustrates the superior clinical outcome where disease remission was achieved in the absence of inflammation. The absence of inflammation was associated with an increased probability to achieve exposure above pre-specified threshold. The superior clinical outcome was also associated with higher inter-dose interval concentration and lower clearance, as shown in FIG. 24 and Table 31. In Table 31, SESCD refers to simple endoscopic score for Crohn's disease, Cl_mean_trough refers to trough of mean clearance levels, and Iltrough_mean_trough refers trough of mean IFX levels. Additionally, FIG. 25 illustrates that the presence of inflammation and symptoms are associated with patients with an active Simple endoscopic score for Crohn's disease (SES-CD) or have a PPFPK.

TABLE 31
Endoscopic remission in the presence of symptoms,
inflammation and accelerated clearance for IFX.

| | Average | Average of | Average of |
|---|---|---|---|
| | SESCD | Cl_mean_trough | Iltrough_mean_trough |
| Symptoms and | 3.80 | 0.316 | 7.7 |
| inflammation | | | |
| present | | | |
| Symptoms or | 2.53 | 0.257 | 10.4 |
| inflammation | | | |
| present | | | |
| Symptoms and | 1.66 | 0.209 | 13.8 |
| inflammation | | | |
| absent | | | |

Finally, FIG. 26 and Table 32 provide a summary of the PPFPK in treating AI patients. FIG. 26 provides the PPFPK in relation to disease control during maintenance of IBD with IFX. In the absence of both PPF, it was likely the symptoms and inflammation were absence. In the present of one PPF it was likely the symptoms or inflammation was present. Finally, in the presents of both PPF, it was likelihood that the symptoms and inflammation were present further increased.

TABLE 32
PPFPK in relation to disease control
during maintenance of IBD with IFX

| | Symptoms and | Symptoms or | Symptoms and |
|---|---|---|---|
| | inflammation | inflammation | inflammation |
| | present | present | absent |
| Absence of PPF | 84 | 345 | 493 |
| Presence of one PPF | 59 | 116 | 69 |
| Presence of both PPF | 65 | 103 | 27 |
| Total | 208 | 564 | 589 |

While preferred embodiments of the present subject matter have been shown and described herein, it will be obvious to those skilled in the art that such embodiments are provided by way of example only. Numerous variations, changes, and substitutions will now occur to those skilled in the art without departing from the present subject matter. It should be understood that various alternatives to the embodiments of the present subject matter described herein may be employed in practicing the present subject matter.

What is claimed is:

1. A method for treating an immune mediated inflammatory disease in a subject, the method comprising:
(a) analyzing a biological sample obtained from a subject with an immune-mediated inflammatory disease, wherein the analyzing comprises:
(i) obtaining or having obtained the biological sample from the subject; and
(ii) quantifying or having quantified analytes in the biological sample, wherein the analytes comprise: (1) a level of a biologic drug, (2) a level of autoantibodies against the biologic drug, and (3) a level of albumin, wherein the subject has received a treatment for the immune-mediated inflammatory disease that comprises a current dose of the biologic drug administered to the subject at a current inter-dose interval;
(b) determining a likelihood of achieving a pre-specified threshold concentration of the biologic drug in the subject based, at least in part, on (1) the level of the biologic drug, the level of the autoantibodies, and the level of albumin quantified in (a)(ii), and (2) the current dose of the biologic drug and the current inter-dose interval; and
(c) if the likelihood of achieving the pre-specified threshold concentration of the biologic drug is above 50%, then:
(1) administering the current dose of the biologic drug to the subject at the current inter-dose interval; or
(2) administering a dose of the biologic drug that is (i) lower than the current dose to the subject at the current inter-dose interval, (ii) the same as the current dose to the subject at an inter-dose interval that is longer than the current inter-dose interval, or (iii) lower than the current dose to the subject at an inter-dose interval that is longer than the current inter-dose interval;
(d) if the likelihood of achieving the pre-specified threshold concentration of the biologic drug is below 50%, then administering a dose of the biologic drug that is (i) higher than the current dose to the subject in the current inter-dose interval, (ii) the current dose at an inter-dose interval that is shorter than the current inter-dose interval, or (iii) higher than the current dose to the subject in the inter-dose interval that is shorter than the current inter-dose interval; or
(e) if the dose of the biologic drug in (d) is above a maximum dose and the inter-dose interval in (d) is less than or equal to a minimum inter-dose interval, then discontinuing the treatment comprising the biologic drug.

2. The method of claim 1, wherein the biologic drug comprises an antibody or antigen-binding fragment thereof.

3. The method of claim 2, wherein the antibody comprises a monoclonal antibody.

4. The method of any one of claims 1-3, the biologic drug comprises adalimumab (ADA).

5. The method of claim 4, wherein the current dose of the biologic drug is 40 milligrams (mg), and the current inter-dose interval is every two weeks.

6. The method of claim 4, wherein the dose of the biologic drug in (c) is 20 to 80 mg and the inter-dose interval in (c) is every week to every six weeks.

7. The method of claim 4, wherein the maximum dose in (e) is 80 mg, and the minimum inter-dose interval is weekly.

8. The method of any one of claims 1-7, wherein the biologic drug comprises infliximab (IFX).

9. The method of claim 8, wherein the current dose of the biologic drug is about 5 milligrams per kilogram (mg/kg), and the current inter-dose interval is every eight weeks.

10. The method of claim 8, wherein the dose of the biologic drug in (c) is about 3 to 15 mg/kg and the inter-dose interval in (c) is every four to twelve weeks.

11. The method of claim 8, wherein the maximum dose in (e) is 15 mg/kg, and the minimum inter-dose interval is every four weeks.

12. The method of any one of claims 1-11, wherein the biologic drug comprises tocilizumab (TCZ).

13. The method of claim 12, wherein the current dose of the biologic drug is 162 milligrams (mg), and the current inter-dose interval is every two weeks.

14. The method of claim 12, wherein the dose of the biologic drug in (c) is 162 mg and the inter-dose interval in (c) is twice a week to every six weeks.

15. The method of claim 12, wherein the maximum dose in (e) is 162 mg, and the minimum inter-dose interval is twice a week.

16. The method of any one of claims 1-15, wherein the predetermined threshold concentration of the biologic drug comprises between about 1 mg/L and 10 mg/L.

17. The method of claim 16, wherein the predetermined threshold concentration of the biologic drug comprises about 5 mg/L to about 10 mg/L when the biologic drug is ADA.

18. The method of claim 16, wherein the predetermined threshold concentration of the biologic drug comprises about 5 mg/L to about 10 mg/L when the biologic drug is IFX.

19. The method of claim 16, wherein the predetermined threshold concentration of the biologic drug comprises about 1 mg/L to about 7.5 mg/L when the biologic drug is TCZ.

20. The method of any one of claims 1-19, wherein the determining the likelihood of achieving the pre-specified threshold concentration of the biologic drug in the subject is further based, at least in part, on a weight of the subject.

21. The method of claim 20, wherein the determining the likelihood of achieving the pre-specified threshold concentration of the biologic drug in the subject comprises estimating a clearance rate of the biologic drug in the subject based, at least in part, on the weight of the subject and the level of albumin quantified in (a)(ii).

22. The method of claim 21, wherein the determining the likelihood of achieving the pre-specified threshold concentration of the biologic drug in the subject further comprises determining whether the subject has a poor prognostic factor of pharmacokinetic origin (PPFPK), wherein the PPFPK is determined based, at least in part, on the level of the biologic drug quantified in (a)(ii) and the clearance rate.

23. The method of claim 22, wherein, if the treatment comprising the biologic drug is discontinued in (e), then administering to the subject a small molecule inhibitor of a Janus Kinase (JAK) or a sphingosine 1-phosphate (S1P) receptor modulator.

24. The method of claim 23, wherein:
(a) the small molecule inhibitor of JAK comprises baricitinib, tofacitinib, or upadacitinib, or any combination thereof; and
(b) wherein the S1P receptor modulator comprises fingolimod, siponimod, ozanimod, or ponesimod, or any combination thereof.

25. The method of any one of claims 1-24, wherein, if the treatment comprising the biologic drug is discontinued in (e), then administering to the subject another biologic drug that differs from the biologic drug.

26. The method of any one of claims 1-25, wherein the determining the likelihood of achieving the pre-specified threshold concentration of the biologic drug in the subject comprises applying a algorithm to the analytes quantified in (a)(ii).

27. The method of claim 26, wherein the algorithm comprises a Naive Bayes classifier algorithm.

28. The method of claim 26, wherein the algorithm comprises a Metropolis Hastings algorithm.

29. The method of any one of claims 26-28, wherein the determining the likelihood of achieving the pre-specified threshold concentration of the biologic drug in the subject in (b) comprises utilizing a model comprising:
(i) establishing a first set of parameter estimates from a reference population, wherein the reference population has received the biologic drug for treatment of the immune-mediated inflammatory disease;
(ii) deriving a second set of parameter estimates for the model based at least in part on the first set of parameter estimates established in (i); and
(iii) inputting data comprising (i) the analytes quantified in the biological sample obtained from the subject into the model and (ii) the current dose of the biologic drug and the current inter-dose interval; and
(iv) interrogating the model based at least in part based on the data, wherein the subject is not a part of the reference population.

30. The method of claim 29, wherein the data further comprises a level of a level of C-Reactive Protein (CRP).

31. The method of claim 29 or 30, wherein the data further comprises a level of interleukin 6 (IL-6).

32. The method of any one of claims 29-31, wherein the data further comprises a weight of the subject.

33. The method of any one of claims 29-32, wherein the data further comprises a body mass index (BMI) of the subject.

34. The method of any one of claims 26-33, wherein the determining the likelihood of achieving the pre-specified threshold concentration of the biologic drug in the subject is further based, at least in part, on a weight of the subject.

35. The method of claim 34, wherein the biological sample comprises a serum sample.

36. The method of claim 34 or 35, wherein the likelihood that high is equal to or about 90%.

37. The method of any one of claims 34-36, wherein the analytes quantified in (a)(ii) further comprise a level of C-Reactive Protein (CRP).

38. The method of any one of claims 34-37, wherein the analytes quantified in (a)(ii) further comprise interleukin 6 (IL-6).

39. The method of any one of claims 34-38, wherein the analytes quantified in (a)(ii) are quantified with an assay comprising a mobility shift assay or a solid-phase immunoassay.

40. The method of claim 39, wherein the solid-phase immunoassay comprises an enzyme-linked immunoassay (ELISA).

41. The method of any one of claims 34-40, further comprising receiving information about the subject, wherein the information comprises a severity of the immune-mediated inflammatory disease or a symptom thereof.

42. The method of claim 41, wherein the severity of the immune-mediated inflammatory disease comprises a disease remission, a disease recurrence, a disease type, or any combination thereof.

43. The method of claim 41 or [00198], wherein the severity of the symptom of the immune-mediated inflammatory disease comprises a frequency of the symptom, a type of the symptom, or a combination thereof.

44. The method of any one of claims 41-[00198], wherein the severity of the immune-mediated inflammatory disease or symptom thereof is based on a clinical disease activity index (CDAI) score.

45. The method of any one of claims 41-[00198], wherein the receiving the information about the subject comprises receiving one or more electronic medical records (EMRs), wherein the one or more EMRs comprise the information.

46. The method of any one of claims 41-[00198], wherein the information is self-reported by the subject.

47. The method of claim 46, wherein the information is self-reported by the subject inputting the information into a mobile application on a personal electronic device of the subject.

48. The method of any one of claims 1-47, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

49. The method of claim 48, wherein the IBD comprises Crohn's disease (CD).

50. The method of claim 48, wherein the IBD comprises ulcerative colitis (UC).

51. The method of any one of claims 1-50, wherein the subject has received the treatment comprising the current dose of the biologic drug administered to the subject at the current inter-dose interval for at least 14 contiguous weeks.

52. The method of any one of claims 1-51, wherein the subject has received the treatment regimen comprising the current dose of the biologic drug administered to the subject at the current inter-dose interval at least once.

53. The method of any one of claims 1-52, wherein the biological sample is obtained in (a)(i) up to 20 days following a last administration of the biologic drug at the current dose.

54. A method for achieving a threshold biologic drug concentration value in a subject, the method comprising:
(a) initializing a model of a biologic drug concentration profile for a biologic drug, wherein the model comprises data received from a reference population that were or currently are being treated with the biologic drug for treatment of an immune-mediated inflammatory disease;
(b) generating subject specific parameters relating to pharmacokinetic performance of the biologic drug in the subject;
(c) simulating the biologic drug concentration profile for the subject based on the subject specific parameters and the data from the reference population;
(d) updating the model based on newly received data from the subject in (b) and newly received data from the reference population in (a); and
(e) estimating a dose of the biologic drug at an inter-dose interval to achieve the threshold biologic drug concentration value in the subject with the model updated in (d), wherein the threshold biologic drug concentration is sufficient to treat the immune-mediated inflammatory disease in the subject.

55. The method of claim 54, wherein generating the subject specific parameters comprises compiling information about the subject.

56. The method of claim 55, wherein the information about the subject comprises a severity of the immune-mediated inflammatory disease or a symptom thereof.

57. The method of claim 56, wherein the data is received from the subject via a mobile application on a personal electronic device of the subject.

58. The method of claim 56 or 57, wherein the severity of the immune-mediated inflammatory disease comprises a disease remission, a disease recurrence, a disease type, or any combination thereof.

59. The method of any one of claims 56-58, wherein the severity of the symptom of the immune-mediated inflammatory disease comprises a frequency of the symptom, a type of the symptom, or a combination thereof.

60. The method of any one of claims 56-59, wherein the severity of the immune-mediated inflammatory disease or symptom thereof is based on a clinical disease activity index (CDAI) score.

61. The method of any one of claims 55-60, wherein the information about the subject comprises a weight or body mass index (BMI) of the subject.

62. The method of any one of claims 55-61, wherein the data is contained in one or more electronic medical records (EMRs).

63. The method of any one of claims 54-62, wherein the subject specific parameters comprise two or more of:
(a) a clearance (C);
(b) a volume of distribution of a central compartment (Vc);
(c) intercompartmental clearance;
(d) a volume of a peripheral compartment (Vp);
(e) absorption rate constant;
(f) maximum velocity at high biologic drug concentrations (Vmax);
(g) affinity of the biologic drug to a substrate;
(h) proportional error;
(i) body weight; or
(j) log transformed covariates on the subject specific parameters in one or more of (a) to (g) as determined using non-linear mixed effect modeling; or
(k) any combination thereof.

64. The method of any one of claims 54-63, wherein the data received from the reference population comprises:
(a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5);
(b) a weight of individuals in the reference population;
(c) a body mass index (BMI) of the individuals in the reference population; or
(d) any combination of (a) to (c).

65. The method of any one of claims 54-64, wherein the newly received data from the subject comprises:
(a) a level of one or more analytes in a biological sample obtained from the subject, wherein the one or more analytes comprises (1) a biologic drug, (2) autoantibodies against the biologic drug, (3) albumin, (4) IL-6, (5) CRP, or (6) any combination of (1) to (5);
(b) a weight of the subject;
(c) BMI of the subject; or
(d) any combination of (a) to (c).

66. The method of any one of claims 54-65, wherein estimating the dose of the biologic drug at the inter-dose interval to achieve the threshold biologic drug concentration value in the subject comprises estimating a clearance rate of the biologic drug in the subject based, at least in part, on the weight of the subject and the level of albumin in the biological sample obtained from the subject in the newly received data from the subject.

67. The method of claim 66, wherein estimating the dose of the biologic drug at the inter-dose interval to achieve the threshold biologic drug concentration value in the subject further comprises determining whether the subject has a poor prognostic factor of pharmacokinetic origin (PPFPK), wherein the PPFPK is determined based, at least in part, on the level of the biologic drug in (a)(1) and the clearance rate.

68. The method of any one of claims 65-67, wherein the level of one or more analytes in a biological sample obtained from the subject is measured using a mobility shift assay or a solid-phase immunoassay.

69. The method of claim 68, wherein the solid-phase immunoassay comprises an enzyme-linked immunoassay (ELISA).

70. The method of claim 65, wherein the biological sample comprises a serum sample.

71. The method of any one of claims 65-70, wherein the biological sample is obtained from the subject up to 20 days following a last administration of the biologic drug to the subject.

72. The method of any one of claims 54-71, wherein the model comprises a trained model.

73. The method of claim 72, wherein the model comprises a Bayesian assimilation.

74. The method of claim 72, wherein the model comprises a non-linear mixed effects model (NLME).

75. The method of claim 72, wherein the model comprises a Markov Chain Monte Carlo (MCMC) simulation.

76. The method of any one of claims 54-75, wherein estimating the dose of the biologic drug at the inter-dose interval is performed with greater than a 50% confidence.

77. The method of any one of claims 54-75, wherein estimating the dose of the biologic drug at the inter-dose interval is performed with between about 50% and 90% confidence.

78. The method of any one of claims 54-75, wherein estimating the dose of the biologic drug at the inter-dose interval is performed with greater than or equal to about a 90% confidence.

79. The method of any one of claims 54-78, wherein the biologic drug comprises an antibody or antigen-binding fragment thereof.

80. The method of claim 79, wherein the antibody comprises a monoclonal antibody.

81. The method of any one of claims 54-78, wherein the biologic drug comprises adalimumab (ADA).

82. The method of claim 81, wherein the dose of the ADA is 40 mg and the inter-dose interval is every two weeks.

83. The method of claim 81, wherein the dose of the ADA is less than or equal to about 80 mg and the inter-dose interval is greater than or equal to about every week.

84. The method of any one of claims 54-78, wherein the biologic drug comprises infliximab (IFX).

85. The method of claim 84, wherein the dose of the IFX is 5 mg/kg and the inter-dose interval is every eight weeks.

86. The method of claim 84, wherein the dose of the IFX is less than or equal to about 15 mg/kg an d the inter-dose interval is greater than or equal to about four weeks.

87. The method of any one of claims 54-78, wherein the biologic drug comprises tocilizumab (TCZ).

88. The method of claim 87, wherein the dose of the TCZ is 162 mg and the inter-dose interval is every two weeks.

89. The method of claim 87, wherein the dose of the TCZ is less than or equal to about 162 mg and the inter-dose interval is greater than or equal to about twice a week.

90. The method of any one of claims 54-89, wherein the threshold biologic drug concentration value comprises between about 1 mg/L and 10 mg/L.

91. The method of claim 90, wherein the threshold biologic drug concentration value comprises about 5 to 10 mg/L when the biologic drug is ADA.

92. The method of claim 90, wherein the threshold biologic drug concentration value comprises about 5 to 10 mg/L when the biologic drug is IFX.

93. The method of claim 90, wherein the threshold biologic drug concentration value comprises about 1 to 7.5 mg/L when the biologic drug is TCZ.

94. The method of any one of claims 54-93, further comprising providing a recommendation to discontinue treatment of the immune-mediated inflammatory disease with the biologic drug if the dose of the biologic drug is above a maximum dose amount.

95. The method of claim 94, wherein the maximum dose amount is 80 mg when the biologic drug comprises ADA.

96. The method of claim 94, wherein the maximum dose amount is 15 mg/kg when the biologic drug comprises IFX.

97. The method of claim 94, wherein the maximum dose amount is 162 mg when the biologic drug comprises TCZ.

98. The method of claim 94, wherein the recommendation further comprises a treatment regimen comprising a small molecule inhibitor of a Janus Kinase (JAK) or a sphingosine 1-phosphate (S1P) receptor modulator.

99. The method of claim 98, wherein:
(a) the small molecule inhibitor of JAK comprises baricitinib, tofacitinib, or upadacitinib, or any combination thereof; and
(b) wherein the S1P receptor modulator comprises fingolimod, siponimod, ozanimod, or ponesimod, or any combination thereof.

100. The method of any one of claims 54-99, wherein the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

101. The method of claim 100, wherein the IBD comprises Crohn's disease (CD).

102. The method of claim 100, wherein the IBD comprises ulcerative colitis (UC).

103. The method of any one of claims 54-102, wherein the subject has received a treatment comprising a current dose of the biologic drug administered to the subject at a current inter-dose interval for at least 14 contiguous weeks.

104. The method of any one of claims 54-103, wherein the subject has received a treatment regimen comprising a current dose of the biologic drug administered to the subject at a current inter-dose interval at least once.

105. A computer-implemented method of training an algorithm that determines an biologic drug profile of a biologic drug for a subject having an immune-mediated inflammatory disease, the method comprising:
(a) receiving data from a database, wherein the data is related to a pharmacokinetic performance of the biologic drug in individuals from a reference population having the immune-mediated inflammatory disease that have been treated with the biologic drug;
(b) establishing a first set of parameter estimates from the data;
(c) deriving a second set of parameter estimates for a model based at least in part on the first set of parameter estimates;
(d) receiving subject specific data related to the pharmacokinetic performance of the biologic drug in the subject;
(e) updating the model based at least in part on the subject specific data received in (d); and
(f) determining a biologic drug profile for the subject with the model, wherein the biologic drug profile comprises a dose of the biologic drug at an inter-dose interval estimated to achieve a threshold biologic drug concentration value in the subject that is sufficient to treat the immune-mediated inflammatory disease in the subject.

106. The method of claim 105, wherein the subject specific data is received from the subject by inputting the data into a mobile application on the subject's personal electronic device.

107. The method of claim 105 or 106, wherein the subject specific data comprises:
(a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5), measured in a biological sample obtained from the subject;
(b) a weight of the subject;
(c) a body mass index (BMI) of the subject; or
(d) any combination of (a) to (c).

108. The method of any one of claims 105-107, wherein the subject specific data comprises information comprising a severity of the immune-mediated inflammatory disease or a symptom thereof.

109. The method of claim 108, wherein the severity of the immune-mediated inflammatory disease comprises a disease remission, a disease recurrence, a disease type, or any combination thereof.

110. The method of claim 108 or 109, wherein the severity of the symptom of the immune-mediated inflammatory disease comprises a frequency of the symptom, a type of the symptom, or a combination thereof.

111. The method of any one of claims 108-110, wherein the severity of the immune-mediated inflammatory disease or symptom thereof is based on a clinical disease activity index (CDAI) score.

112. The method of any one of claims 108-111, wherein the information about the subject comprises a weight or body mass index (BMI) of the subject.

113. The method of any one of claims 105-112, wherein the subject specific data is contained in one or more electronic medical records (EMRs).

114. The method of any one of claims 105-113, wherein the data comprises:
(a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5);
(b) a weight of individuals in the reference population;
(c) a body mass index (BMI) of the individuals in the reference population; or
(d) any combination of (a) to (c).

115. The method of any one of claims 105-114, wherein the dose of the biologic drug at the inter-dose interval is determined by estimating a clearance rate of the biologic drug in the subject based, at least in part, on the weight of the subject and the level of albumin in a biological sample of the subject.

116. The method of any one of claims 105-115, wherein the dose of the biologic drug at the inter-dose interval is determined by:
(i) estimating a clearance rate of the biologic drug in the subject based, at least in part, on the weight of the subject and the level of albumin in a biological sample of the subject; and
(ii) determining whether the subject has a poor prognostic factor of pharmacokinetic origin (PPFPK), wherein the PPFPK is determined based, at least in part, on a level of the biologic drug in the biological sample of the subject and the clearance rate.

117. The method of any one of claims 105-116, wherein the data comprises information comprising a severity of the immune-mediated inflammatory disease or a symptom thereof.

118. The method of claim 117, wherein the severity of the immune-mediated inflammatory disease comprises a disease remission, a disease recurrence, a disease type, or any combination thereof.

119. The method of claim 117 or 118, wherein the severity of the symptom of the immune-mediated inflammatory disease comprises a frequency of the symptom, a type of the symptom, or a combination thereof.

120. The method of any one of claims 117-119, wherein the severity of the immune-mediated inflammatory disease or symptom thereof is based on a clinical disease activity index (CDAI) score.

121. The method of any one of claims 117-120, wherein the information about the subject comprises a weight or body mass index (BMI) of the subject.

122. The method of any one of claims 117-121, wherein the clinical laboratory data is contained in one or more electronic medical records (EMRs).

123. The method of any one of claims 105-122, wherein the first set of parameter estimates comprises:
(a) a clearance (C);
(b) a volume of distribution of a central compartment (Vc);
(c) intercompartmental clearance;
(d) a volume of a peripheral compartment (Vp);
(e) absorption rate constant;
(f) maximum velocity at high biologic drug concentrations (Vmax);
(g) affinity of the biologic drug to a substrate;
(h) proportional error;
(i) body weight; or
(j) log transformed covariates on the subject specific parameters in one or more of (a) to (g) as determined using non-linear mixed effect modeling; or
(k) any combination thereof.

124. The method of any one of claims 105-122, wherein the second set of parameter estimates comprises:
(a) a clearance (C);
(b) a volume of distribution of a central compartment (Vc);
(c) intercompartmental clearance;
(d) a volume of a peripheral compartment (Vp);
(e) absorption rate constant;
(f) maximum velocity at high biologic drug concentrations (Vmax);
(g) affinity of the biologic drug to a substrate;
(h) proportional error;
(i) body weight; or
(j) log transformed covariates on the subject specific parameters in one or more of (a) to (g) as determined using non-linear mixed effect modeling; or
(k) any combination thereof.

125. The method of any one of claims 105-123, wherein the algorithm comprises a Naive Bayes classifier algorithm.

126. The method of any one of claims 105-123, wherein the algorithm comprises anon-linear mixed effects model (NLME).

127. The method of any one of claims 105-123, wherein the algorithm comprises a Metropolis Hastings algorithm.

128. The method of any one of claims 105-127, wherein the dose and inter-dose interval are estimated to achieve the threshold biologic drug concentration value for the subject with a probability that is greater than a 50%.

129. The method of any one of claims 105-127, wherein the dose and inter-dose interval are estimated to achieve the threshold biologic drug concentration value for the subject with a probability that is between about 50% and 90%.

130. The method of any one of claims 105-127, wherein the dose and inter-dose interval are estimated to achieve the threshold biologic drug concentration value for the subject with a probability that is greater than or equal to about a 90%.

131. The method of any one of claims 105-130, wherein the biologic drug comprises an antibody or antigen-binding fragment thereof.

132. The method of claim 131, wherein the antibody comprises a monoclonal antibody.

133. The method of any one of claims 105-130, wherein the biologic drug comprises adalimumab (ADA).

134. The method of claim 133, wherein the dose of the ADA is 40 mg and the inter-dose interval is every two weeks.

135. The method of claim 133, wherein the dose of the ADA is less than or equal to about 80 mg and the inter-dose interval is greater than or equal to about every week.

136. The method of any one of claims 105-130, wherein the biologic drug comprises infliximab (IFX).

137. The method of claim 136, wherein the dose of the IFX is 5 mg/kg and the inter-dose interval is every eight weeks.

138. The method of claim 136, wherein the dose of the IFX is less than or equal to about 15 mg/kg and the inter-dose interval is greater than or equal to about every four weeks.

139. The method of any one of claims 105-130, wherein the biologic drug comprises tocilizumab (TCZ).

140. The method of claim 139, wherein the dose of the TCZ is 162 mg and the inter-dose interval is every two weeks.

141. The method of claim 139, wherein the dose of the TCZ is less than or equal to about 162 mg and the inter-dose interval is greater than or equal to about twice every week.

142. The method of any one of claims 105-141, wherein the threshold biologic drug concentration value comprises between about 1 mg/L and 10 mg/L.

143. The method of claim 142, wherein the threshold biologic drug concentration value comprises about 5 to 10 mg/L when the biologic drug is ADA.

144. The method of claim 142, wherein the threshold biologic drug concentration value comprises about 5 to 10 mg/L when the biologic drug is IFX.

145. The method of claim 142, wherein the threshold biologic drug concentration value comprises about 1 to 7.5 mg/L when the biologic drug is TCZ.

146. The method of any one of claims 105-145, further comprising providing a recommendation to discontinue treatment of the immune-mediated inflammatory disease with the biologic drug if the dose of the biologic drug is above a maximum dose amount.

147. The method of claim 146, wherein the maximum dose amount is 80 mg when the biologic drug comprises ADA.

148. The method of claim 146, wherein the maximum dose amount is 15 mg/kg when the biologic drug comprises IFX.

149. The method of claim 146, wherein the maximum dose amount is 162 mg when the biologic drug comprises TCZ.

150. The method of claim 146, wherein the recommendation further comprises a treatment regimen comprising a small molecule inhibitor or a Janus Kinase (JAK) or a sphingosine 1-phosphate (S1P) receptor modulator.

151. The method of claim 150, wherein:
(a) the small molecule inhibitor of JAK comprises baricitinib, tofacitinib, or upadacitinib, or any combination thereof; and
(b) wherein the SIP receptor modulator comprises fingolimod, siponimod, ozanimod, or ponesimod, or any combination thereof.

152. The method of any one of claims 105-151, wherein the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

153. The method of claim 152, wherein the IBD comprises Crohn's disease (CD).

154. The method of claim 152, wherein the IBD comprises ulcerative colitis (UC).

155. The method of any one of claims 105-154, wherein the subject has received a treatment comprising a current dose of the biologic drug administered to the subject at a current inter-dose interval for at least 14 contiguous weeks.

156. The method of any one of claims 105-154, wherein the subject has received a treatment regimen comprising a current dose of the biologic drug administered to the subject at a current inter-dose interval at least once.

157. A computer-implemented system for achieving a threshold biologic drug concentration value in a subject, the computer-implemented system comprising:
a computing device comprising at least one processor;
an operating system configured to perform executable instructions;
a memory; and
a computer program including instructions executable by the computing device to create an application comprising:
a software module configured to initialize a model of a biologic drug concentration profile for a biologic drug, wherein the model comprises data related to a pharmacokinetic performance of the biologic drug in individuals from a reference population having the immune-mediated inflammatory disease that have been treated with the biologic drug;
a software module configured to establish a first set of parameter estimates from the data;
a software module configured to derive a second set of parameter estimates for a model based at least in part on the first set of parameter estimates;
a software module configured to receive subject specific data related to the pharmacokinetic performance of the biologic drug in the subject; and
a software module configured to update the model based at least in part on the subject specific data; and
a software module configured to determine a biologic drug profile for the subject, wherein the biologic drug profile comprises a dose of the biologic drug at an inter-dose interval estimated to achieve a threshold biologic drug concentration value in the subject that is sufficient to treat the immune-mediated inflammatory disease in the subject.

158. The system of claim 157, wherein the subject specific parameters comprise information about the subject.

159. The system of claim 157 or 158, wherein the data is received from the subject via a mobile application on a personal electronic device of the subject.

160. The system of claim 158, wherein the information about the subject comprises a severity of the immune-mediated inflammatory disease or a symptom thereof.

161. The system of claim 160, wherein the severity of the immune-mediated inflammatory disease comprises a disease remission, a disease recurrence, a disease type, or any combination thereof.

162. The system of claim 160 or 161, wherein the severity of the symptom of the immune-mediated inflammatory disease comprises a frequency of the symptom, a type of the symptom, or a combination thereof.

163. The system of any one of claims 160-162, wherein the severity of the immune-mediated inflammatory disease or symptom thereof is based on a clinical disease activity index (CDAI) score.

164. The system of any one of claims 158-163, wherein the information about the subject comprises a weight or body mass index (BMI) of the subject.

165. The system of any one of claims 157-164, wherein the data is contained in one or more electronic medical records (EMRs).

166. The system of any one of claims 157-165, wherein the subject specific parameters comprise two or more of:
(a) a clearance (C);
(b) a volume of distribution of a central compartment (Vc);
(c) intercompartmental clearance;
(d) a volume of a peripheral compartment (Vp);
(e) absorption rate constant;
(f) maximum velocity at high biologic drug concentrations (Vmax);
(g) affinity of the biologic drug to a substrate;
(h) proportional error;
(i) body weight; or
(j) log transformed covariates on the subject specific parameters in one or more of (a) to (g) as determined using non-linear mixed effect modeling; or
(k) any combination thereof.

167. The system of any one of claims 157-166, wherein the data received from the reference population comprises:
(a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5);
(b) a weight of individuals in the reference population;
(c) a body mass index (BMI) of the individuals in the reference population; or
(d) any combination of (a) to (c).

168. The system of any one of claims 157-167, wherein the subject specific data comprises:
(a) a level of one or more analytes in a biological sample obtained from the subject, wherein the one or more analytes comprises (1) a biologic drug, (2) autoantibodies against the biologic drug,
(3) albumin, (4) IL-6, (5) CRP, or (6) any combination of (1) to (5);
(b) a weight of the subject;
(c) BMI of the subject; or
(d) any combination of (a) to (c).

169. The system of claim 168, wherein the dose of the biologic drug at the inter-dose interval to achieve the threshold biologic drug concentration value in the subject is estimated by estimating a clearance rate of the biologic drug in the subject based, at least in part, on the weight of the subject and the level of albumin in the biological sample obtained from the subject in the newly received data from the subject.

170. The system of claim 169, wherein estimating the dose of the biologic drug at the inter-dose interval to achieve the threshold biologic drug concentration value in the subject further comprises determining whether the subject has a poor prognostic factor of pharmacokinetic origin (PPFPK), wherein the PPFPK is determined based, at least in part, on the level of the biologic drug in (a)(1) and the clearance rate.

171. The system of any one of claims 168-170, wherein the level of one or more analytes in a biological sample obtained from the subject is measured using a mobility shift assay or a solid-phase immunoassay.

172. The system of claim 171, wherein the solid-phase immunoassay comprises an enzyme-linked immunoassay (ELISA).

173. The system of any one of claims 168-172, wherein the biological sample comprises a serum sample.

174. The system of any one of claims 168-173, wherein the biological sample is obtained from the subject up to 20 days following a last administration of the biologic drug to the subject.

175. The system of any one of claims 157-174, wherein the model comprises a trained model.

176. The system of claim 175, wherein the model comprises a Bayesian assimilation.

177. The system of claim 175, wherein the model comprises a non-linear mixed effects model (NLME).

178. The system of claim 175, wherein the model comprises a Markov Chain Monte Carlo (MCMC) simulation.

179. The system of any one of claims 157-178, wherein estimating the dose of the biologic drug at the inter-dose interval is performed with greater than a 50% confidence.

180. The system of any one of claims 157-178, wherein estimating the dose of the biologic drug at the inter-dose interval is performed with between about 50% and 90% confidence.

181. The system of any one of claims 157-178, wherein estimating the dose of the biologic drug at the inter-dose interval is performed with greater than or equal to about a 90% confidence.

182. The system of any one of claims 157-181, wherein the biologic drug comprises an antibody or antigen-binding fragment thereof.

183. The system of claim 182, wherein the antibody comprises a monoclonal antibody.

184. The system of any one of claims 157-181, wherein the biologic drug comprises adalimumab (ADA).

185. The system of claim 184, wherein the dose of the ADA is 40 mg and the inter-dose interval is every two weeks.

186. The system of claim 184, wherein the dose of the ADA is less than or equal to about 80 mg and the inter-dose interval is greater than or equal to about every week.

187. The system of any one of claims 157-181, wherein the biologic drug comprises infliximab (IFX).

188. The system of claim 187, wherein the dose of the IFX is 5 mg/kg and the inter-dose interval is every eight weeks.

189. The system of claim 187, wherein the dose of the IFX is less than or equal to about 15 mg/kg and the inter-dose interval is greater than or equal to about every four weeks.

190. The system of any one of claims 157-181, wherein the biologic drug comprises tocilizumab (TCZ).

191. The system of claim 190, wherein the dose of the TCZ is 162 mg and the inter-dose interval is every two weeks.

192. The system of claim 190, wherein the dose of the TCZ is less than or equal to about 162 mg and the inter-dose interval is greater than or equal to about twice every week.

193. The system of any one of claims 157-192, wherein the threshold biologic drug concentration value comprises between about 1 mg/L and 10 mg/L.

194. The system of claim 193, wherein the threshold biologic drug concentration value comprises about 5 to 10 mg/L when the biologic drug is ADA.

195. The system of claim 193, wherein the threshold biologic drug concentration value comprises about 5 to 10 mg/L when the biologic drug is IFX.

196. The system of claim 193, wherein the threshold biologic drug concentration value comprises about 1 to 7.5 mg/L when the biologic drug is TCZ.

197. The system of any one of claims 157-196, further comprising a software module configured to provide a treatment recommendation based on the dose and inter-dose interval of the biologic drug estimated to achieve the threshold biologic drug concentration value in the subject.

198. The system of claim 197, wherein the treatment recommendation comprises:
(a) continuing a current treatment regimen comprising a current dose of the biologic drug at a current inter-dose interval; or
(b) administering a dose of the biologic drug that is lower than the current dose to the subject at an inter-dose interval that is shorter than the current inter-dose interval; provided, in either (a) or (b), that the current dose of the biologic at the inter-dose interval is estimated to achieve the pre-specified threshold concentration of the biologic drug in the subject with a probability of greater than 50%.

199. The system of claim 198, wherein the treatment recommendation comprises administering to the subject a dose of the biologic drug that is higher than a current dose of the biologic that the subject is currently receiving in an inter-dose interval that is shorter than the current inter-dose interval, provided that the current dose of the biologic at the inter-dose interval is estimated to achieve the pre-specified threshold concentration of the biologic drug in the subject with a probability of lower than or equal to about 50%.

200. The system of claim 198, wherein the treatment recommendation comprises discontinuing treatment of the immune-mediated inflammatory disease with the biologic drug if the dose of the biologic drug is above a maximum dose amount.

201. The system of claim 200, wherein the maximum dose amount is 80 mg when the biologic drug comprises ADA.

202. The system of claim 200, wherein the maximum dose amount is 15 mg/kg when the biologic drug comprises IFX.

203. The system of claim 200, wherein the maximum dose amount is 162 mg when the biologic drug comprises TCZ.

204. The system of claim 200, wherein the recommendation further comprises a treatment regimen comprising a small molecule inhibitor of a Janus Kinase (JAK) or a sphingosine 1-phosphate (S1P) receptor modulator.

205. The system of claim 204, wherein:
(a) the small molecule inhibitor of JAK comprises baricitinib, tofacitinib, or upadacitinib, or any combination thereof; and
(b) wherein the S1P receptor modulator comprises fingolimod, siponimod, ozanimod, or ponesimod, or any combination thereof.

206. The system of any one of claims 157-205, wherein the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

207. The system of claim 206, wherein the IBD comprises Crohn's disease (CD).

208. The system of claim 206, wherein the IBD comprises ulcerative colitis (UC).

209. The system of any one of claims 157-208, wherein the subject has received a treatment comprising a current dose of the biologic drug administered to the subject at a current inter-dose interval for at least 14 contiguous weeks.

210. The system of any one of claims 157-208, wherein the subject has received a treatment regimen comprising a current dose of the biologic drug administered to the subject at a current inter-dose interval at least once.

211. A platform comprising:
the computer-implemented system of any one of claim 157 to 210; and
an output device operatively connected to the computing device, wherein the output device is configured to produce a test report comprising the dose of the biologic drug and the inter-dose interval estimated to achieve the threshold biologic drug concentration value in the subject.

212. Non-transitory computer-readable storage media encoded with a computer program including instructions executable by one or more processors for achieving a threshold biologic drug concentration value in a subject, wherein the instructions comprise:
(a) initializing a model of a biologic drug concentration profile for a biologic drug, wherein the model comprises data received from a reference population that were or currently are being treated with the biologic drug for treatment of an immune-mediated inflammatory disease;
(b) generating subject specific parameters relating to pharmacokinetic performance of the biologic drug in the subject;
(c) simulating the biologic drug concentration profile for the subject based on the subject specific parameters and the data from the reference population;
(d) updating the model based on newly received data from the subject in (b) and newly received data from the reference population in (a); and
(e) estimating a dose of the biologic drug at an inter-dose interval to achieve the threshold biologic drug concentration value in the subject with the model updated in (d), wherein the threshold biologic drug concentration is sufficient to treat the immune-mediated inflammatory disease in the subject.

213. The media of claim 212, wherein generating the subject specific parameters comprises compiling information about the subject.

214. The media of claim 213, wherein the information about the subject comprises a severity of the immune-mediated inflammatory disease or a symptom thereof.

215. The media of claim 214, wherein the data is received from the subject via a mobile application on a personal electronic device of the subject.

216. The media of claim 215, wherein the severity of the immune-mediated inflammatory disease comprises a disease remission, a disease recurrence, a disease type, or any combination thereof.

217. The media of claim 215 or 216, wherein the severity of the symptom of the immune-mediated inflammatory disease comprises a frequency of the symptom, a type of the symptom, or a combination thereof.

218. The media of any one of claims 215-217, wherein the severity of the immune-mediated inflammatory disease or symptom thereof is based on a clinical disease activity index (CDAI) score.

219. The media of any one of claims 213-218, wherein the information about the subject comprises a weight or body mass index (BMI) of the subject.

220. The media of any one of claims 212-219, wherein the data is contained in one or more electronic medical records (EMRs).

221. The media of any one of claims 212-220, wherein the subject specific parameters comprise two or more of:
(a) a clearance (C);
(b) a volume of distribution of a central compartment (Vc);
(c) intercompartmental clearance;
(d) a volume of a peripheral compartment (Vp);
(e) absorption rate constant;
(f) maximum velocity at high biologic drug concentrations (Vmax);
(g) affinity of the biologic drug to a substrate;
(h) proportional error;
(i) body weight; or
(j) log transformed covariates on the subject specific parameters in one or more of (a) to (g) as determined using non-linear mixed effect modeling; or
(k) any combination thereof.

222. The media of any one of claims 212-221, wherein the data received from the reference population comprises:
(a) a level of one or more analytes comprising (1) a biologic drug, (2) autoantibodies against the biologic drug, (3) albumin, (4) interleukin 6 (IL-6), (5) C-Reactive Protein (CRP), or (6) any combination of (1) to (5);
(b) a weight of individuals in the reference population;
(c) a body mass index (BMI) of the individuals in the reference population; or
(d) any combination of (a) to (c).

223. The media of any one of claims 212-221, wherein the newly received data from the subject comprises:
(a) a level of one or more analytes in a biological sample obtained from the subject, wherein the one or more analytes comprises (1) a biologic drug, (2) autoantibodies against the biologic drug, (3) albumin, (4) IL-6, (5) CRP, or (6) any combination of (1) to (5);
(b) a weight of the subject;
(c) BMI of the subject; or
(d) any combination of (a) to (c).

224. The media of any one of claims 212-221, wherein estimating the dose of the biologic drug at the inter-dose interval to achieve the threshold biologic drug concentration value in the subject comprises estimating a clearance rate of the biologic drug in the subject based, at least in part, on the weight of the subject and the level of albumin in the biological sample obtained from the subject in the newly received data from the subject.

225. The media of claim 224, wherein estimating the dose of the biologic drug at the inter-dose interval to achieve the threshold biologic drug concentration value in the subject further comprises determining whether the subject has a poor prognostic factor of pharmacokinetic origin (PPFPK), wherein the PPFPK is determined based, at least in part, on the level of the biologic drug in (a)(1) and the clearance rate.

226. The media of any one of claims 212-225, wherein the level of one or more analytes in a biological sample obtained from the subject is measured using a mobility shift assay or a solid-phase immunoassay.

227. The media of claim 226, wherein the solid-phase immunoassay comprises an enzyme-linked immunoassay (ELISA).

228. The media of any one of claims 212-227, wherein the biological sample comprises a serum sample.

229. The media of any one of claims 212-228, wherein the biological sample is obtained from the subject up to 20 days following a last administration of the biologic drug to the subject.

230. The media of any one of claims 212-229, wherein the model comprises a trained model.

231. The media of claim 230, wherein the model comprises a Bayesian assimilation.

232. The media of claim 230, wherein the model comprises anon-linear mixed effects model (NLME).

233. The media of claim 230, wherein the model comprises a Markov Chain Monte Carlo (MCMC) simulation.

234. The media of any one of claims 212-233, wherein the dose of the biologic drug at the inter-dose interval is estimated with a probability greater than a 50%.

235. The media of any one of claims 212-233, wherein the dose of the biologic drug at the inter-dose interval is estimated with a probability between about 50% and 90%.

236. The media of any one of claims 212-233, wherein the dose of the biologic drug at the inter-dose interval is estimated with a probability of greater than or equal to about a 90%.

237. The media of any one of claims 212-236, wherein the biologic drug comprises an antibody or antigen-binding fragment thereof.

238. The media of claim 237, wherein the antibody comprises a monoclonal antibody.

239. The media of any one of claims 212-236, wherein the biologic drug comprises adalimumab (ADA).

240. The media of claim 239, wherein the dose of the ADA is 40 mg and the inter-dose interval is every two weeks.

241. The media of claim 239, wherein the dose of the ADA is less than or equal to about 80 mg and the inter-dose interval is greater than or equal to about every week.

242. The media of any one of claims 212-236, wherein the biologic drug comprises infliximab (IFX).

243. The media of claim 242, wherein the dose of the IFX is 5 mg/kg and the inter-dose interval is every eight weeks.

244. The media of claim 242, wherein the dose of the IFX is less than or equal to about 15 mg/kg and the inter-dose interval is greater than or equal to about four weeks.

245. The media of any one of claims 212-236, wherein the biologic drug comprises tocilizumab (TCZ).

246. The media of claim 245, wherein the dose of the TCZ is 162 mg and the inter-dose interval is every two weeks.

247. The media of claim 245, wherein the dose of the TCZ is less than or equal to about 162 mg and the inter-dose interval is greater than or equal to about twice every week.

248. The media of any one of claims 212-247, wherein the threshold biologic drug concentration value comprises between about 1 mg/L and 10 mg/L.

249. The media of claim 248, wherein the threshold biologic drug concentration value comprises about 5 to 10 mg/L when the biologic drug is ADA.

250. The media of claim 248, wherein the threshold biologic drug concentration value comprises about 5 to 10 mg/L when the biologic drug is IFX.

251. The media of claim 248, wherein the threshold biologic drug concentration value comprises about 1 to 7.5 mg/L when the biologic drug is TCZ.

252. The media of any one of claim 212-251, wherein the instructions further comprises providing a recommendation to discontinue treatment of the immune-mediated inflammatory disease with the biologic drug if the dose of the biologic drug is above a maximum dose amount.

253. The media of claim 252, wherein the maximum dose amount is 80 mg when the biologic drug comprises ADA.

254. The media of claim 252, wherein the maximum dose amount is 15 mg/kg when the biologic drug comprises IFX.

255. The media of claim 252, wherein the maximum dose amount is 162 mg when the biologic drug comprises TCZ.

256. The media of claim 252, wherein the recommendation further comprises a treatment regimen comprising a small molecule inhibitor of a Janus Kinase (JAK) or a sphingosine 1-phosphate (S1P) receptor modulator.

257. The media of claim 256, wherein:
(a) the small molecule inhibitor of JAK comprises baricitinib, tofacitinib, or upadacitinib, or any combination thereof; and
(b) wherein the S1P receptor modulator comprises fingolimod, siponimod, ozanimod, or ponesimod, or any combination thereof.

258. The media of any one of claims 212-257, wherein the subject has or is suspected of having an immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

259. The media of claim 258, wherein the IBD comprises Crohn's disease (CD).

260. The media of claim 258, wherein the IBD comprises ulcerative colitis (UC).

261. The media of any one of claims 212-260, wherein the subject has received a treatment comprising a current dose of the biologic drug administered to the subject at a current inter-dose interval for at least 14 contiguous weeks.

262. The media of any one of claims 212-260, wherein the subject has received a treatment regimen comprising a current dose of the biologic drug administered to the subject at a current inter-dose interval at least once.

263. A method of treating an immune-mediated inflammatory disease of a subject, the method comprising:
(a) performing or having performed an immunoassay on a biological sample obtained from the subject to determine a level of albumin and a level of a biologic drug that are predictive of clinical remission of the immune-mediated inflammatory disease of the subject, wherein the subject is currently receiving the biologic drug for treatment of the immune-mediated inflammatory disease;
(b) estimating a clearance rate of the biologic drug for the subject based, at least in part, on the level of albumin determined in (a) and a weight of the subject; and
(c) if the level of the biologic drug is above a cutoff level in milligrams/L (mg/L) and the clearance rate is estimated to be below a threshold level of liters (L)/day, then administering a lower dose of the biologic drug to the subject or discontinuing the treatment of the immune-mediated inflammatory disease with the biologic drug; or
(d) if the level of the biologic drug is below the cutoff level and the clearance rate is estimated to be above the threshold level, then administering the biologic drug to the subject in the same or higher dose than a dose of the biologic drug the subject is currently receiving, or administering a different drug to the subject, wherein the threshold level and the cutoff level are derived from an optical Youden index.

264. The method of claim 263, wherein the level of the biologic drug is between about 3 mg/L to about 10 mg/L.

265. The method of claim 263, wherein the clearance rate is estimated to be between about 0.20 L/d to about 0.4 L/d.

266. The method of any one of claims 263-264, wherein the threshold level is about 0.25 L/day.

267. The method of any one of claims 263-265, wherein the cutoff level is about 10 mg/L.

268. The method of any one of claims 263-267, wherein the biologic drug comprises an antibody or an antigen-binding fragment.

269. The method of claim 266, wherein the antibody comprises a monoclonal antibody.

270. The method of any one of claims 263-269, wherein the biologic drug comprises IFX.

271. The method of any one of claims 263-269, wherein the biologic drug comprises TCZ.

272. The method of any one of claims 263-269, wherein the biologic drug comprises ADA.

273. The method of any one of claims 263-272, wherein the different drug comprises a small molecule inhibitor of Janus Kinase (JAK) or a sphingosine 1-phosphate (S1P) receptor modulator.

274. The method of claim 273, wherein:
(a) the small molecule inhibitor of JAK comprises baricitinib, tofacitinib, or upadacitinib, or any combination thereof; and
(b) wherein the SIP receptor modulator comprises fingolimod, siponimod, ozanimod, or ponesimod, or any combination thereof.

275. The method of any one of claims 263-274, wherein the immunoassay comprises an enzyme-linked immunoassay (ELISA) or a mobility shift assay.

276. The method of any one of claims 263-275, wherein the immune-mediated inflammatory disease comprises an inflammatory bowel disease (IBD), rheumatoid arthritis (RA), cytokine release syndrome, multiple sclerosis (MS), ankylosing spondylitis (AS), lupus, plaque psoriasis, atopic dermatitis, gout, migraine, cancer, or a neoplasm.

277. The method of claim 276, wherein the IBD comprises Crohn's disease (CD).

278. The method of claim 276, wherein the IBD comprises ulcerative colitis (UC).

279. The method of any one of claims 263-278, wherein the estimating the clearance rate of the biologic drug of the subject comprises:
(a) inputting the level of albumin in the biological sample obtained from the subject and the weight of the subject into a model, wherein the model has been trained using pharmacokinetic data from a reference population; and
(b) outputting an estimated clearance rate of the biologic drug for the subject.

280. The method of claim 279, wherein the model comprises a Bayesian assimilation.

281. The method of claim 279, wherein the model comprises a non-linear mixed effects model (NLME).

282. The method of claim 279, wherein the model comprises a Markov Chain Monte Carlo (MCMC) simulation.

283. The method of any one of claims 279-282, wherein the reference population is comprised of reference subjects with the immune-mediated inflammatory disease who have received treatment with the biologic drug for the immune-mediated inflammatory disease.
